(19) United States
(12) Patent Application Publication
(10) Pub. No.: US 2013/0058993 A1
(43) Pub. Date: Mar. 7, 2013

(54) METHODS AND COMPOSITIONS FOR ENHANCING WOUND HEALING USING CAR PEPTIDES

(71) Applicants: Erkki Ruoslahti, Buellton, CA (US); Tero Jarvinen, Tampere (FI)

(72) Inventors: Erkki Ruoslahti, Buellton, CA (US); Tero Jarvinen, Tampere (FI)

(73) Assignee: Sanford-Burnham Medical Research Institute

(21) Appl. No.: 13/406,699

(22) Filed: Feb. 28, 2012

Related U.S. Application Data
(63) Continuation-in-part of application No. 13/224,193, filed on Sep. 1, 2011.
(60) Provisional application No. 61/597,076, filed on Feb. 9, 2012.

(51) Int. Cl. A61K 38/08; A61K 35/76; C07K 7/06; A61K 38/02; A61P 9/12; A61P 7/02; A61P 29/00; A61P 9/00; A61P 19/02; A61P 9/08; A61P 21/02; A61P 11/00; A61P 37/00; A61K 9/127; B82Y 5/00

(52) U.S. Cl. 424/450; 514/21.6; 514/21.7; 424/93.6; 530/329; 530/328; 514/1.1; 514/15.7; 514/13.7; 977/773; 977/906

(57) ABSTRACT

Disclosed are compositions and methods useful for treating wounded, injured, and inflamed tissue. The compositions and methods are based on peptide sequences, such as CAR peptides and truncated CAR peptides, that are selectively targeted to wounded tissue and are internalized by a cell, penetrate tissue, or both. The disclosed peptides promote and enhance wound healing.

### CROSS REFERENCE TO RELATED APPLICATIONS

This application is a continuation-in-part of U.S. application Ser. No. 13/224,193, filed Sep. 1, 2011. This application also claims benefit of U.S. Provisional Application No. 61/597,076, filed Feb. 9, 2012. U.S. application Ser. No. 13/224,193, filed Sep. 1, 2011, and U.S. Provisional Application No. 61/597,076, filed Feb. 9, 2012, are hereby incorporated herein by reference in their entirety.

### STATEMENT REGARDING FEDERALLY SPONSORED RESEARCH

This invention was made with government support under grant 1 R41 HL088771 from the National Heart Lung Blood Institute (NHLBI) of the National Institutes of Health (NIH). The government has certain rights in the invention.

### REFERENCE TO SEQUENCE LISTING

The Sequence Listing submitted Feb. 28, 2012, as a text file named “SBMRI_{—}59_{—}8402_AMD_AFD_Sequence_Lisiting_text_file.txt,” created on Feb. 28, 2012, and having a size of 37,924 bytes is hereby incorporated by reference pursuant to 37 C.F.R. §1.52(e)(5).

### FIELD OF THE INVENTION

The present invention relates generally to the field of molecular medicine, wound healing, and more specifically, to cell and tissue-targeting peptides.

### BACKGROUND OF THE INVENTION

Peptides that are internalized into cells are commonly referred to as cell-penetrating peptides. There are two main classes of such peptides: hydrophobic and cationic (Zorko and Langel, 2005). The cationic peptides, which are commonly used to introduce nucleic acids, proteins into cells, include the prototypic cell-penetrating peptides (CPP), Tat, and penetratin (Derossi et al., 1998; Meade and Dowdy, 2007). A herpes virus protein, VP22, is capable of both entering and exiting cells and carrying a payload with it (Elliott and O'Hare, 1997; Brewis et al., 2003). A major limitation of these peptides as delivery vehicles is that they are not selective; they enter into all cells.

Tissue penetration is a serious limitation in the delivery of compositions to cells. It is important to find new ways of improving the passage of diverse compositions into the extravascular space. A number of proteins are known to translocate through the endothelium of blood vessels, including the blood-brain barrier. A prime example is transferrin, which is carried across the blood-brain barrier by the transferrin receptor. This system has been used to bring other payloads into the brain (Li et al., 2002; Fenart and Cecchelli, 2003). Peptide signals for endothelial transcytosis that can mediate translocation of compositions from the circulation into tissues is useful.

Tissue regeneration, inflammation and tumors induce the growth of new blood vessels from pre-existing ones. This process, angiogenesis, is a vital requirement for wound healing as the formation of new blood vessels allows a variety of mediators, nutrients, and oxygen to reach the healing tissue (Martin 1997, Singer & Clark 1999, Falanga 2006, Folkman 2006). Newly formed blood vessels differ in structure from pre-existing vasculature. Such differences have been extensively characterized by comparing tumor vasculature to normal vessels (Ruoslahti, 2002). Angiogenic vessels in non-malignant tissues and in pre-malignant lesions share markers with tumor vessels (Gerlag et al, 2001), but distinct markers also exist (Hoffman et al., 2003; Joyce et al., 2003).

Regarding tissue injuries, substantive basic science and clinical research have been conducted to evaluate the mechanisms of wound healing, the efficacy of various modalities for treatment of wounds, and the best methods for diagnosing wound infection. Tissue injuries caused by trauma, medical procedures, and inflammation are a major medical problem. Systemic medication is available for most major medical conditions, but therapeutic options in promoting tissue regeneration are largely limited to local intervention. As deep injuries and multiple sites of injury often limit the usefulness of local treatment, systemic approaches to tissue regeneration are valuable.

A major problem limiting tissue regeneration is scar formation. The response to tissue injury in adult mammals seems to be mainly focused on quick sealing on the injury. Fibroblast (astrocyte, smooth muscle cell) proliferation and enhanced extracellular matrix production are the main element of the scar formation, and the scar prevents tissue regeneration. In contrast, fetal tissues heal by a process that restores the original tissue architecture with no scarring. Transforming growth factor-beta (TGF-beta) is a major factor responsible for impaired tissue regeneration, scar formation and fibrosis (Werner and Grose 2002; Brunner and Blakytny 2004; Leask and Abraham 2004).

One manner by which therapeutic specificity may be increased is by targeting diseases at the cellular level. More specifically, therapeutics may be enhanced by interacting directly with those components at the level of the cell surface or membrane. These components include, among others, laminin, collagen, fibronectin and other proteoglycans. Proteoglycans are proteins classified by a posttranslational attachment of polysaccharide glycosaminoglycan (GAG) moieties each comprised of repeating disaccharide units. One monosaccharide of the disaccharide repeat is an amino sugar with D-glucosamine or galactosamine, and the other unit is typically, but not always, a uronic acid residue of either D-glucuronic acid or iduronic acid. Both units are variably N- and O-sulfated, which adds to the heterogeneity of these complex macromolecules. They can be found associated with both the extracellular matrix and plasma membranes. The most common GAG structures are dermatan sulfate (DS), chondroitin sulfate (CS), heparan sulfate (HS), keratan sulfate (KS), hyaluronic acid (HA), and heparin; representative structures for each disaccharide are shown below.

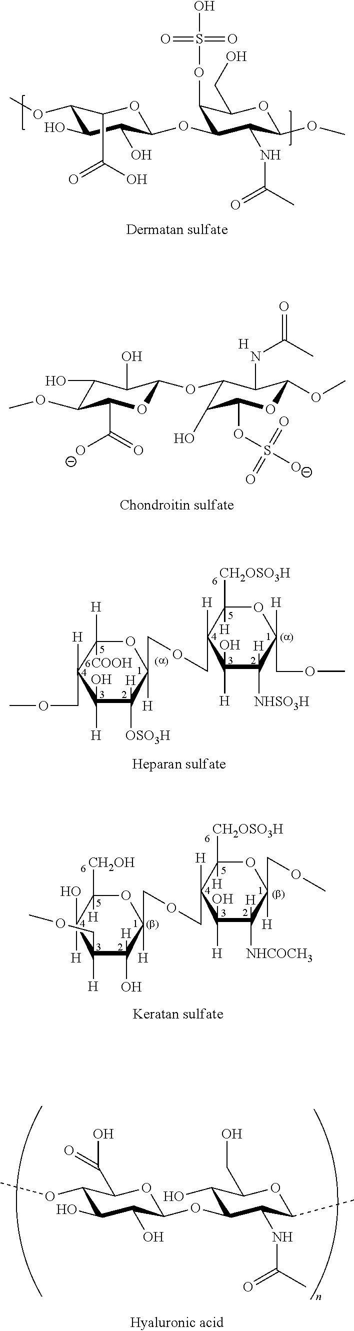

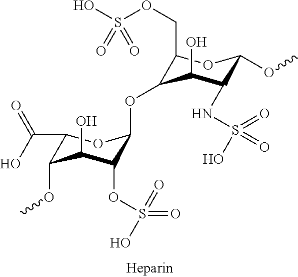

These unbranched sulfated GAGs are defined by the repeating disaccharide units that comprise their chains, by their specific sites of sulfation, and by their susceptibility to bacterial enzymes known to cleave distinct GAG linkages. All have various degrees of sulfation which result in a high density of negative charge. Proteoglycans can be modified by more than one type of GAG and have a diversity of functions, including roles in cellular adhesion, differentiation, and growth. In addition, cell surface proteoglycans are known to act as cellular receptors for some bacteria and several animal viruses, including; foot-and-mouth disease type O virus, HSV types 1 and 2 and dengue virus. Accordingly, it would be advantageous from a therapeutic perspective to design agents which may be used at the cell surface level.

A major function of cell surface proteoglycans is in cell adhesion and migration, dynamic processes that are mediated through interactions between the proteoglycan GAG chains and extracellular matrix (ECM) components, such as laminin, collagen, and fibronectin. Proteoglycans also occur as integral components of basement membranes in most mammalian tissues. Interactions of these macromolecules with other ECM constituents contribute to the general architecture and permeability properties of the basement membrane, and thus these GAGs play a structural role. Proteoglycans and GAGs play a critical role in the pathophysiology of basement membrane-related diseases, including diabetes, atherosclerosis, and metastasis. In addition, cell-specific growth factors and enzymes are immobilized in the ECM and at the cell surface are bound to GAGs. As such, GAGs localize proteins and enzymes at their site of action to facilitate their physiological functions and in some cases prevent their proteolytic degradation. Proteoglycans and GAGs have been shown to regulate protein secretion and gene expression in certain tissues by mechanisms involving both membrane and nuclear events, including the binding of GAGs to transcription factors (Jackson, R. L. 1991). Limited information is available on the factors that regulate the expression of proteoglycans and their associated GAGs. There is a need in the art to develop cell-penetrating agents which bind to cell surface proteoglycans in order to have disease-specific efficacy.

HS2ST1 (heparan sulfate 2-O-sulfotransferase 1) catalyzes the transfer of sulfate to the C2-position of selected hexuronic acid residues within the maturing heparan sulfate. EXT1 (exostosin 1) is an endoplasmic reticulum-resident type II transmembrane glycosyltransferase involved in the chain elongation step of heparan sulfate biosynthesis. GLT8D2 (glycosyltransferase 8 domain containing 2) is an enzyme involved in HSPG biosynthesis. NDST1 (Heparan sulfate N-deacetylase/N-sulfotransferase) is a HSPG biosynthetic enzyme. OGT (O-linked N-acetylglucosamine (O-GlcNAc) transferase) catalyzes the addition of a single N-acetylglucosamine in β-glycosidic linkage to serine or threonine residues of intracellular proteins including HSPGs.

US Patent Application Publication No. 20090036349 discloses a novel composition that selectively binds to regenerating tissue, wound sites and tumors in animals. In vivo screening of phage-displayed peptide libraries was used to probe vascular specialization. This screening method resulted in the identification of several peptides that selectively target phage to skin and tendon wounds. One peptide in particular was identified and contains the following sequence: CARSKNKDC (CAR) (SEQ ID NO:10). CAR displays homology to heparin-binding sites in various proteins, and binds to cell surface heparan sulfate and heparin. More specifically, CAR binds to glycosaminoglycan moieties in cell surface heparan sulfate proteoglycans (HSPGs) (Jarvinen and Ruoslahti 2007), and other cell-penetrating peptides have also mediated their entry into cells through binding to HSPGs (Poon and Gariépy 2007). HSPGs fine-tune mammalian physiology and orchestrate metabolism, transport, information transfer, support and regulation at the systemic level, as well as the cellular level (Bishop, Schuksz and Esko 2007). Overexpression of HSPG biosynthetic enzymes result in distinct heparan sulfate sulfation patterns (Pikas, Erikson and Kjellen 2000). The overexpression of HSPG biosynthetic enzymes have not been previously detected in a disease in which the co-administration of a cell penetrating peptide along with a bioactive agent which results in the disease-selective action of the co-administration of the peptide/agent combination.

Thus, there is a need for new therapeutic strategies for selectively targeting various types of cells, and for enhancing wound healing. The present invention satisfies these needs by providing peptides, compositions, and methods that can be selectively targeted to wounded tissue and which promote and enhance wound healing. Related advantages also are provided.

### BRIEF SUMMARY OF THE INVENTION

Disclosed are peptides that target regenerating tissue, wounded tissue, pulmonary tissue, fibrotic tissue, and related tissue, that enhance wound healing, that are readily internalized into adjacent cells, and that extensively penetrate and invade wounded tissue. The disclosed peptides and compositions can also mediate targeting, internalization, and tissue penetration of compounds and compositions coupled to, associated with, conjugated to, or even co-administered with the peptide. Examples of the disclosed peptides include peptides having the sequence CARSKNKDC (CAR; SEQ ID NO:10) or CARSKNK (truncated CAR; tCAR; SEQ ID NO:4) or peptides consisting of CARSKNKDC (SEQ ID NO:10) or CARSKNK (SEQ ID NO:4). For example, disclosed are peptides where the C-terminal end of the peptide consists of the amino acid sequence CARSKNKDC (SEQ ID NO:10) or the amino acid sequence CARSKNK (SEQ ID NO:4), peptides where the N-terminal end of the peptide consists of the amino acid sequence CARSKNKDC (SEQ ID NO:10) or CARSKNK (SEQ ID NO:4), and peptides where neither the N-terminal end of the peptide nor the C-terminal end of the peptide consists of the amino acid sequence CARSKNKDC (SEQ ID NO:10) or CARSKNK (SEQ ID NO:4). The disclosed peptides can be used in and with a variety of compositions and methods to, for example, enhance wound healing.

Disclosed are compositions and methods that can be used to enhance wound healing. For example, disclosed are compositions comprising a wound therapeutic, where the only wound therapeutic in the composition is an isolated CAR peptide and/or an isolated truncated CAR peptide. Also disclosed are compositions comprising an isolated peptide, wherein the peptide comprises the amino acid sequence of a CAR peptide and/or a truncated CAR peptide, where the amount of peptide in the composition is a wound healing effective amount. Also disclosed are compositions consisting essentially of an isolated peptide, wherein the peptide comprises the amino acid sequence of a CAR peptide and/or a truncated CAR peptide. Also disclosed are compositions comprising an isolated peptide, wherein the C-terminal end of the peptide consists of the amino acid sequence of a CAR peptide and/or a truncated CAR peptide, where the amount of peptide in the composition is a wound healing effective amount. Also disclosed are compositions consisting essentially of an isolated peptide, wherein the C-terminal end of the peptide consists of the amino acid sequence of a CAR peptide and/or a truncated CAR peptide.

Also disclosed are compositions comprising a wound therapeutic, where the only wound therapeutic in the composition is an isolated peptide, wherein the peptide comprises the amino acid sequence CARSKNKDC (SEQ ID NO:10) or the amino acid sequence CARSKNK (SEQ ID NO:4). Also disclosed are compositions comprising an isolated peptide, wherein the peptide comprises the amino acid sequence CARSKNKDC (SEQ ID NO:10) or the amino acid sequence CARSKNK (SEQ ID NO:4), where the amount of peptide in the composition is a wound healing effective amount. Also disclosed are compositions consisting essentially of an isolated peptide, wherein the peptide comprises the amino acid sequence CARSKNKDC (SEQ ID NO:10) or the amino acid sequence CARSKNK (SEQ ID NO:4). Also disclosed are compositions comprising a wound therapeutic, where the only wound therapeutic in the composition is an isolated peptide, wherein the C-terminal end of the peptide consists of the amino acid sequence CARSKNKDC (SEQ ID NO:10) or the amino acid sequence CARSKNK (SEQ ID NO:4). Also disclosed are compositions comprising an isolated peptide, wherein the C-terminal end of the peptide consists of the amino acid sequence CARSKNKDC (SEQ ID NO:10) or the amino acid sequence CARSKNK (SEQ ID NO:4), where the amount of peptide in the composition is a wound healing effective amount. Also disclosed are compositions consisting essentially of an isolated peptide, wherein the C-terminal end of the peptide consists of the amino acid sequence CARSKNKDC (SEQ ID NO:10) or the amino acid sequence CARSKNK (SEQ ID NO:4). Also disclosed are methods of enhancing wound healing comprising exposing wounded tissue to one or more of the disclosed compositions, thereby enhancing wound healing.

In some forms, the peptide can be a modified peptide. In some forms, the peptide can be a methylated peptide. In some forms, the methylated peptide can comprise a methylated amino acid segment. In some forms, the peptide can be N- or C-methylated in at least one position. In some forms, the peptide can be an activatable peptide. In some forms, the amino acid sequence CARSKNKDC (SEQ ID NO:10) or CARSKNK (SEQ ID NO:4) can be blocked by a blocking group. The blocking group can be coupled to the terminal carboxy group, the terminal amino group, the C-terminal amino acid, the N-terminal amino acid, an amino acid in the CAR or tCAR amino acid sequence, or elsewhere in the peptide. In some forms, the amino acid sequence at the C-terminal end of the peptide can be blocked by a blocking group coupled to the terminal carboxy group. In some forms, the blocking group can comprise an amino acid or an amino acid sequence. In some forms, the amino acid sequence CARSKNKDC (SEQ ID NO:10) or the amino acid sequence CARSKNK (SEQ ID NO:4) can be at the C-terminal end of the peptide. In some forms, the amino acid sequence CARSKNKDC (SEQ ID NO:10) or the amino acid sequence CARSKNK (SEQ ID NO:4) can be at the N-terminal end of the peptide. In some forms, the amino acid sequence CARSKNKDC (SEQ ID NO:10) or the amino acid sequence CARSKNK (SEQ ID NO:4) is not at the N- or C-terminal end of the peptide.

In some forms, the peptide can selectively home to regenerating tissue, a site of injury, a surgical site, a site of inflammation, a site of arthritis, pulmonary arterial hypertension lung vasculature, pulmonary arterial hypertension lesions, remodeled pulmonary arteries, or interstitial space of lungs. In some forms, the composition can selectively home to regenerating tissue, a site of injury, a surgical site, a site of inflammation, a site of arthritis, pulmonary arterial hypertension lung vasculature, pulmonary arterial hypertension lesions, remodeled pulmonary arteries, or interstitial space of lungs.

In some forms, the composition can further comprise a carrier, vehicle, or both. In some forms, the composition can further comprise a carrier, a vehicle, a virus, a phage, a viral particle, a phage particle, a viral capsid, a phage capsid, a virus-like particle, a liposome, a micelle, a bead, a nanoparticle, a microparticle, or a combination. In some forms, the composition can further comprise a plurality of copies of the peptide.

In some forms, the peptide can be comprised in a conjugate. In some forms, the conjugate can comprise one or more homing molecules. In some forms, the composition can further comprise one or more homing molecules. In some forms, the homing molecules can selectively home to lung vasculature, regenerating tissue, wounded tissue, pulmonary arterial hypertension lung vasculature, pulmonary arterial hypertension lesions, remodeled pulmonary arteries, interstitial space of lungs, a site of injury, a surgical site, a site of inflammation, or a site of arthritis. In some forms, the homing molecules can be conjugated with a surface molecule. In some forms, one or more of the conjugated homing molecules can be indirectly conjugated to the surface molecule via a linker. In some forms, the composition can further comprise a plurality of linkers. In some forms, at least one of the linkers can comprise polyethylene glycol.

In some forms, the composition can bind inside blood vessels of regenerating tissue, blood vessels of wounded tissue, or lung blood vessels. In some forms, the composition can be internalized in cells. In some forms, the composition can penetrate tissue. In some forms, one or more of the homing molecules can be modified homing molecules. In some forms, one or more of the homing molecules can comprise a methylated homing molecule. In some forms, one or more of the methylated homing molecules can comprise a methylated amino acid segment.

In some forms, the composition can further comprise one or more moieties. In some forms, the moieties can be independently selected from the group consisting of a therapeutic agent, a therapeutic protein, a therapeutic compound, a therapeutic composition, a carrier, a vehicle, a virus, a phage, a viral particle, a phage particle, a viral capsid, a phage capsid, a virus-like particle, a liposome, a micelle, a bead, a nanoparticle, a microparticle, or a combination. In some forms, the composition can further comprise one or more accessory molecules.

In some forms, the composition can have a therapeutic effect. In some forms, the therapeutic effect can comprise a reduction in inflammation, an increase in speed of wound healing, reduction in amounts of scar tissue, decrease in pain, decrease in swelling, or decrease in necrosis. In some forms, the therapeutic effect can comprise pulmonary vasodilation, decrease in pulmonary pressure, anti-coagulation, airway smooth muscle relaxation, increase in glutathione (GSH), decrease in inflammatory immune response, inhibition of thromboxane synthesis, or inhibition of leukotriene synthesis.

In some forms, the wounded tissue can be in a subject. In some forms, the wounded tissue can be exposed to the composition by administering the composition to the subject. In some forms, the composition can penetrate tissue. In some forms, the composition can penetrate lung vasculature, regenerating tissue, wounded tissue, pulmonary arterial hypertension lung vasculature, pulmonary arterial hypertension lesions, remodeled pulmonary arteries, interstitial space of lungs, a site of injury, a surgical site, ex vivo tissue, transplant tissue, ex vivo transplant tissue, a site of inflammation, or a site of arthritis. The wounded tissue can be exposed in vivo, ex vivo, or in vitro. For example, tissue to be introduced into a subject can be exposed ex vivo. For example, transplant cells and/or tissue can be exposed in vivo or ex vivo. For example, transplant cells and/or tissue can be exposed prior to, during, and/or following transplantation.

In some forms, the composition further comprises a carrier, a vehicle, a virus, a phage, a viral particle, a phage particle, a viral capsid, a phage capsid, a virus-like particle, a liposome, a micelle, a bead, a nanoparticle, a microparticle, or a combination. In some forms, the wounded tissue can be exposed to a plurality of homing molecules. In some forms, the wounded tissue can be exposed to a plurality of compositions.

In some forms, the subject can have a disease or condition. In some forms, the disease can be pulmonary or fibrotic. In some forms, the disease or condition can be pulmonary arterial hypertension (PAH). In some forms, the disease or condition can be an autoimmune disease. In some forms, the disease or condition can be an inflammatory disease.

In some forms, the subject can have one or more sites to be targeted, where the composition homes to one or more of the sites to be targeted. In some forms, the peptide can selectively home to lung vasculature, regenerating tissue, wounded tissue, pulmonary arterial hypertension lung vasculature, pulmonary arterial hypertension lesions, remodeled pulmonary arteries, interstitial space of lungs, a site of injury, a surgical site, a site of inflammation, or a site of arthritis. In some forms, the composition can selectively home to lung vasculature, regenerating tissue, wounded tissue, pulmonary arterial hypertension lung vasculature, pulmonary arterial hypertension lesions, remodeled pulmonary arteries, interstitial space of lungs, a site of injury, a surgical site, a site of inflammation, or a site of arthritis.

In some forms, the composition can enhance internalization, penetration, or both of one or more blood, plasma, or serum components into or through the wounded tissue. In some forms, the composition is not administered with any other wound therapeutic.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a schematic representation of how serum promotes re-epithelization during wound healing. The three classical and sequential events of wound healing; inflammation, re-epithelialization, and tissue remodeling are schematically depicted. Three major types of cells involved in wound repair, keratinocytes (HKs), dermal fibroblasts (DFs), and human dermal microvascular endothelial cells (HDMECs), are shown. The serum derived TGFα levels are dramatically increased in the wound fluid, following the transition from plasma to serum in the wound bed. After the wound is closed and after transition of serum back to plasma, the levels of TGFα go back to those in unwounded skin (Li et al. 2006).

FIG. 2 shows a graph of wound closure in CAR-treated animals. Mice with full thickness skin wounds received i.v. injections of 75 μg of CAR twice a day from Day 1 after the wounding until sacrifice. The wounds were examined and photographed daily. Wound closure was recorded and expressed as the size of unclosed wound. The image shows accelerated wound closure in wounds treated with CAR compared with the controls groups (PBS and mCAR peptide). Statistical significance was examined using the χ2 test (CAR vs Control/mCAR for all time-points from Day 5 on P<0.0001). n=72 on days 0-5, 48 on days 6-7, and 24 on days 8-10.

FIGS. 3A and 3B show graphs of complete wound closure/re-epithelialization in CAR peptide treated animals. (A) The wounds of animals treated as in FIG. 2 were examined and photographed daily. Wound closure was recorded and expressed as percentage of wounds that had completely closed. (B) Re-epithelialization of the epidermis was quantified on days 5, 7, and 10 by examining two microscopic sections from each wound.

FIGS. 4A, 4B, and 4C show graphs of wound re-epithelialization in peptide-treated mice. (A) The wounds of mice treated as in FIG. 2 were harvested and analyzed microscopically. The gap in the epidermis was quantified on days (A) 5, (B) 7 and (C) 10 by examining two microscopic sections from each wound and expressed as the average of the two values. P<0.001 CAR vs. Control/mCAR at all three time-points, statistical significance was examined using the ANOVA. The results are expressed as mean±SEM, n=24 at all three time-points.

FIG. 5 shows the lung homing efficiency and specificity of targeting peptides. Immunostained lung sections were analyzed for MCT-treated and untreated rats using Aperio's Color Deconvolution to quantify the staining intensity. The peptide-positive area relative to the total lung cell area is shown. Control, CG7C peptide. Three detection thresholds were set to determine weak to strong accumulations of the peptides. Multiple sections were analyzed for each group. Bar indicates mean±S.D.

FIG. 6 is a bar graph of the percent of control without blocking versus different peptides. The graph shows the effect of blocking neuropilin-1 on cell binding and internalization of CAR and tCAR phage. Error bars represent mean±SEM.

FIG. 7 is a bar graph of the fold over control phage shows the internalization of phage displaying CAR or a truncated CAR peptide (tCAR) into cells. Error bars represent mean±SEM.

FIG. 8 shows the effects of acute administration of fasudil with (+CAR) and without co-administration of CAR (1 mg/300 g rat) (−CAR) on right (RVSP) and left ventricle systolic pressure (LVSP). Vasodilator effects were expressed as % reduction of baseline pressure. Values are means of n=1-2 each.

FIGS. 9A-9D shows CAR peptide homing to acute injury. CAR peptide is known to home to angiogenic vasculature at wounds (Jarvinen & Ruoslahti 2007, 2010). CAR homing to wound during the inflammatory phase, i.e. prior to the angiogenesis takes place during tissue repair, was tested. FIGS. 9A-9D: fluorescein-conjugated CAR (FIGS. 9A and 9C) or fluorescein-conjugated control peptides (FIGS. 9B and 9D) (500 μg) were i.v. injected into mice 48 hours after wounding (FIGS. 9A and 9B) and skeletal muscle crush-injuries (FIGS. 9C and 9D). The injured tissue was collected 4 hours later and examined for the presence of the peptides. The nuclei were stained with DAPI. Magnification, FIGS. 9A and 9B×100, FIGS. 9C and 9D×200.

### DETAILED DESCRIPTION OF THE INVENTION

The disclosed method and compositions can be understood more readily by reference to the following detailed description of particular embodiments and the Examples included therein and to the Figures and their previous and following description.

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Every peptide, composition, conjugate, compound, etc. described herein is intended to be and should be considered to be specifically disclosed herein. Further, every subgroup of such peptides, compositions, conjugates, compounds, etc. that can be identified within the disclosure is intended to be and should be considered to be specifically disclosed herein. As a result, it is specifically contemplated that any composition, or subgroup of compositions can be either specifically included for or excluded from use or included in or excluded from a list of compositions. For example, as one option, a group of wound healing compositions is contemplated where each composition is as disclosed herein but is not a composition comprising both a CAR peptide and another wound therapeutic. As another example, a group of wound healing compositions is contemplated where each composition is as disclosed herein and is able to enhance wound healing.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### B. General

Disclosed are compositions and methods that can be used to enhance wound healing. For example, disclosed are compositions comprising a wound therapeutic, where the only wound therapeutic in the composition is an isolated CAR peptide and/or an isolated truncated CAR peptide. Also disclosed are compositions comprising an isolated peptide, wherein the peptide comprises the amino acid sequence of a CAR peptide and/or a truncated CAR peptide, where the amount of peptide in the composition is a wound healing effective amount. Also disclosed are compositions consisting essentially of an isolated peptide, wherein the C-terminal end of the peptide consists of the amino acid sequence of a CAR peptide and/or a truncated CAR peptide. Also disclosed are compositions comprising an isolated peptide, wherein the C-terminal end of the peptide consists of the amino acid sequence of a CAR peptide and/or a truncated CAR peptide, where the amount of peptide in the composition is a wound healing effective amount. Also disclosed are compositions consisting essentially of an isolated peptide, wherein the C-terminal end of the peptide consists of the amino acid sequence of a CAR peptide and/or a truncated CAR peptide.

Also disclosed are compositions comprising a wound therapeutic, where the only wound therapeutic in the composition is an isolated peptide, wherein the peptide comprises the amino acid sequence CARSKNKDC (SEQ ID NO:10) or the amino acid sequence CARSKNK (SEQ ID NO:4). Also disclosed are compositions comprising an isolated peptide, wherein the peptide comprise the amino acid sequence CARSKNKDC (SEQ ID NO:10) or the amino acid sequence CARSKNK (SEQ ID NO:4), where the amount of peptide in the composition is a wound healing effective amount. Also disclosed are compositions consisting essentially of an isolated peptide, wherein the peptide comprises the amino acid sequence CARSKNKDC (SEQ ID NO:10) or the amino acid sequence CARSKNK (SEQ ID NO:4).

Also disclosed are compositions comprising a wound therapeutic, where the only wound therapeutic in the composition is an isolated peptide, wherein the C-terminal end of the peptide consists of the amino acid sequence CARSKNKDC (SEQ ID NO:10) or the amino acid sequence CARSKNK (SEQ ID NO:4). Also disclosed are compositions comprising an isolated peptide, wherein the C-terminal end of the peptide consists of the amino acid sequence CARSKNKDC (SEQ ID NO:10) or the amino acid sequence CARSKNK (SEQ ID NO:4), where the amount of peptide in the composition is a wound healing effective amount. Also disclosed are compositions consisting essentially of an isolated peptide, wherein the C-terminal end of the peptide consists of the amino acid sequence CARSKNKDC (SEQ ID NO:10) or the amino acid sequence CARSKNK (SEQ ID NO:4).

Also disclosed are methods of enhancing wound healing comprising exposing wounded tissue to one or more of the disclosed compositions, thereby enhancing wound healing. The wounded tissue can be exposed in vivo, ex vivo, or in vitro. For example, tissue to be introduced into a subject can be exposed ex vivo. For example, transplant cells and/or tissue can be exposed in vivo or ex vivo. For example, transplant cells and/or tissue can be exposed prior to, during, and/or following transplantation.

As used herein, a “wound therapeutic” is any compound, composition, moiety, conjugate, molecule, etc. that, by itself, promotes wound healing. For example, a compound that promotes wound healing when used in combination with another compound or composition but that does not promote wound healing when used without any other compound or material is not a wound therapeutic. On the other hand, the combination of that compound with the other compound or composition (which combination promotes wound healing by itself) would be considered a wound therapeutic. This definition allows compositions of CAR peptides but lacking any other wound therapeutic to be distinguished from compositions of CAR peptides and one or more other wound therapeutics.

As used herein, “wound,” wounded tissue,” and like terms include any cells or tissue that is wounded, injured, inflamed, exhibiting inflammatory injury, etc. As used herein, “wound healing” refers to amelioration or repair of a wound or wounded tissue. The natural process of wound healing is well known and that process is encompassed within this definition as well as altered or modified wound healing such as where scar formation is reduced or eliminated, the amelioration or repair occurs more rapidly or with less residual injury, etc.

As used herein, “wound healing effective amount” refers to as an amount of peptide, compound, composition, conjugate, etc. that alone has a measurable positive effect on wound healing. By alone is meant in the absence of any other compound or material such as another wound therapeutic. For example, the wound healing effective amount of CAR peptide is higher than the therapeutically effective amount of CAR peptide when used as a conjugate with another wound therapeutic such as decorin.

In some forms, the peptide can be a modified peptide. In some forms, the peptide can be a methylated peptide. In some forms, the methylated peptide can comprise a methylated amino acid segment. In some forms, the peptide can be N- or C-methylated in at least one position. In some forms, the peptide can be an activatable peptide. In some forms, the amino acid sequence CARSKNKDC (SEQ ID NO:10) or CARSKNK (SEQ ID NO:4) can be blocked by a blocking group. The blocking group can be coupled to the terminal carboxy group, the terminal amino group, the C-terminal amino acid, the N-terminal amino acid, an amino acid in the CAR or tCAR amino acid sequence, or elsewhere in the peptide. In some forms, the amino acid sequence at the C-terminal end of the peptide can be blocked by a blocking group coupled to the terminal carboxy group. In some forms, the blocking group can comprise an amino acid or an amino acid sequence. In some forms, the amino acid sequence CARSKNKDC (SEQ ID NO:10) or the amino acid sequence CARSKNK (SEQ ID NO:4) can be at the C-terminal end of the peptide. In some forms, the amino acid sequence CARSKNKDC (SEQ ID NO:10) or the amino acid sequence CARSKNK (SEQ ID NO:4) can be at the N-terminal end of the peptide. In some forms, the amino acid sequence CARSKNKDC (SEQ ID NO:10) or the amino acid sequence CARSKNK (SEQ ID NO:4) is not at the N- or C-terminal end of the peptide.

In some forms, the peptide can selectively home to regenerating tissue, a site of injury, a surgical site, a site of inflammation, a site of arthritis, pulmonary arterial hypertension lung vasculature, pulmonary arterial hypertension lesions, remodeled pulmonary arteries, or interstitial space of lungs. In some forms, the composition can selectively home to regenerating tissue, a site of injury, a surgical site, a site of inflammation, a site of arthritis, pulmonary arterial hypertension lung vasculature, pulmonary arterial hypertension lesions, remodeled pulmonary arteries, or interstitial space of lungs.

In some forms, the composition can further comprise a carrier, vehicle, or both. In some forms, the composition can further comprise a carrier, a vehicle, a virus, a phage, a viral particle, a phage particle, a viral capsid, a phage capsid, a virus-like particle, a liposome, a micelle, a bead, a nanoparticle, a microparticle, or a combination. In some forms, the composition can further comprise a plurality of copies of the peptide.

In some forms, the peptide can be comprised in a conjugate. In some forms, the conjugate can comprise one or more homing molecules. In some forms, the composition can further comprise one or more homing molecules. In some forms, the homing molecules can selectively home to lung vasculature, regenerating tissue, wounded tissue, pulmonary arterial hypertension lung vasculature, pulmonary arterial hypertension lesions, remodeled pulmonary arteries, interstitial space of lungs, a site of injury, a surgical site, a site of inflammation, or a site of arthritis. In some forms, the homing molecules can be conjugated with a surface molecule. In some forms, one or more of the conjugated homing molecules can be indirectly conjugated to the surface molecule via a linker. In some forms, the composition can further comprise a plurality of linkers. In some forms, at least one of the linkers can comprise polyethylene glycol.

In some forms, the composition can bind inside blood vessels of regenerating tissue, blood vessels of wounded tissue, or lung blood vessels. In some forms, the composition can be internalized in cells. In some forms, the composition can penetrate tissue. In some forms, one or more of the homing molecules can be modified homing molecules. In some forms, one or more of the homing molecules can comprise a methylated homing molecule. In some forms, one or more of the methylated homing molecules can comprise a methylated amino acid segment.

In some forms, the composition can further comprise one or more moieties. In some forms, the moieties can be independently selected from the group consisting of a therapeutic agent, a therapeutic protein, a therapeutic compound, a therapeutic composition, a carrier, a vehicle, a virus, a phage, a viral particle, a phage particle, a viral capsid, a phage capsid, a virus-like particle, a liposome, a micelle, a bead, a nanoparticle, a microparticle, or a combination. In some forms, the composition can further comprise one or more accessory molecules.

In some forms, the composition can have a therapeutic effect. In some forms, the therapeutic effect can comprise a reduction in inflammation, an increase in speed of wound healing, reduction in amounts of scar tissue, decrease in pain, decrease in swelling, or decrease in necrosis. In some forms, the therapeutic effect can comprise pulmonary vasodilation, decrease in pulmonary pressure, anti-coagulation, airway smooth muscle relaxation, increase in glutathione (GSH), decrease in inflammatory immune response, inhibition of thromboxane synthesis, or inhibition of leukotriene synthesis.

In some forms, the wounded tissue can be in a subject. In some forms, the wounded tissue can be exposed to the composition by administering the composition to the subject. In some forms, the composition can penetrate tissue. In some forms, the composition can penetrate lung vasculature, regenerating tissue, wounded tissue, pulmonary arterial hypertension lung vasculature, pulmonary arterial hypertension lesions, remodeled pulmonary arteries, interstitial space of lungs, a site of injury, a surgical site, ex vivo tissue, transplant tissue, ex vivo transplant tissue, a site of inflammation, or a site of arthritis.

In some forms, the composition further comprises a carrier, a vehicle, a virus, a phage, a viral particle, a phage particle, a viral capsid, a phage capsid, a virus-like particle, a liposome, a micelle, a bead, a nanoparticle, a microparticle, or a combination. In some forms, the wounded tissue can be exposed to a plurality of homing molecules. In some forms, the wounded tissue can be exposed to a plurality of compositions.

In some forms, the subject can have a disease or condition. In some forms, the disease can be pulmonary or fibrotic. In some forms, the disease or condition can be pulmonary arterial hypertension (PAH). In some forms, the disease or condition can be an autoimmune disease. In some forms, the disease or condition can be an inflammatory disease.

In some forms, the subject can have one or more sites to be targeted, where the composition homes to one or more of the sites to be targeted. In some forms, the peptide can selectively home to lung vasculature, regenerating tissue, wounded tissue, pulmonary arterial hypertension lung vasculature, pulmonary arterial hypertension lesions, remodeled pulmonary arteries, interstitial space of lungs, a site of injury, a surgical site, a site of inflammation, or a site of arthritis. In some forms, the composition can selectively home to lung vasculature, regenerating tissue, wounded tissue, pulmonary arterial hypertension lung vasculature, pulmonary arterial hypertension lesions, remodeled pulmonary arteries, interstitial space of lungs, a site of injury, a surgical site, a site of inflammation, or a site of arthritis.

In some forms, the composition can enhance internalization, penetration, or both of one or more blood, plasma, or serum components into or through the wounded tissue. A blood component is any molecule, composition, conjugate, cell, etc. found in natural blood. A plasma component is any molecule, composition, conjugate, etc. found in natural plasma. A serum component is any molecule, composition, conjugate, etc. found in natural serum. In some forms, the composition is not administered with any other wound therapeutic.

Disclosed are peptides that target regenerating tissue, wounded tissue, pulmonary tissue, fibrotic tissue, and related tissue, that enhance wound healing, that are readily internalized into adjacent cells, and that extensively penetrate and invade wounded tissue. The disclosed peptides and compositions can also mediate targeting, internalization, and tissue penetration of compounds and compositions coupled to, associated with, conjugated to, or even co-administered with the peptide. Examples of the disclosed peptides include peptides having the sequence CARSKNKDC (CAR; SEQ ID NO:10) or CARSKNK (truncated CAR; tCAR; SEQ ID NO:4) or peptides consisting of CARSKNKDC (SEQ ID NO:10) or CARSKNK (SEQ ID NO:4). For example, disclosed are peptides where the C-terminal end of the peptide consists of the amino acid sequence CARSKNKDC (SEQ ID NO:10) or the amino acid sequence CARSKNK (SEQ ID NO:4), peptides where the N-terminal end of the peptide consists of the amino acid sequence CARSKNKDC (SEQ ID NO:10) or CARSKNK (SEQ ID NO:4), and peptides where neither the N-terminal end of the peptide nor the C-terminal end of the peptide consists of the amino acid sequence CARSKNKDC (SEQ ID NO:10) or CARSKNK (SEQ ID NO:4). The disclosed peptides can be used in and with a variety of compositions and methods to, for example, enhance wound healing.

A class of peptides was recently described that improves drug delivery by increasing penetration of drugs into tissues, such as solid tumors. These peptides contain a C-terminal C-end Rule (CendR) sequence motif (R/K)XX(R/K), which is responsible for cell internalization and tissue penetration activity. CendR peptides contain a cryptic CendR motif that is proteolytically unmasked in certain tissues. Technology has recently been described that provides a way to overcome the limited tissue penetration. CendR peptides induce extravasation and tissue penetration via a mechanism that involves cell internalization (Teesalu et al, 2009; Sugahara et al, 2009, Sugahara et al, 2010). CendR peptides are defined by the presence of the motif R/KXXR/K (X represents any amino acid), which has to be at the C-terminus for the cell- and tissue-penetration activity. The receptor for the CendR motif was shown to be neuropilin 1 (NRP1) (Teesalu et al, 2009).

NRP1 is a modular transmembrane protein previously identified as a receptor for various forms and isoforms of VEGF and members of the class 3 semaphorin family (Takagi et al, 1987; He and Tessier-Lavigne, 1997, Kolodkin et al, 1997; Soker et al, 1998). Neuropilin 2 (NRP2), the second member of the neuropilin family, exhibits sequence and structure homology with NRP1, and shares common ligands NRP-1, VEGFA₁₆₅ among them (Chen et al, 1997; Kolodkin et al, 1997; Glutzman-Poltorak et al, 2000). However, there are also ligands that show selective affinity for one or the other NRP (Chen et al, 1997; Gluzman-Poltorak et al, 2000). Moreover, NRP1 and NRP2 display different expression patterns, with NRP2 (but not NRP1) overexpressed in tumor lymphatics (Caunt et al, 2008). In the CendR pathway, NRP1 appears to be essential for cell internalization and tissue penetration, whereas the role of NRP2 has not been investigated (Teesalu et al, 2009). The disclosed tCAR peptides do not rely on the CendR pathway for cell internalization (Example 3).

Two peptides that selectively target phage to skin and tendon wounds were identified: CARSKNKDC (CAR, SEQ ID NO:10) and CRKDKC (CRK, SEQ ID NO:2). A truncated form of CAR (CARSKNK; tCAR; SEQ ID NO:4) has been discovered that retains and exceeds the properties of the CAR peptide. Unless the context clearly indicates otherwise, reference to “CAR” and “CAR peptide” refers both to full CAR and to truncated CAR. CAR displays homology to heparin-binding sites in various proteins, and binds to cell surface heparan sulfate and heparin. CRK is homologous to a segment in thrombospondin type 1 repeat. Intravenously injected CAR and CRK phage, and the fluorescein-labeled free peptides selectively accumulate at wound sites, partially co-localizing with blood vessels. The CAR peptide shows a preference for early stages of wound healing, whereas the CRK favors wounds at later stages of wound healing. The CAR peptide is internalized into the target cells and delivers the fluorescent label into their nuclei. These results show that the molecular markers in the vasculature of wound tissues change as healing progresses. CAR peptides are described in U.S. Patent Application Publication No. 2009-0036349.

The disclosed CAR peptides can be specific for a particular pathological lesion or an individual tissue. Examples include wounded tissue, diseased lung tissue, and fibrotic tissue. The ability of compositions to penetrate into the extravascular space is a major factor limiting the targeting efficacy of compositions in vivo. A truncated form of the CAR homing peptide mediates highly efficient internalization of phage and free peptides into cells.

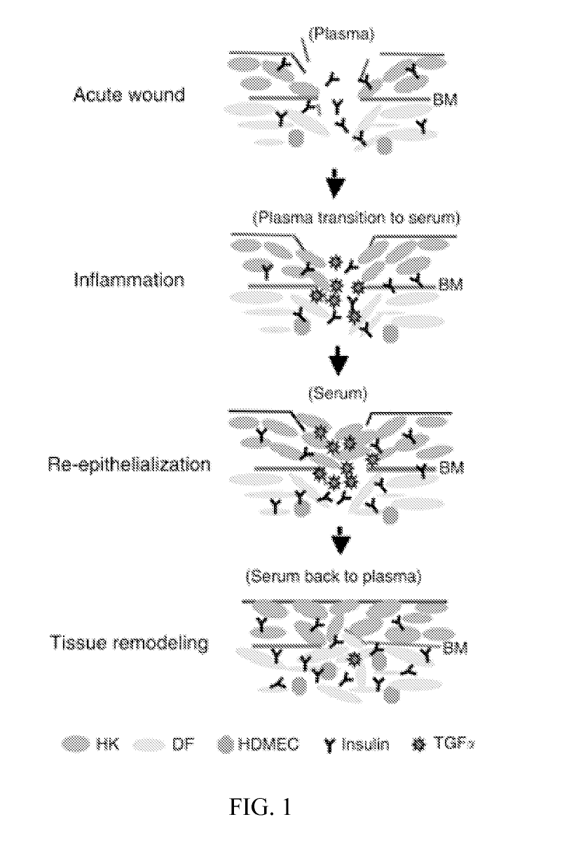

It has now been discovered that CAR peptides alone promote and enhance wound healing. While prior uses of CAR peptides all focused on using the CAR peptide to target therapeutics to homing sites of CAR peptide and/or to promote cell internalization and/or tissue penetration of therapeutics, the use of CAR peptides disclosed herein is based on a newly discovered therapeutic effect of CAR peptides observed in the absence of any other wound therapeutic. CAR penetrates into cells and tissues in a manner that resembles the activity of these CendR peptides (Sugahara et al. 2010). Without wishing to be bound to a particular mechanism of action, it is believed that CAR enhances wound healing by improving the availability to the regenerating tissue of natural growth factors from the blood, plasma, and/or serum, and that because of the wound specificity of CAR, this effect is specific to wounds. According to this scheme, CAR allows pharmacological manipulation of the plasma->serum->plasma transition that takes place during normal tissue repair and controls tissue regeneration (FIG. 1).

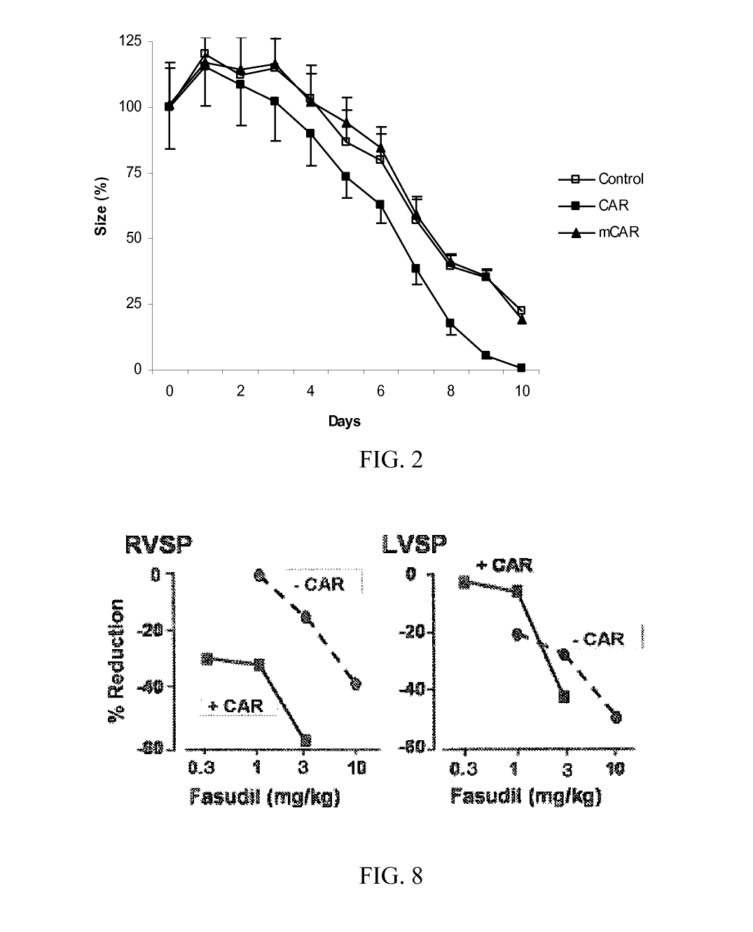

In treatment experiments, intravenous administration of CAR or a control peptide was started on 24 hours after wounding. The treatment was continued for 4, 6 or 9 d in two independent treatment experiments with 15 mice in each treatment group (n=54). CAR was administered in much higher doses than the dose used in targeting CAR-decorin fusion protein to skin wounds (Jarvinen and Ruoslahti, 2010). For example, the molar amount of CAR peptide administered was approximately 40 times the molar amount of CAR peptide administered as CAR-decorin (compare Example 9 with Jarvinen and Ruoslahti, 2010). In prior work (Jarvinen and Ruoslahti, 2010) the dose for CAR-decorin fusions (40 μg) was based on almost entirely on decorin (which dominates the mass of the CAR-decorin fusion) and can be expressed as 1.6 to 2.0 mg fusion/kg subject. CAR peptide was used as a control at the same molar amount as for the CAR-decorin fusion. Thus, the wound healing effective amount of CAR is much higher than the therapeutically effective amount of CAR when used in CAR-decorin compositions. The closure of wounds was significantly accelerated in CAR-treated mice than in controls (FIG. 2, P<0.0001 CAR vs control/mCAR for all time-points from Day 5 on).

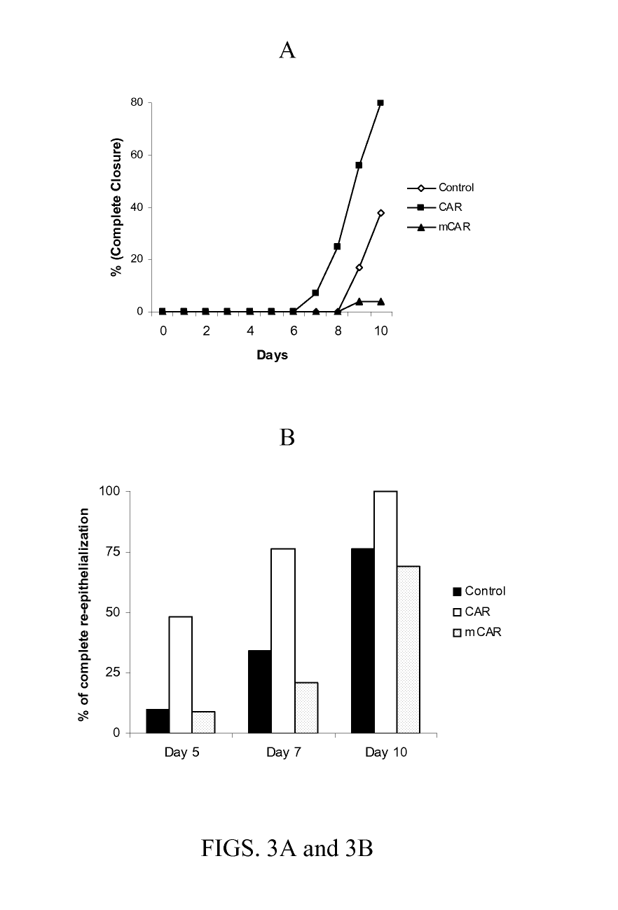

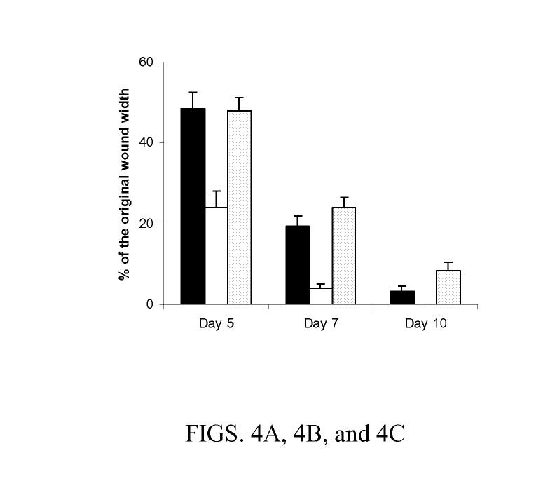

The accelerated wound healing in the CAR-treated mice was also evident when wound closure and re-epithelization was analyzed by assessing the number of wounds that had completely closed/re-epithelialized (FIG. 3). The accelerated wound closure was due to faster re-epithelization of the wounds in the CAR-treated animals as shown in histological sections. Significantly shorter distance between the tips of the epithelial tongues was measured for the CAR-treated wounds than in controls at all time points analyzed (P<0.001, ANOVA, FIG. 4).

In some forms, the peptide and the composition can comprise a therapeutic agent, a therapeutic protein, a therapeutic compound, a therapeutic composition, an anti-inflammatory agent, an anti-arthritic agent, a TGF-β inhibitor, decorin, a systemic vasodilator, an anti-coagulant, tissue factor pathway inhibitor (TFPI), site-inactivated factor VIIa, a β-2 agonist, salmeterol, formoterol, N-Acetylcysteine (NAC), Superoxide Dismutase (SOD), a superoxide dismutase mimetic, EUK-8, an endothelin (ET-1) receptor antagonist, a prostacyclin derivative, a phosphodiesterase type 5 inhibitor, Ketoconazole, a toxin, a cytotoxic agent, an anti-angiogenic agent, a pro-angiogenic agent, an antibody, a small interfering RNA (siRNA), a microRNA (miRNA), a polypeptide, a nucleic acid molecule, a small molecule, a carrier, a vehicle, a virus, a phage, a viral particle, a phage particle, a viral capsid, a phage capsid, a virus-like particle, a liposome, a micelle, a bead, a nanoparticle, a microparticle, a detectable agent, a contrast agent, an imaging agent, a label, a labeling agent, a fluorophore, fluorescein, rhodamine, FAM, a radionuclide, indium-111, technetium-99, carbon-11, carbon-13, or a combination.

In some forms, the surface molecule can comprise a nanoparticle, a nanoworm, an iron oxide nanoworm, an iron oxide nanoparticle, an albumin nanoparticle, a liposome, a micelle, a phospholipid, a polymer, a microparticle, or a fluorocarbon microbubble. In some forms, the composition can comprise at least 100 homing molecules, at least 1000 homing molecules, or at least 10,000 homing molecules. In some forms, the composition can comprise at least 100 membrane perturbing molecules, at least 1000 membrane perturbing molecules, or at least 10,000 membrane perturbing molecules.

In some forms, one or more of the homing molecules can be modified homing molecules. In some forms, one or more of the homing molecules can comprise a methylated homing molecule. In some forms, one or more of the methylated homing molecules can comprise a methylated amino acid segment. In some forms, one or more of the membrane perturbing molecules can be modified membrane perturbing molecules. In some forms, one or more of the membrane perturbing molecules can comprise a methylated membrane perturbing molecule. In some forms, one or more of the methylated membrane perturbing molecules can comprise a methylated amino acid segment. In some forms, the amino acid segment can be N- or C-methylated in at least one position.

In some forms, the composition can further comprise one or more moieties. In some forms, the moieties can be independently selected from the group consisting of a therapeutic agent, a therapeutic protein, a therapeutic compound, a therapeutic composition, an anti-inflammatory agent, an anti-arthritic agent, a TGF-β inhibitor, decorin, a systemic vasodilator, an anti-coagulant, tissue factor pathway inhibitor (TFPI), site-inactivated factor VIIa, a β-2 agonist, salmeterol, formoterol, N-Acetylcysteine (NAC), Superoxide Dismutase (SOD), a superoxide dismutase mimetic, EUK-8, an endothelin (ET-1) receptor antagonist, a prostacyclin derivative, a phosphodiesterase type 5 inhibitor, Ketoconazole, a toxin, a cytotoxic agent, an anti-angiogenic agent, a pro-angiogenic agent, an antibody, a small interfering RNA (siRNA), a microRNA (miRNA), a polypeptide, a nucleic acid molecule, a small molecule, a carrier, a vehicle, a virus, a phage, a viral particle, a phage particle, a viral capsid, a phage capsid, a virus-like particle, a liposome, a micelle, a bead, a nanoparticle, a microparticle, a detectable agent, a contrast agent, an imaging agent, a label, a labeling agent, a fluorophore, fluorescein, rhodamine, FAM, a radionuclide, indium-111, technetium-99, carbon-11, carbon-13, or a combination.

In some forms, the composition can have a therapeutic effect. In some forms, the therapeutic effect can comprise a reduction in inflammation, an increase in speed of wound healing, reduction in amounts of scar tissue, decrease in pain, decrease in swelling, or decrease in necrosis. In some forms, the therapeutic effect can comprise pulmonary vasodilation, decrease in pulmonary pressure, anti-coagulation, airway smooth muscle relaxation, increase in glutathione (GSH), decrease in inflammatory immune response, inhibition of thromboxane synthesis, or inhibition of leukotriene synthesis.

In some forms, the composition can penetrate tissue. In some forms, the composition can penetrate lung vasculature, regenerating tissue, wounded tissue, angiogenic tissue, lung tissue, pulmonary arterial hypertension lung vasculature, pulmonary arterial hypertension lesions, remodeled pulmonary arteries, interstitial space of lungs, a site of injury, a surgical site, ex vivo tissue, transplant tissue, ex vivo transplant tissue, a site of angiogenesis, a site of inflammation, or a site of arthritis.

In some forms, the composition can comprise one or more accessory molecules. In some forms, the cell, tissue, or both can be exposed to a plurality of homing molecules. In some forms, the cell, tissue, or both can be exposed to a plurality of compositions.

In some forms, the subject has a disease or condition. In some forms, the disease can be pulmonary or fibrotic. In some forms, the disease or condition can be pulmonary arterial hypertension (PAH). In some forms, the disease or condition can be an autoimmune disease. In some forms, the disease or condition can be an inflammatory disease. In some forms, the condition can be cuts and/or abrasions. In some forms, the condition can be burns. In some forms, the condition can be ischemia, such as caused by stokes and blood clots.

In some forms, the subject can have one or more sites to be targeted, wherein the composition homes to one or more of the sites to be targeted. In some forms, the peptide can selectively home to lung vasculature, regenerating tissue, wounded tissue, angiogenic tissue, lung tissue, pulmonary arterial hypertension lung vasculature, pulmonary arterial hypertension lesions, remodeled pulmonary arteries, interstitial space of lungs, a site of injury, a surgical site, a site of angiogenesis, a site of inflammation, or a site of arthritis.

In some forms, the composition can selectively home to lung vasculature, regenerating tissue, wounded tissue, angiogenic tissue, lung tissue, pulmonary arterial hypertension lung vasculature, pulmonary arterial hypertension lesions, remodeled pulmonary arteries, interstitial space of lungs, a site of injury, a surgical site, a site of angiogenesis, a site of inflammation, or a site of arthritis.

The disease can be selected from the group consisting of pulmonary hypertension, interstitial lung disease, acute lung injury (ALI), acute respiratory distress syndrome (ARDS), sepsis, septic shock, sarcoidosis of the lung, pulmonary manifestations of connective tissue diseases, including systemic lupus erythematosus, rheumatoid arthritis, scleroderma, and polymyositis, dermatomyositis, bronchiectasis, asbestosis, berylliosis, silicosis, Histiocytosis X, pneumotitis, smoker's lung, bronchiolitis obliterans, the prevention of lung scarring due to tuberculosis and pulmonary fibrosis, other fibrotic diseases such as myocardial infarction, endomyocardial fibrosis, mediastinal fibrosis, myelofibrosis, retroperitoneal fibrosis, progressive massive fibrosis, pneumoconiosis, nephrogenic systemic fibrosis, keloid, arthrofibrosis, adhesive capsulitis, radiation fibrosis, fibrocystic breast condition, liver cirrhosis, hepatitis, liver fibrosis, nonalcoholic fatty liver disease, nonalcoholic steatohepatitis, sarcoidosis of the lymph nodes, or other organs, inflammatory bowel disease, crohn's disease, ulcerative colitis, primary biliary cirrhosis, pancreatitis, interstitial cystitis, chronic obstructive pulmonary disease, pneumoconiosis, autoimmune diseases, angiogenic diseases, wound healing, infections, trauma injuries and systemic connective tissue diseases including systemic lupus erythematosus, rheumatoid arthritis, scleroderma, polymyositis, and dermatomyositis.

Disclosed are CAR compositions, CAR conjugates, CAR molecules, CAR proteins, and CAR peptides. CAR peptides are the basic feature of CAR compositions, CAR conjugates, CAR molecules, CAR proteins, and the like. CAR compositions are any composition, conglomeration, conjugate, molecule, protein, peptide, etc. that comprises a CAR peptide. CAR conjugates are associations, whether covalent or non-covalent, of a CAR peptide and one or more other elements, peptides, proteins, compounds, molecules, agents, compounds, etc. For example, a CAR conjugate can comprise a CAR peptide, CAR protein, CAR compound, CAR molecule, etc. CAR molecules are molecules that comprise a CAR peptide. For example, a CAR molecule can comprise a CAR protein, CAR peptide, etc. In general, CAR peptides, CAR proteins, CAR molecules, and CAR conjugates are all forms of CAR compositions. CAR compounds, CAR peptides and CAR proteins can be forms of CAR molecules. Unless the context indicates otherwise, reference to a CAR composition is intended to refer to CAR compositions, CAR molecules, CAR proteins, CAR peptides, and the like. A CAR component is any molecule, peptide, protein, compound, conjugate, composition, etc. that comprises a CAR peptide. Examples of CAR components include, for example, CAR compositions, CAR molecules, CAR proteins, and CAR peptides.

CAR components can comprise one or more CAR peptides. Where a CAR element comprises two or more CAR peptides, it is useful for the CAR component to be designed to allow some or all of the CAR peptides to be exposed at the C-terminus of a protein or peptide. This can be accomplished in numerous ways in, for example, conjugates and compositions. This can also be accomplished in, for example, branching peptides and proteins.

Disclosed are peptides that promote and enhance wound healing and that target wounded tissue, regenerating tissue, sites of injury, surgical sites, sites of angiogenesis, sites of inflammation, sites of arthritis, lung tissue, pulmonary arterial hypertension lung vasculature, pulmonary arterial hypertension lesions, remodeled pulmonary arteries, and interstitial space of lungs, are readily internalized into adjacent cells, and extensively penetrate and invade the targeted tissue. The disclosed peptides can also mediate targeting, internalization, and tissue penetration of compounds and compositions coupled to, associated with, conjugated to, or even co-administered with the peptide. Examples of the disclosed peptides include peptides having the sequence CARSKNKDC (CAR; SEQ ID NO:10) or CARSKNK (truncated CAR; tCAR; SEQ ID NO:4) or peptides consisting of CARSKNKDC (SEQ ID NO:10) or CARSKNK (SEQ ID NO:4). For example, disclosed are peptides where the C-terminal end of the peptide consists of the amino acid sequence CARSKNKDC (SEQ ID NO:10) or the amino acid sequence CARSKNK (SEQ ID NO:4), peptides where the N-terminal end of the peptide consists of the amino acid sequence CARSKNKDC (SEQ ID NO:10) or CARSKNK (SEQ ID NO:4), and peptides where neither the N-terminal end of the peptide nor the C-terminal end of the peptide consists of the amino acid sequence CARSKNKDC (SEQ ID NO:10) or CARSKNK (SEQ ID NO:4). The disclosed peptides can be used in and with a variety of compositions and methods to, for example, promoting and enhancing wound healing, and enhancing internalization, penetration, or both of such compositions into or through a cell, tissue, or both. Such compositions and methods are also disclosed herein.

Disclosed are peptides comprising the amino acid sequence CARSKNKDC (SEQ ID NO:10) or the amino acid sequence CARSKNK (SEQ ID NO:4). Also disclosed are peptides where the C-terminal end of the peptide consists of the amino acid sequence CARSKNKDC (SEQ ID NO:10) or the amino acid sequence CARSKNK (SEQ ID NO:4). In some forms, the peptide can be a modified peptide. In some forms, the peptide can be a methylated peptide. In some forms, one or more of the methylated peptide can comprise a methylated amino acid segment. In some forms, the peptide can be N- or C-methylated in at least one position.

CAR peptides are peptides consisting of or having the amino acid sequence CARSKNKDC (SEQ ID NO:10) the amino acid sequence CARSKNK (SEQ ID NO:4). Generally, CAR peptides do not comprise the amino acid sequence CARSKNKDC (SEQ ID NO:10). CAR peptides can be composed of standard amino acids with standard peptide linkages or can be embodied in other than standard amino acids and/or with other than standard peptide linkages. CAR peptides can include modifications to the peptide, amino acids, and/or linkages. Examples of suitable modifications known to those in the art and are described elsewhere herein. Variant CAR peptides can be used in place of or in addition to CAR peptides. Variant CAR peptides are not CAR peptides.

Pulmonary arterial hypertension (PAH) is a disorder of the pulmonary vasculature associated with elevated pulmonary vascular resistance. Despite recent advances in the treatment of PAH, with eight approved clinical therapies and additional therapies undergoing clinical trials, PAH remains a serious, life-threatening condition. The lack of pulmonary vascular selectivity and associated systemic adverse effects of these therapies remain the main obstacles to successful treatment. Highly selective targeting of rat PAH lesions by CAR peptide can aid healing of the tissue injuries in PAH. Intravenous administration of CAR peptide resulted in intense accumulation of the peptide in monocrotaline-induced and SU5416/hypoxia-induced hypertensive lungs but not in the normal healthy lungs or in other organs of the PAH rats. CAR homed to all layers of remodeled pulmonary arteries, i.e. endothelium, neointima, medial smooth muscle, and adventitia, in the hypertensive lungs. CAR also homed to capillary vessels and accumulated in the interstitial space of the PAH lungs, manifesting its extravasation activity. The results demonstrated a remarkable ability of CAR to selectively target PAH lung vasculature, effectively penetrate, and spread throughout the diseased lung tissue. These results indicate clinical utility of CAR in treating PAH.

In some forms, one or more of the homing molecules can comprise the amino acid sequence CGKRK (SEQ ID NO:1) or a conservative derivative thereof, the amino acid sequence CRKDKC (SEQ ID NO:2) or a conservative derivative thereof, or a combination. In some forms, one or more of the homing molecules can comprise the amino acid sequence CGKRK (SEQ ID NO:1) or a conservative variant thereof. In some forms, one or more of the homing molecules can comprise the amino acid sequence CGKRK (SEQ ID NO:1). In some forms, all of the one or more homing molecules can comprise the amino acid sequence CGKRK (SEQ ID NO:1) or a conservative derivative thereof, the amino acid sequence CRKDKC (SEQ ID NO:2) or a conservative derivative thereof, or a combination.

In some forms, one or more of the membrane perturbing molecules can comprise the amino acid sequence _{D}(KLAKLAK)₂ (SEQ ID NO:3) or a conservative variant thereof, (KLAKLAK)₂ (SEQ ID NO:3) or a conservative variant thereof, (KLAKKLA)₂ (SEQ ID NO:5) or a conservative variant thereof, (KAAKKAA)₂ (SEQ ID NO:6) or a conservative variant thereof, or (KLGKKLG)₃ (SEQ ID NO:7) or a conservative variant thereof, or a combination. In some forms, one or more of the membrane perturbing molecules can comprise the amino acid sequence _{D}(KLAKLAK)₂ (SEQ ID NO:3), (KLAKLAK)₂ (SEQ ID NO:3), (KLAKKLA)₂ (SEQ ID NO:5), (KAAKKAA)₂ (SEQ ID NO:6), or (KLGKKLG)₃ (SEQ ID NO:7), or a combination. In some forms, one or more of the membrane perturbing molecules can comprise the amino acid sequence _{D}(KLAKLAK)₂ (SEQ ID NO:3) or a conservative variant thereof. In some forms, one or more of the membrane perturbing molecules can comprise the amino acid sequence _{D}(KLAKLAK)₂ (SEQ ID NO:3).

In some forms, one or more of the membrane perturbing molecules can be conjugated to one or more of the homing molecules. In some forms, the homing molecules can be conjugated with the surface molecule. In some forms, the membrane perturbing molecules can be conjugated with the surface molecule. In some forms, one or more of the conjugated homing molecules can be indirectly conjugated to the surface molecule via a linker, one or more of the conjugated membrane perturbing molecules can be indirectly conjugated to the surface molecule via a linker, or both. In some forms, the composition can further comprise a plurality of linkers. In some forms, at least one of the linkers can comprise polyethylene glycol.

In some forms, the surface molecule can comprise a nanoparticle, a nanoworm, an iron oxide nanoworm, an iron oxide nanoparticle, an albumin nanoparticle, a liposome, a micelle, a phospholipid, a polymer, a microparticle, or a fluorocarbon microbubble. In some forms, the composition can comprise at least 100 homing molecules. In some forms, the composition can comprise at least 1000 homing molecules. In some forms, the composition can comprise at least 10,000 homing molecules. In some forms, the composition can comprise at least 100 membrane perturbing molecules. In some forms, the composition can comprise at least 1000 membrane perturbing molecules. In some forms, the composition can comprise at least 10,000 membrane perturbing molecules. In some forms, the composition can comprise at least 100 copies of the peptide. In some forms, the composition can comprise at least 1000 copies of the peptide. In some forms, the composition can comprise at least 10,000 copies of the peptide.

In some forms, the composition can comprise a plurality of surface molecules and/or a plurality of homing molecules. In some forms, the composition can comprise one or more surface molecules and/or a plurality of homing molecules. In some forms, the composition can comprise a plurality of surface molecules and/or one or more homing molecules. In some forms, the composition can comprise one or more surface molecules and/or one or more homing molecules.

In some forms, the composition can comprise a surface molecule and a plurality of homing molecules, wherein one or more of the homing molecules are associated with the surface molecule. In some forms, the composition can comprise a surface molecule and a plurality of homing molecules, wherein a plurality of the plurality of homing molecules are associated with the surface molecule. In some forms, the composition can comprise a surface molecule and a plurality of homing molecules and a plurality of cargo molecules, wherein the homing molecules are associated with the surface molecule.

In some forms, the composition can comprise a surface molecule, wherein the surface molecule is multivalent for homing molecules. In some forms, the composition can comprise a surface molecule, wherein the surface molecule is multivalent for conjugates, wherein one or more of the conjugates comprise one or more homing molecules. In some forms, the composition can comprise a surface molecule, wherein the surface molecule is multivalent for conjugates, wherein one or more of the conjugates comprise a plurality of homing molecules. In some forms, the composition can comprise a surface molecule, wherein the surface molecule is multivalent for conjugates, wherein one or more of the conjugates comprise a homing molecule. In some forms, the composition can comprise a surface molecule, wherein the surface molecule is multivalent for conjugates, wherein each of the conjugates comprises a plurality of homing molecules. In some forms, the composition can comprise a surface molecule, wherein the surface molecule is multivalent for conjugates, wherein each of the conjugates comprises a homing molecule. As used herein, a component that is stated to be “multivalent for” one or more other components refers to a component that has a plurality of the other components associated with, conjugated to and/or covalent coupled to the first component.

In some forms, the composition, can comprise a surface molecule, wherein the surface molecule comprises one or more conjugates, wherein one or more of the conjugates comprise one or more homing molecules. In some forms, the composition can comprise a surface molecule, wherein the surface molecule comprises one or more conjugates, wherein one or more of the conjugates comprise a plurality of homing molecules. In some forms, the composition can comprise a surface molecule, wherein the surface molecule comprises one or more conjugates, wherein one or more of the conjugates comprise a homing molecule. In some forms, the composition can comprise a surface molecule, wherein the surface molecule comprises one or more conjugates, wherein each of the conjugates comprises a plurality of homing molecules. In some forms, the composition can comprise a surface molecule, wherein the surface molecule comprises one or more conjugates, wherein each of the conjugates comprises a homing molecule.

In some forms, one or more of the membrane perturbing molecules can be conjugated to one or more of the homing molecules. In some forms, one or more of the conjugated membrane perturbing molecules and homing molecules can be covalently coupled. In some forms, one or more of the covalently coupled membrane perturbing molecules and homing molecules can comprise fusion peptides. In some forms, the homing molecules can be conjugated with the surface molecule. In some forms, one or more of the conjugated homing molecules can be directly conjugated to the surface molecule. In some forms, one or more of the conjugated homing molecules can be indirectly conjugated to the surface molecule. In some forms, one or more of the homing molecules can be covalently coupled to the surface molecule. In some forms, one or more of the covalently coupled homing molecules can be directly covalently coupled to the surface molecule. In some forms, one or more of the covalently coupled homing molecules can be indirectly covalently coupled to the surface molecule. In some forms, the membrane perturbing molecules can be conjugated with the surface molecule. In some forms, one or more of the conjugated membrane perturbing molecules are directly conjugated to the surface molecule. In some forms, one or more of the conjugated membrane perturbing molecules can be indirectly conjugated to the surface molecule. In some forms, one or more of the membrane perturbing molecules can be covalently coupled to the surface molecule. In some forms, one or more of the covalently coupled membrane perturbing molecules can be directly covalently coupled to the surface molecule. In some forms, one or more of the covalently coupled membrane perturbing molecules can be indirectly covalently coupled to the surface molecule.

In some forms, the surface molecule can comprise a nanoparticle. In some forms, the surface molecule can comprise a nanoworm. In some forms, the surface molecule can comprise an iron oxide nanoworm. In some forms, the surface molecule can comprise an iron oxide nanoparticle. In some forms, the surface molecule can comprise an albumin nanoparticle. In some forms, the surface molecule can comprise a liposome. In some forms, the surface molecule can comprise a micelle. In some forms, the surface molecule comprises a phospholipid. In some forms, the surface molecule comprises a polymer. In some forms, the surface molecule can comprise a microparticle. In some forms, the surface molecule can comprise a fluorocarbon microbubble.

In some forms, the composition can comprise at least 100 homing molecules. In some forms, the composition, can comprise at least 1000 homing molecules. In some forms, the composition can comprise at least 10,000 homing molecules. In some forms, the composition can comprise at least 100 membrane perturbing molecules. In some forms, the composition can comprise at least 1000 membrane perturbing molecules. In some forms, the composition can comprise at least 10,000 membrane perturbing molecules. In some forms, the composition can comprise at least 100 CAR peptides. In some forms, the composition can comprise at least 1000 CAR peptides. In some forms, the composition can comprise at least 10,000 CAR peptides.

In some forms, one or more of the homing molecules can be modified homing molecules. In some forms, one or more of the homing molecules can comprise a methylated homing molecule. In some forms, one or more of the methylated homing molecules can comprise a methylated amino acid segment. In some forms, one or more of the membrane perturbing molecules can be modified membrane perturbing molecules. In some forms, one or more of the membrane perturbing molecules can comprise a methylated membrane perturbing molecule. In some forms, one or more of the methylated membrane perturbing molecules can comprise a methylated amino acid segment. In some forms, the amino acid segment can be N- or C-methylated in at least one position.

In some forms, the composition can further comprise one or more moieties. In some forms, the moieties can be independently selected from the group consisting of, for example, a therapeutic agent, a therapeutic protein, a therapeutic compound, a therapeutic composition, an anti-inflammatory agent, an anti-arthritic agent, a TGF-β inhibitor, decorin, a systemic vasodilator, an anti-coagulant, tissue factor pathway inhibitor (TFPI), site-inactivated factor VIIa, a β-2 agonist, salmeterol, formoterol, N-Acetylcysteine (NAC), Superoxide Dismutase (SOD), a superoxide dismutase mimetic, EUK-8, an endothelin (ET-1) receptor antagonist, a prostacyclin derivative, a phosphodiesterase type 5 inhibitor, Ketoconazole, a toxin, a cytotoxic agent, an anti-angiogenic agent, a pro-angiogenic agent, an antibody, a small interfering RNA (siRNA), a microRNA (miRNA), a polypeptide, a nucleic acid molecule, a small molecule, a carrier, a vehicle, a virus, a phage, a viral particle, a phage particle, a viral capsid, a phage capsid, a virus-like particle, a liposome, a micelle, a bead, a nanoparticle, a microparticle, a detectable agent, a contrast agent, an imaging agent, a label, a labeling agent, a fluorophore, fluorescein, rhodamine, FAM, a radionuclide, indium-111, technetium-99, carbon-11, and carbon-13. In some forms, at least one of the moieties can be a therapeutic agent. In some forms, at least one of the moieties can be a detectable agent. In some forms, the detectable agent can be FAM.

The term “bioactive agent” refers to a substance which is used in connection with an application that is therapeutic or diagnostic in nature, such as in methods for diagnosing the presence or absence of a disease in a patient and/or in methods for treating a disease in a patient. Therapeutic agents and detectable agents are examples of bioactive agents. As to compatible bioactive agents, those skilled in the art will appreciate that any therapeutic or diagnostic agent may be incorporated in the stabilized dispersions of the disclosed compositions. For example, the bioactive agent may be selected from the group consisting of antiallergics, bronchodilators, vasodilators, antihypertensive agents, bronchoconstrictors, pulmonary lung surfactants, analgesics, antibiotics, leukotriene inhibitors or antagonists, anticholinergics, mast cell inhibitors, antihistamines, anti-inflammatories, anti-neoplastics, anesthetics, anti-tuberculars, imaging agents, cardiovascular agents, enzymes, steroids, genetic material, viral vectors, antisense agents, small molecule drugs, proteins, peptides and combinations thereof. Particularly preferred bioactive agents comprise compounds which are to be administered systemically (i.e. to the systemic circulation of a patient) such as small molecule drugs, imaging agents, peptides, proteins or polynucleotides. As is disclosed in more detail elsewhere herein, the bioactive agent can be incorporated, blended in, coated on or otherwise associated with the targeting peptide disclosed herein. Particularly preferred bioactive agents for use in the disclosed compositions and methods include anti-allergics, peptides and proteins, bronchodilators, anti-inflammatory agents for use in the treatment of disorders involving diseased tissue reflecting altered heparan sulfate variants specific to said disease. Yet another associated advantage of the disclosed compositions and methods is the effective delivery of bioactive agents administered or combined with a targeting peptide.

In some forms, one or more of the homing molecules can comprise the amino acid sequence CGKRK (SEQ ID NO:1), where one or more of the membrane perturbing molecules can comprise the amino acid sequence _{D}(KLAKLAK)₂ (SEQ ID NO:3), where one or more of the homing molecules can be indirectly conjugated to the surface molecule via a linker, and where one or more of the membrane perturbing molecules can be indirectly conjugated to the surface molecule via a linker. In some forms, at least one of the linkers can comprise polyethylene glycol.

The composition can comprise any of the disclosed CAR peptides or any of the disclosed compositions that comprise a CAR peptide.

Also disclosed are methods of enhancing wound healing by exposing wounded tissue to one or more of the disclosed compositions, thereby enhancing wound healing. In some forms, the composition can enhance internalization, penetration, or both of one or more blood, plasma, or serum components into or through the wounded tissue. In some forms, the composition is not administered with any other wound therapeutic. Because CAR peptides alone can have a beneficial effect, it is not required to include or administer any other wound therapeutic. However, the disclosed CAR peptides, CAR compositions, CAR conjugates, etc. can be administered together with, at the same time as, and/or during the same or overlapping treatment periods as other therapeutics that are not wound therapeutics.

As used herein, “administered with, “administered together with,” and like terms means that one component is administered in the same composition as another component. As used herein, “administered at the same time as” and like terms means that one component is administered at the same time as another component. By at the same time is meant simultaneously and/or overlapping in time, and/or As used herein, “administered during the same treatment period,” “administered during overlapping treatment periods,” or like terms means that one component is administered during the period when the other component remains therapeutically effective. The period when a component remains therapeutically effective refers to the period before the component is turned over, cleared, broken down, altered, etc. to a sub-therapeutic amount or concentration.

In some forms, the composition can be administered prior to a wound or injury. For example, a subject expected to or at risk to suffer an injury, such as a subject about to undergo surgery, can be treated prior to or during the injury. In some forms, the composition can be administered at the same time as an injury. In some forms, the composition can be administered soon after an injury. For example, the composition can be administered 1, 2, 3, 4, 5, 6, 7, 8, 9, 10, 15, 20, 25, 30, 35, 40, 45, 50, 55, 60, 65, 70, 75, 80, 85, 90, 95, 100, 120, 140, 150, 160, 180, 200, 220, 240, 250, 260, 280, 300, 350, 400, 450, 500, 600, 700, 800, 900, 1000, 1100, 1200, 1300, 1400, or 1500 minutes following an injury or the beginning of an injury. For example, the composition can be administered 1, 2, 3, 4, 5, 6, 7, 8, 9, 10, 15, 20, 25, 30, 35, 40, 45, 50, 55, 60, 65, 70, 75, 80, 85, 90, 95, 100, 120, 140, 150, 160, 180, 200, 220, 240, 250, 260, 280, 300, 350, 400, 450, 500, 600, 700, 800, 900, 1000, 1100, 1200, 1300, 1400, 1500 or more minutes following an injury or the beginning of an injury. For example, the composition can be administered at least 1, 2, 3, 4, 5, 6, 7, 8, 9, 10, 15, 20, 25, 30, 35, 40, 45, 50, 55, 60, 65, 70, 75, 80, 85, 90, 95, 100, 120, 140, 150, 160, 180, 200, 220, 240, 250, 260, 280, 300, 350, 400, 450, 500, 600, 700, 800, 900, 1000, 1100, 1200, 1300, 1400, or 1500 minutes following an injury or the beginning of an injury. For example, the composition can be administered within 1, 2, 3, 4, 5, 6, 7, 8, 9, 10, 15, 20, 25, 30, 35, 40, 45, 50, 55, 60, 65, 70, 75, 80, 85, 90, 95, 100, 120, 140, 150, 160, 180, 200, 220, 240, 250, 260, 280, 300, 350, 400, 450, 500, 600, 700, 800, 900, 1000, 1100, 1200, 1300, 1400, or 1500 minutes following an injury or the beginning of an injury. For example, the composition can be administered about 1, 2, 3, 4, 5, 6, 7, 8, 9, 10, 15, 20, 25, 30, 35, 40, 45, 50, 55, 60, 65, 70, 75, 80, 85, 90, 95, 100, 120, 140, 150, 160, 180, 200, 220, 240, 250, 260, 280, 300, 350, 400, 450, 500, 600, 700, 800, 900, 1000, 1100, 1200, 1300, 1400, or 1500 minutes following an injury or the beginning of an injury.

For example, the composition can be administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 hours following an injury or the beginning of an injury. For example, the composition can be administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more hours following an injury or the beginning of an injury. For example, the composition can be administered at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 hours following an injury or the beginning of an injury. For example, the composition can be administered within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 hours following an injury or the beginning of an injury. For example, the composition can be administered about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 hours following an injury or the beginning of an injury.

For example, the composition can be administered 1, 2, 3, 4, 5, 6, 7, 8, 9, 10, 11, 12, 13, 14, 15, 16, 17, 18, 19, or 20 days following an injury or the beginning of an injury. For example, the composition can be administered 1, 2, 3, 4, 5, 6, 7, 8, 9, 10, 11, 12, 13, 14, 15, 16, 17, 18, 19, 20 or more days following an injury or the beginning of an injury. For example, the composition can be administered at least 1, 2, 3, 4, 5, 6, 7, 8, 9, 10, 11, 12, 13, 14, 15, 16, 17, 18, 19, or 20 days following an injury or the beginning of an injury. For example, the composition can be administered within 1, 2, 3, 4, 5, 6, 7, 8, 9, 10, 11, 12, 13, 14, 15, 16, 17, 18, 19, or 20 days following an injury or the beginning of an injury. For example, the composition can be administered about 1, 2, 3, 4, 5, 6, 7, 8, 9, 10, 11, 12, 13, 14, 15, 16, 17, 18, 19, or 20 days following an injury or the beginning of an injury.

In some forms, the wounded tissue can be in a subject. In some forms, the wounded tissue can be exposed to the composition by administering the composition to the subject. In some forms, the composition can penetrate tissue. In some forms, the composition can penetrate lung vasculature, regenerating tissue, wounded tissue, pulmonary arterial hypertension lung vasculature, pulmonary arterial hypertension lesions, remodeled pulmonary arteries, interstitial space of lungs, a site of injury, a surgical site, ex vivo tissue, transplant tissue, ex vivo transplant tissue, a site of inflammation, or a site of arthritis.

In some forms, the composition further comprises a carrier, a vehicle, a virus, a phage, a viral particle, a phage particle, a viral capsid, a phage capsid, a virus-like particle, a liposome, a micelle, a bead, a nanoparticle, a microparticle, or a combination. In some forms, the wounded tissue can be exposed to a plurality of homing molecules. In some forms, the wounded tissue can be exposed to a plurality of compositions.

In some forms, the subject can have a disease or condition. In some forms, the disease can be pulmonary or fibrotic. In some forms, the disease or condition can be pulmonary arterial hypertension (PAH). In some forms, the disease or condition can be an autoimmune disease. In some forms, the disease or condition can be an inflammatory disease.

In some forms, the subject can have one or more sites to be targeted, where the composition homes to one or more of the sites to be targeted. In some forms, the peptide can selectively home to lung vasculature, regenerating tissue, wounded tissue, pulmonary arterial hypertension lung vasculature, pulmonary arterial hypertension lesions, remodeled pulmonary arteries, interstitial space of lungs, a site of injury, a surgical site, a site of inflammation, or a site of arthritis. In some forms, the composition can selectively home to lung vasculature, regenerating tissue, wounded tissue, pulmonary arterial hypertension lung vasculature, pulmonary arterial hypertension lesions, remodeled pulmonary arteries, interstitial space of lungs, a site of injury, a surgical site, a site of inflammation, or a site of arthritis.

Also disclosed are methods of enhancing internalization, penetration, or both into or through a cell, tissue, or both. In some forms, the method can comprise exposing the cell, tissue, or both to a CAR composition, thereby enhancing internalization, penetration, or both into or through the cell, tissue, or both. The CAR composition can comprise any of the disclosed CAR peptides or any of the disclosed compositions that comprise a CAR peptide.

In some forms, the cell, tissue, or both can be in a subject. In some forms, the cell, tissue, or both can be exposed to the composition by administering the composition to the subject. In some forms, the cell, tissue, or both can be exposed to the CAR composition by administering the CAR composition to the subject.

When tissue is injured, there is an inflammatory phase (up to day 3) before revascularization begins. When injured tissue is revascularized, sprouting angiogenesis starts at day 3, peaks between days 5 to 7, after which most of the tiny capillaries regress and those few ones remaining mature into more stable vessels. It has been shown (U.S. Patent Application Publication No. 2009-0036349) that the CAR peptide favors the early sprouting, angiogenic blood vessels, whereas the CRK peptide shows preferences for the mature blood vessels at the later stages of healing. Now it has been discovered that the CAR peptide can effectively home to and target injured tissue during the inflammatory phase (FIG. 9).

Based on this discovery, the disclosed compositions and methods can be used to target wounded tissue before or immediately after (0 to 3 days) an injury. The inflammatory phase of the healing process starts by bleeding from the ruptured blood vessels followed by clotting, that is, the coagulation of the blood (FIG. 1). The ruptured blood vessels are contracted during the bleeding to reduce the amount of blood lost, but after the coagulation (4 to 6 hours later) they dilate and the inflammatory cells start extravasating from the existing blood vessels to the injured area (the blood vessels do not leak). This is an active process, where the normal blood vessels next to the injured area are “primed”/“activated” and recognized by the inflammatory cells. By recognizing the “cues” on the blood vessels next to the site of injury, the inflammatory cells can bind to the vessels wall and extravasate to the injured area in remarkably site-specific manner.

It has been discovered that CAR can bind to the normal blood vessels “activated” next to the site of the inflammatory insult (in addition to binding to the angiogenic blood vessels, that is, sprouting angiogenesis as shown earlier (U.S. Patent Application Publication No. 2009-0036349)). That process is shown in FIG. 1. The extravasation of both inflammatory cells and of CAR peptides is not a passive process; it is an active process driven by molecular cues in the normal blood vessels next to the site of the injury, which explains why it is site-specific and coordinated.

In some forms, the composition can selectively home to wounded tissue, regenerating tissue, sites of injury, surgical sites, sites of angiogenesis, sites of inflammation, sites of arthritis, lung tissue, pulmonary arterial hypertension lung vasculature, pulmonary arterial hypertension lesions, remodeled pulmonary arteries, or interstitial space of lungs.

In some forms, the composition can comprise a therapeutic agent, a therapeutic protein, a therapeutic compound, a therapeutic composition, an anti-inflammatory agent, an anti-arthritic agent, a TGF-β inhibitor, decorin, a systemic vasodilator, an anti-coagulant, tissue factor pathway inhibitor (TFPI), site-inactivated factor VIIa, a β-2 agonist, salmeterol, formoterol, N-Acetylcysteine (NAC), Superoxide Dismutase (SOD), a superoxide dismutase mimetic, EUK-8, an endothelin (ET-1) receptor antagonist, a prostacyclin derivative, a phosphodiesterase type 5 inhibitor, Ketoconazole, a toxin, a cytotoxic agent, an anti-angiogenic agent, a pro-angiogenic agent, an antibody, a small interfering RNA (siRNA), a microRNA (miRNA), a polypeptide, a nucleic acid molecule, a small molecule, a carrier, a vehicle, a virus, a phage, a viral particle, a phage particle, a viral capsid, a phage capsid, a virus-like particle, a liposome, a micelle, a bead, a nanoparticle, a microparticle, a detectable agent, a contrast agent, an imaging agent, a label, a labeling agent, a fluorophore, fluorescein, rhodamine, FAM, a radionuclide, indium-111, technetium-99, carbon-11, carbon-13, or a combination.

In some forms, the composition can comprise one or more accessory molecules. In some forms, the cell, tissue, or both can be exposed to a plurality of homing molecules. In some forms, the cell, tissue, or both can be exposed to a plurality of CAR compositions. Multiple different CAR peptides, CAR compounds, CAR conjugates, CAR compositions, or a combination can be used together. For example, a CARSKNKDC (SEQ ID NO:10) or CARSKNK peptide (SEQ ID NO:4) can be used together with one or multiple different CAR peptides, CAR compounds, CAR conjugates, CAR compositions, or a combination. In such combinations, the CARSKNKDC (SEQ ID NO: 10) or CARSKNK peptide (SEQ ID NO:4) itself can be combined in the same conjugate or composition with one or more accessory molecules, one or more homing molecules, etc.

The CAR peptide can be comprised in an amino acid sequence in a protein or peptide. In some forms, the protein or peptide can be internalized into a cell, penetrate tissue, or both when the amino acid sequence is present in the protein or peptide but not when the amino acid sequence is not present in the protein or peptide. In some forms, the protein or peptide can penetrate tissue when the amino acid sequence is present in the protein or peptide but not when the amino acid sequence is not present in the protein or peptide. In some forms, the protein or peptide can be internalized into a cell and penetrate tissue when the amino acid sequence is present in the protein or peptide but not when the amino acid sequence is not present in the protein or peptide.

The CAR peptide can be associated with one or more accessory molecules. For example, an accessory molecule can be a part of an amino acid sequence, a protein, or a peptide that comprises the CAR peptide. As another example, the accessory molecule can be covalently coupled or non-covalently associated with the CAR peptide or an amino acid sequence, a protein, or a peptide that comprises the CAR peptide. The accessory molecule can be separate from or overlapping with the CAR peptide. For example, some accessory molecules are amino acid sequences. This can allow the amino acid sequence consisting of the CAR peptide to overlap the amino acid sequence that consists of the accessory amino acid sequence. Alternatively the accessory peptide can be a separate entity that does not overlap with the CAR peptide. For example, a CREKA peptide, a NGR peptide, a iNGR, an RGD peptide, or a HER2 binding peptide that is not a CAR peptide can consist of amino acid sequence that does not overlap with a CAR peptide. In some forms, the accessory molecule can comprise a sequence in, for example, a CAR peptide that binds to a specific receptor distinct from the receptor for the CAR peptide. The amino acid sequence can comprise one or more accessory peptides. The protein or peptide can comprise one or more accessory peptides.

The peptide can be an activatable peptide. The activatable peptide can be a protease-activatable peptide. The CAR peptide can be an activatable CAR peptide. The activatable CAR peptide can be a protease-activatable CAR peptide. The CAR peptide can be at any position in the protein or peptide. The CAR peptide can be at other than at an end of the protein or peptide. The CAR peptide can be at the C-terminal end of the protein or peptide. The CAR peptide can be at the N-terminal end of the protein or peptide. The CAR conjugate can be an activatable CAR conjugate. The activatable CAR conjugate can be a protease-activatable CAR conjugate. The CAR conjugate can be any position in the protein or peptide. The CAR conjugate can be at other than at an end of the protein or peptide. The CAR conjugate can be at the C-terminal end of the protein or peptide. The CAR conjugate can be at the C-terminal end of the protein or peptide. The CAR composition can be an activatable CAR composition. The activatable CAR composition can be a protease-activatable CAR composition. The CAR composition can be any position in the protein or peptide. The CAR composition can be at other than at an end of the protein or peptide. The CAR composition can be at the C-terminal end of the protein or peptide. The CAR composition can be at the N-terminal end of the protein or peptide.

The CAR peptide can be associated with one or more homing molecules. For example, a homing molecule can be a part of an amino acid sequence, a protein, or a peptide that comprises the CAR peptide. As another example, the homing molecule can be covalently coupled or non-covalently associated with the CAR peptide or an amino acid sequence, a protein, or a peptide that comprises the CAR peptide. The homing molecule can be separate from or overlapping with the CAR peptide. For example, some homing molecules are amino acid sequences. This can allow the amino acid sequence consisting of the CAR peptide to overlap the amino acid sequence that consists of the homing amino acid sequence. Alternatively the homing peptide can be a separate entity that does not overlap with the CAR peptide. For example, a CREKA peptide, a NGR peptide, a iNGR, an RGD peptide, or a HER2 binding peptide that is not a CAR peptide can consist of amino acid sequence that does not overlap with a CAR peptide. In some forms, the homing molecule can comprise a sequence in, for example, a peptide that binds to a specific receptor distinct from the receptor for the CAR peptide.

Many homing molecules and homing peptides home to the vasculature of the target tissue. However, for the sake of convenience homing is referred to in some places herein as homing to the tissue associated with the vasculature to which the homing molecule or homing peptide may actually home. Thus, for example, a homing peptide that homes to lung vasculature can be referred to herein as homing to lung tissue or to lung cells. By including or associating a homing molecule or homing peptide with, for example, a protein, peptide, or amino acid sequence, the protein, peptide, or amino acid sequence can be targeted or can home to the target of the homing molecule or homing peptide. In this way, the protein, peptide, or amino acid sequence can be said to home to the target of the homing molecule or homing peptide. For convenience and unless otherwise indicated, reference to homing of a protein, peptide, amino acid sequence, etc. is intended to indicate that the protein, peptide, amino acid sequence, etc. includes or is associated with an appropriate homing molecule or homing peptide.

The amino acid sequence can be selected for internalization into a cell. The amino acid sequence can be selected for tissue penetration. The amino acid sequence can be selected for internalization into a cell and tissue penetration. The amino acid sequence can comprise one or more homing peptides. The amino acid sequence can comprise a tCAR peptide.

The CAR peptide can be the only functional internalization element in the CAR composition, conjugate, molecule, protein, peptide, etc., the CAR peptide can be the only functional tissue penetration element in the CAR composition, conjugate, molecule, protein, peptide, etc., or both. The selected amino acid sequence can be the only functional internalization element in the CAR composition, conjugate, molecule, protein, peptide, etc., the selected amino acid sequence can be the only functional tissue penetration element in the CAR composition, conjugate, molecule, protein, peptide, etc., or both.

Disclosed herein is a method of forming a CAR composition, the method comprising selecting an amino acid sequence for internalization into a cell, and causing a CAR peptide to be covalently coupled to or non-covalently associated with the selected amino acid sequence, wherein the CAR composition comprises the selected amino acid sequence and the coupled or associated CAR peptide.

Disclosed is a method of making a CAR composition comprising: (a) selecting an amino acid sequence for internalization into a cell, (b) causing a CAR peptide to be covalently coupled to or non-covalently associated with the selected amino acid sequence, wherein the CAR composition comprises the selected amino acid sequence and the coupled or associated CAR peptide.

Further disclosed is a method of delivering a composition into a cell, the method comprising: exposing the cell to the composition and a CAR composition comprising an activatable CAR peptide, whereupon a cleaving agent activates the activatable CAR peptide of the CAR composition, wherein the CAR composition can then enter the cell, thereby delivering the composition into the cell.

Further disclosed is a method of causing a composition to penetrate tissue, the method comprising: exposing the tissue to the composition and a CAR composition comprising an activatable CAR peptide, whereupon a cleaving agent activates the activatable CAR peptide of the CAR composition, wherein the CAR composition can then enter and pass cells in the tissue, thereby causing the composition to penetrate the tissue.

Cells that can internalize a CAR peptide can be identified by (a) exposing a cell to a CAR peptide; and (b) determining if the CAR peptide was internalized. The cell can be in an assay, for example. Cells that can internalize an activatable peptide can be identified by (a) exposing a cell to an activatable peptide; (b) determining if the activatable peptide was internalized. The activatable peptide can be unblocked before exposure to the cell, but does not need to be. This can be used to test the blocking ability of the blocker, for example.

As used herein, reference to components (such as a CAR peptide and an accessory molecule) as being “not covalently coupled” means that the components are not connected via covalent bonds (for example, that the CAR peptide and the accessory molecule are not connected via covalent bonds). That is, there is no continuous chain of covalent bonds between, for example, the CAR peptide and the accessory molecule. Conversely, reference to components (such as a CAR peptide and an accessory molecule) as being “covalently coupled” means that the components are connected via covalent bonds (for example, that the CAR peptide and the accessory molecule are connected via covalent bonds). That is, there is a continuous chain of covalent bonds between, for example, the CAR peptide and the accessory molecule. Components can be covalently coupled either directly or indirectly. Direct covalent coupling refers to the presence of a covalent bond between atoms of each of the components. Indirect covalent coupling refers to the absence of a covalent bond between atoms of each of the components. That is, some other atom or atoms not belonging to either of the coupled components intervenes between atoms of the components. Both direct and indirect covalent coupling involve a continuous chain of covalent bonds.

Non-covalent association refers to association of components via non-covalent bonds and interactions. A non-covalent association can be either direct or indirect. A direct non-covalent association refers to a non-covalent bond involving atoms that are each respectively connected via a chain of covalent bonds to the components. Thus, in a direct non-covalent association, there is no other molecule intervening between the associated components. An indirect non-covalent association refers to any chain of molecules and bonds linking the components where the components are not covalently coupled (that is, there is a least one separate molecule other than the components intervening between the components via non-covalent bonds).

Reference to components (such as a CAR peptide and an accessory molecule) as not being “non-covalently associated” means that there is no direct or indirect non-covalent association between the components. That is, for example, no atom covalently coupled to a CAR peptide is involved in a non-covalent bond with an atom covalently coupled to an accessory molecule. Within this meaning, a CAR peptide and an accessory molecule can be together in a composition where they are indirectly associated via multiple intervening non-covalent bonds while not being non-covalently associated as that term is defined herein. For example, a CAR peptide and an accessory molecule can be mixed together in a carrier where they are not directly non-covalently associated. A CAR peptide and an accessory molecule that are referred to as not indirectly non-covalently associated cannot be mixed together in a continuous composition. Reference to components (such as a CAR peptide and an accessory molecule) as not being “directly non-covalently associated” means that there is no direct non-covalent association between the components (an indirect non-covalent association may be present). Reference to components (such as a CAR peptide and an accessory molecule) as not being “indirectly non-covalently associated” means that there is no direct or indirect non-covalent association between the components.

It is understood that components can be non-covalently associated via multiple chains and paths including both direct and indirect non-covalent associations. For the purposes of these definitions, the presence a single direct non-covalent association makes the association a direct non-covalent association even if there are also indirect non-covalent associations present. Similarly, the presence of a covalent connection between components means the components are covalently coupled even if there are also non-covalent associations present. It is also understood that covalently coupled components that happened to lack any non-covalent association with each other are not considered to fall under the definition of components that are not non-covalently associated.

The CAR peptide can be associated with one or more accessory molecules. For example, an accessory molecule can be a part of an amino acid sequence, protein, peptide, conjugate, or composition that comprises the CAR peptide. As another example, the accessory molecule can be covalently coupled or non-covalently associated with the CAR peptide or an amino acid sequence, protein, peptide, conjugate, or composition that comprises the CAR peptide. Accessory molecules can be any molecule, compound, component, etc. that has a useful function and that can be used in combination with a CAR composition, CAR conjugate, CAR molecule, CAR protein, and/or CAR peptide. Examples of useful accessory molecules include homing molecules, targeting molecules, affinity ligands, cell penetrating molecules, endosomal escape molecules, subcellular targeting molecules, nuclear targeting molecules. Different accessory molecules can have similar or different functions from each other. Accessory molecules having similar functions, different functions, or both, can be associated a CAR composition, CAR conjugate, CAR molecule, CAR protein, and/or CAR peptide.

The accessory molecule can be separate from or overlapping with the CAR peptide. For example, some accessory molecules are amino acid sequences. This can allow the amino acid sequence consisting of the CAR peptide to overlap the amino acid sequence that consists of the accessory amino acid sequence. For example, iRGD, CAR, LyP-1, iNGR, and RGR peptides each contain both an accessory sequence and CendR sequence overlapping with one another in the peptide. The accessory molecule can be a separate entity that does not overlap with the CAR peptide. For example, a CREKA peptide, a NGR peptide, a iNGR, an RGD peptide, or a HER2 binding peptide that is not a CAR peptide can consist of amino acid sequence that does not overlap with a CAR peptide. In some forms, the accessory molecule can comprise a sequence in, for example, a peptide that binds to a specific receptor distinct from the receptor for the CAR peptide.

The CAR peptide can be associated with one or more accessory molecules. For example, an accessory molecule can be a part of an amino acid sequence, protein, peptide, conjugate, or composition that comprises the CAR peptide. As another example, the accessory molecule can be covalently coupled or non-covalently associated with the CAR peptide or an amino acid sequence, protein, peptide, conjugate, or composition that comprises the CAR peptide. The CAR conjugate can be associated with one or more accessory molecules. For example, an accessory molecule can be a part of a conjugate or composition that comprises the CAR conjugate. As another example, the accessory molecule can be covalently coupled or non-covalently associated with the CAR conjugate or a conjugate or composition that comprises the CAR conjugate. The CAR composition can be associated with one or more accessory molecules. For example, an accessory molecule can be a part of a composition that comprises the CAR composition. As another example, the accessory molecule can be covalently coupled or non-covalently associated with the CAR composition or a composition that comprises the CAR composition.

The amino acid sequence can be associated with one or more accessory molecules. For example, an accessory molecule can be a part of an amino acid sequence, protein, peptide, conjugate, or composition that comprises the amino acid sequence. As another example, the accessory molecule can be covalently coupled or non-covalently associated with the amino acid sequence or an amino acid sequence, protein, peptide, conjugate, or composition that comprises the amino acid sequence. For example, the amino acid sequences can comprise a iRGD peptide, a CAR peptide, a LyP-1 peptide, a RGR peptide, a CREKA peptide, a NGR peptide, iNGR, a HER2 binding peptide, or a RGD peptide that is not a CAR peptide, or a combination. The amino acid sequence can comprise a CREKA peptide. The protein or peptide can be associated with one or more accessory molecules. For example, an accessory molecule can be a part of a protein, peptide, conjugate, or composition that comprises the peptide. As another example, the accessory molecule can be covalently coupled or non-covalently associated with the peptide or a protein, peptide, conjugate, or composition that comprises the peptide. For example, an accessory molecule can be a part of a protein, conjugate, or composition that comprises the protein. As another example, the accessory molecule can be covalently coupled or non-covalently associated with the protein or a protein, conjugate, or composition that comprises the protein. For example, the protein or peptide can comprise a iRGD peptide, a CAR peptide, a LyP-1 peptide, a RGR peptide, a CREKA peptide, a NGR peptide, iNGR, a HER2 binding peptide, or a RGD peptide that is not a CAR peptide, or a combination. The conjugate can be associated with one or more accessory molecules. For example, an accessory molecule can be a part of a conjugate or composition that comprises the conjugate. As another example, the accessory molecule can be covalently coupled or non-covalently associated with the conjugate or a conjugate or composition that comprises the conjugate. For example, the conjugate can comprise a iRGD peptide, a CAR peptide, a LyP-1 peptide, a RGR peptide, a CREKA peptide, a NGR peptide, iNGR, a HER2 binding peptide, or a RGD peptide that is not a CAR peptide, or a combination. The composition can be associated with one or more accessory molecules. For example, an accessory molecule can be a part of a composition that comprises the composition. As another example, the accessory molecule can be covalently coupled or non-covalently associated with the composition or a composition that comprises the composition. For example, the composition can comprise a iRGD peptide, a CAR peptide, a LyP-1 peptide, a RGR peptide, a CREKA peptide, a NGR peptide, iNGR, a HER2 binding peptide, or a RGD peptide that is not a CAR peptide, or a combination.

The CAR peptide can be associated with one or more homing molecules. For example, a homing molecule can be a part of an amino acid sequence, protein, peptide, conjugate, or composition that comprises the CAR peptide. As another example, the homing molecule can be covalently coupled or non-covalently associated with the CAR peptide or an amino acid sequence, protein, peptide, conjugate, or composition that comprises the CAR peptide. The homing molecule can be separate from or overlapping with the CAR peptide. For example, some homing molecules are amino acid sequences. This can allow the amino acid sequence consisting of the CAR peptide to overlap the amino acid sequence that consists of the homing amino acid sequence. For example, iRGD, CAR, LyP-1, iNGR, and RGR peptides each contain both a homing sequence and CendR sequence overlapping with one another in the peptide. The homing molecule can be a separate entity that does not overlap with the CAR peptide. For example, a CREKA peptide, a NGR peptide, a iNGR, an RGD peptide, or a HER2 binding peptide that is not a CAR peptide can consist of amino acid sequence that does not overlap with a CAR peptide. In some forms, the homing molecule can comprise a sequence in, for example, a CAR peptide that binds to a specific receptor distinct from the receptor for the CAR peptide.

The CAR peptide can be associated with one or more homing molecules. For example, a homing molecule can be a part of an amino acid sequence, protein, peptide, conjugate, or composition that comprises the CAR peptide. As another example, the homing molecule can be covalently coupled or non-covalently associated with the CAR peptide or an amino acid sequence, protein, peptide, conjugate, or composition that comprises the CAR peptide. The CAR conjugate can be associated with one or more homing molecules. For example, a homing molecule can be a part of a conjugate or composition that comprises the CAR conjugate. As another example, the homing molecule can be covalently coupled or non-covalently associated with the CAR conjugate or a conjugate or composition that comprises the CAR conjugate. The CAR composition can be associated with one or more homing molecules. For example, a homing molecule can be a part of a composition that comprises the CAR composition. As another example, the homing molecule can be covalently coupled or non-covalently associated with the CAR composition or a composition that comprises the CAR composition.

The amino acid sequence can be associated with one or more homing molecules. For example, a homing molecule can be a part of an amino acid sequence, protein, peptide, conjugate, or composition that comprises the amino acid sequence. As another example, the homing molecule can be covalently coupled or non-covalently associated with the amino acid sequence or an amino acid sequence, protein, peptide, conjugate, or composition that comprises the amino acid sequence. For example, the amino acid sequences can comprise a iRGD peptide, a CAR peptide, a LyP-1 peptide, a RGR peptide, a CREKA peptide, a NGR peptide, iNGR, a HER2 binding peptide, or a RGD peptide that is not a CAR peptide, or a combination. The amino acid sequence can comprise a CREKA peptide. The protein or peptide can be associated with one or more homing molecules. For example, a homing molecule can be a part of a protein, peptide, conjugate, or composition that comprises the peptide. As another example, the homing molecule can be covalently coupled or non-covalently associated with the peptide or a protein, peptide, conjugate, or composition that comprises the peptide. For example, a homing molecule can be a part of a protein, conjugate, or composition that comprises the protein. As another example, the homing molecule can be covalently coupled or non-covalently associated with the protein or a protein, conjugate, or composition that comprises the protein. For example, the protein or peptide can comprise a iRGD peptide, a CAR peptide, a LyP-1 peptide, a RGR peptide, a CREKA peptide, a NGR peptide, iNGR, a HER2 binding peptide, or a RGD peptide that is not a CAR peptide, or a combination. The protein or peptide can comprise iRGD. The protein or peptide can comprise a CAR peptide, a LyP-1 peptide. The protein or peptide can comprise iNGR. The protein or peptide can comprise RGR peptide. The protein or peptide can comprise a CREKA peptide. The conjugate can be associated with one or more homing molecules. For example, a homing molecule can be a part of a conjugate or composition that comprises the conjugate. As another example, the homing molecule can be covalently coupled or non-covalently associated with the conjugate or a conjugate or composition that comprises the conjugate. For example, the conjugate can comprise a iRGD peptide, a CAR peptide, a LyP-1 peptide, a RGR peptide, a CREKA peptide, a NGR peptide, iNGR, a HER2 binding peptide, or a RGD peptide that is not a CAR peptide, or a combination. The conjugate can comprise iRGD. The conjugate can comprise a CAR peptide, a LyP-1 peptide. The conjugate can comprise iNGR. The conjugate can comprise RGR peptide. The conjugate can comprise a CREKA peptide. The composition can be associated with one or more homing molecules. For example, a homing molecule can be a part of a composition that comprises the composition. As another example, the homing molecule can be covalently coupled or non-covalently associated with the composition or a composition that comprises the composition. For example, the composition can comprise a iRGD peptide, a CAR peptide, a LyP-1 peptide, a RGR peptide, a CREKA peptide, a NGR peptide, iNGR, a HER2 binding peptide, or a RGD peptide that is not a CAR peptide, or a combination. The composition can comprise iRGD. The composition can comprise a CAR peptide. The composition can comprise a LyP-1 peptide. The composition can comprise iNGR. The composition can comprise RGR peptide. The composition can comprise a CREKA peptide.

The amino acid sequence can be selected for internalization into a cell. The amino acid sequence can be selected for tissue penetration. The amino acid sequence can be selected for internalization into a cell and tissue penetration. The protein or peptide can be selected for internalization into a cell. The protein or peptide can be selected for tissue penetration. The protein or peptide can be selected for internalization into a cell and tissue penetration. The conjugate can be selected for internalization into a cell. The conjugate can be selected for tissue penetration. The conjugate can be selected for internalization into a cell and tissue penetration. The composition can be selected for internalization into a cell. The composition can be selected for tissue penetration. The composition can be selected for internalization into a cell and tissue penetration.

The CAR peptide, CAR conjugate, CAR composition, amino acid sequence, protein or peptide, conjugate, composition, or a combination can selectively home to regenerating tissue, sites of injury, surgical sites, sites of angiogenesis, sites of inflammation, sites of arthritis, lung tissue, pulmonary arterial hypertension lung vasculature, pulmonary arterial hypertension lesions, remodeled pulmonary arteries, or interstitial space of lungs.

The CAR peptide, CAR conjugate, CAR composition, amino acid sequence, protein or peptide, conjugate, composition, or a combination can selectively home to lung tissue. The CAR peptide, CAR conjugate, CAR composition, amino acid sequence, protein or peptide, conjugate, composition, or a combination can selectively home to lung vasculature. The CAR peptide, CAR conjugate, CAR composition, amino acid sequence, protein or peptide, conjugate, composition, or a combination can selectively home to heart tissue. The CAR peptide, CAR conjugate, CAR composition, amino acid sequence, protein or peptide, conjugate, composition, or a combination can selectively home to heart vasculature. The CAR peptide, CAR conjugate, CAR composition, amino acid sequence, protein or peptide, conjugate, composition, or a combination can selectively home to brain cells, brain stem cells, brain tissue, and/or brain vasculature, kidney cells, kidney stem cells, kidney tissue, and/or kidney vasculature, skin cells, skin stem cells, skin tissue, and/or skin vasculature, lung cells, lung tissue, and/or lung vasculature, pancreatic cells, pancreatic tissue, and/or pancreatic vasculature, intestinal cells, intestinal tissue, and/or intestinal vasculature, adrenal gland cells, adrenal tissue, and/or adrenal vasculature, retinal cells, retinal tissue, and/or retinal vasculature, liver cells, liver tissue, and/or liver vasculature, prostate cells, prostate tissue, and/or prostate vasculature, endometriosis cells, endometriosis tissue, and/or endometriosis vasculature, ovary cells, ovary tissue, and/or ovary vasculature, bone cells, bone tissue, and/or bone vasculature, bone marrow cells, bone marrow tissue, and/or bone marrow vasculature, cartilage cells, cartilage tissue, and/or cartilage vasculature, stem cells, embryonic stem cells, pluripotent stem cells, induced pluripotent stem cells, adult stem cells, hematopoietic stem cells, neural stem cells, mesenchymal stem cells, mammary stem cells, endothelial stem cells, olfactory adult stem cells, neural crest stem cells, cancer stem cells, blood cells, erythrocytes, platelets, leukocytes, granulocytes, neutrophils, eosinphils, basophils, lymphoid cells, lymphocytes, monocytes, wound vasculature, vasculature of injured tissue, vasculature of inflamed tissue, atherosclerotic plaques, or a combination.

CAR compositions, CAR conjugates, CAR molecules, CAR proteins, and CAR peptides can be designed and produced in any suitable manner. For example, the tCAR peptide in the disclosed CAR compositions, CAR conjugates, CAR molecules, and CAR proteins can be designed or produced by selecting an amino acid sequence for internalization into a cell and/or penetration of tissue, wherein a protein or peptide comprises the selected amino acid sequence. As another example, the tCAR peptide in the disclosed CAR compositions, CAR conjugates, CAR molecules, and CAR proteins can be designed or produced by selecting an amino acid sequence for internalization into a cell and/or penetration of tissue, wherein the amino acid sequence comprises a C-terminal element, wherein a protein or peptide comprises the selected amino acid sequence, wherein the selected amino acid sequence is at the C-terminal end of the protein or peptide.

The peptide can be an activatable peptide. The activatable peptide can be a protease-activatable peptide. The protease-activatable peptide can be activatable by a serine protease, plasmin, a plasminogen activator, urokinase, a proprotein convertase, a furin, a carboxypeptidase, carboxypeptidase A, a glutamate-specific carboxypeptidase, a proline-specific carboxypeptidase, PSMA, or a combination.

The CAR peptide can be comprised in an amino acid sequence. The amino acid sequence can be comprised in a protein or peptide. The CAR peptide can be comprised in a protein or peptide. In some forms, the protein or peptide can be internalized into a cell, penetrate tissue, or both when the amino acid sequence is present in the protein or peptide but not when the amino acid sequence is not present in the protein or peptide. In some forms, the protein or peptide can penetrate tissue when the amino acid sequence is present in the protein or peptide but not when the amino acid sequence is not present in the protein or peptide. In some forms, the protein or peptide can be internalized into a cell and penetrate tissue when the amino acid sequence is present in the protein or peptide but not when the amino acid sequence is not present in the protein or peptide.

The amino acid sequence can be associated with one or more accessory molecules. The protein or peptide can be associated with one or more accessory molecules. One or more of the accessory molecules can be independently a homing molecule, a targeting molecule, an affinity ligand, a cell penetrating peptide, an endosomal escape molecule, a subcellular targeting molecule, a nuclear targeting molecule, or a combination. One or more of the accessory molecules can be homing molecules. One or more of the homing molecules can be independently an RGD peptide, iRGD, a CAR peptide, a LyP-1 peptide, NGR peptide, iNGR, RGR peptide, HER2 binding peptide, or a combination.

The amino acid sequence can be selected for internalization into a cell. The amino acid sequence can be selected for tissue penetration. The amino acid sequence can be selected for internalization into a cell and tissue penetration.

The CAR peptide can be comprised in a CAR composition. The CAR composition can comprise one or more accessory molecules. The CAR composition can comprise one or more homing molecules. The CAR peptide can be comprised in a CAR conjugate. The CAR conjugate can comprise one or more accessory molecules. The CAR conjugate can comprise one or more homing molecules. The cell, tissue, or both can be exposed to a plurality of accessory molecules. The cell, tissue, or both can be exposed to a plurality of homing molecules. The cell, tissue, or both can be exposed to a plurality of CAR peptides.

As used herein, “selecting an amino acid sequence for internalization into a cell” refers to selecting, identifying designing or otherwise categorizing an amino acid sequence with the specific intention of obtaining entry into a cell of a protein or peptide that is comprised of the amino acid sequence. Thus, for example, selecting an amino acid sequence for some purpose or capability other than obtaining entry into a cell of a protein or peptide that is comprised of the amino acid sequence and in the absence of an intention of obtaining entry into a cell of a protein or peptide that is comprised of the amino acid sequence does not constitute “selecting an amino acid sequence for internalization into a cell.” Selecting an amino acid sequence for some purpose or capability as well as for obtaining entry into a cell of a protein or peptide that is comprised of the amino acid sequence does constitute “selecting an amino acid sequence for internalization into a cell.” Thus, the presence of additional goals or purposes does not alter that selection of an amino acid sequence at least with the specific intention of obtaining entry into a cell of a protein or peptide that is comprised of the amino acid sequence constitutes “selecting an amino acid sequence for internalization into a cell.”

As used herein, “selecting an amino acid sequence for penetration of tissue” refers to selecting, identifying designing or otherwise categorizing an amino acid sequence with the specific intention of obtaining entry into tissue (that is, tissue penetration) of a protein or peptide that is comprised of the amino acid sequence. Thus, for example, selecting an amino acid sequence for some purpose or capability other than obtaining entry into tissue of a protein or peptide that is comprised of the amino acid sequence and in the absence of an intention of obtaining entry into tissue of a protein or peptide that is comprised of the amino acid sequence does not constitute “selecting an amino acid sequence for penetration of tissue.” Selecting an amino acid sequence for some purpose or capability as well as for obtaining entry into tissue of a protein or peptide that is comprised of the amino acid sequence does constitute “selecting an amino acid sequence for penetration of tissue.” Thus, the presence of additional goals or purposes does not alter that selection of an amino acid sequence at least with the specific intention of obtaining entry into tissue of a protein or peptide that is comprised of the amino acid sequence constitutes “selecting an amino acid sequence for penetration of tissue.”

As used herein, “selecting an amino acid sequence for internalization into a cell and/or penetration of tissue” refers to selecting, identifying designing or otherwise categorizing an amino acid sequence with the specific intention of obtaining entry into either or both a cell and tissue of a protein or peptide that is comprised of the amino acid sequence. Thus, for example, selecting an amino acid sequence for some purpose or capability other than obtaining entry into a cell, tissue, or both of a protein or peptide that is comprised of the amino acid sequence and in the absence of an intention of obtaining entry into a cell, tissue, or both of a protein or peptide that is comprised of the amino acid sequence does not constitute “selecting an amino acid sequence for internalization into a cell and/or penetration of tissue.” Selecting an amino acid sequence for some purpose or capability as well as for obtaining entry into either or both a cell and tissue of a protein or peptide that is comprised of the amino acid sequence does constitute “selecting an amino acid sequence for internalization into a cell and/or penetration of tissue.” Thus, the presence of additional goals or purposes does not alter that selection of an amino acid sequence at least with the specific intention of obtaining entry into a cell, tissue, or both of a protein or peptide that is comprised of the amino acid sequence constitutes “selecting an amino acid sequence for internalization into a cell and/or penetration of tissue.”

As used herein, unless the context indicates otherwise, “selecting a composition for internalization into a cell” refers to selecting, identifying designing or otherwise categorizing a composition and a CAR composition, CAR conjugate, CAR molecule, CAR protein, or CAR peptide with the specific intention of obtaining entry into a cell of both the composition and the CAR composition, CAR conjugate, CAR molecule, CAR protein, or CAR peptide. Thus, for example, selecting a composition for some purpose or capability other than obtaining entry into a cell in combination with entry of a selected CAR composition, CAR conjugate, CAR molecule, CAR protein, or CAR peptide and in the absence of an intention of obtaining entry into a cell of both the composition and the CAR composition, CAR conjugate, CAR molecule, CAR protein, or CAR peptide does not constitute “selecting composition for internalization into a cell.” Selecting a composition for some purpose or capability as well as for obtaining entry into a cell of the composition does constitute “selecting composition for internalization into a cell.” Thus, the presence of additional goals or purposes does not alter that selection of a composition at least with the specific intention of obtaining entry into a cell of a composition constitutes “selecting a composition for internalization into a cell.”

As used herein, unless the context indicates otherwise, “selecting a composition for penetration of tissue” refers to selecting, identifying designing or otherwise categorizing a composition and a CAR composition, CAR conjugate, CAR molecule, CAR protein, or CAR peptide with the specific intention of obtaining entry into tissue (that is, tissue penetration) of both the composition and the CAR composition, CAR conjugate, CAR molecule, CAR protein, or CAR peptide. Thus, for example, selecting a composition for some purpose or capability other than obtaining entry into tissue in combination with entry of a selected CAR composition, CAR conjugate, CAR molecule, CAR protein, or CAR peptide and in the absence of an intention of obtaining entry into tissue of both the composition and the CAR composition, CAR conjugate, CAR molecule, CAR protein, or CAR peptide does not constitute “selecting composition for penetration of tissue.” Selecting a composition for some purpose or capability as well as for obtaining entry into tissue of the composition does constitute “selecting composition for penetration of tissue.” Thus, the presence of additional goals or purposes does not alter that selection of a composition at least with the specific intention of obtaining entry into tissue of a composition constitutes “selecting a composition for penetration of tissue.”

As used herein, unless the context indicates otherwise, “selecting a composition for internalization into a cell and/or penetration of tissue” refers to selecting, identifying designing or otherwise categorizing a composition and a CAR composition, CAR conjugate, CAR molecule, CAR protein, or CAR peptide with the specific intention of obtaining entry into either or both a cell and tissue of both the composition and the CAR composition, CAR conjugate, CAR molecule, CAR protein, or CAR peptide. Thus, for example, selecting a composition for some purpose or capability other than obtaining entry into either or both a cell and tissue in combination with entry of a selected CAR composition, CAR conjugate, CAR molecule, CAR protein, or CAR peptide and in the absence of an intention of obtaining entry into either or both a cell and tissue of both the composition and the CAR composition, CAR conjugate, CAR molecule, CAR protein, or CAR peptide does not constitute “selecting composition for internalization into a cell and/or penetration of tissue.” Selecting a composition for some purpose or capability as well as for obtaining entry into either or both a cell and tissue of the composition does constitute “selecting composition for internalization into a cell and/or penetration of tissue.” Thus, the presence of additional goals or purposes does not alter that selection of a composition at least with the specific intention of obtaining entry into either or both a cell and tissue of a composition constitutes “selecting a composition for internalization into a cell and/or penetration of tissue.”

As used herein, “causing a compound or composition to be covalently coupled or non-covalently associated” with something else refers to any action that results in a compound or composition that is not covalently coupled or non-covalently associated with the something else becoming or coming into the state of being covalently coupled or non-covalently associated with the something else. As an example, covalently coupling a homing molecule to a CAR peptide constitutes “causing a homing molecule to be covalently coupled or non-covalently associated” with the CAR peptide. As another example, a CAR peptide that starts as a nonexistent concept and then is synthesized as part of a composition that includes the thing to which the CAR peptide is to be coupled or associated constitutes “causing a CAR peptide to be covalently coupled or non-covalently associated” with the thing. For example, synthesis of a peptide that includes both an amino acid sequence of interest and an amino acid sequence comprising a C-terminal element constitutes causing the amino acid sequence of interest to be covalently coupled or non-covalently associated with the amino acid sequence comprising a C-terminal element. However, and in general, synthesis of a protein or peptide that naturally includes both the amino acid sequence of interest and an amino acid sequence comprising a C-terminal element can be excluded as a process of “causing the amino acid sequence of interest to be covalently coupled or non-covalently associated” with the amino acid sequence comprising a C-terminal element.

As used herein, “causing a composition to be covalently coupled or non-covalently associated” with something else refers to any action that results in a composition that is not and the something else becoming or coming into the state of being and the something else. More clearly, “causing a composition to be covalently coupled or non-covalently associated” with something else refers to any action that results in a composition and the something else becoming or coming into the state of being covalently coupled or non-covalently associated. As an example, covalently coupling a composition to another composition constitutes “causing a composition to be covalently coupled or non-covalently associated” with the other composition. As another example, a composition that starts as a nonexistent concept and then is synthesized as part of a composition that includes the thing to which the composition is to be coupled or associated constitutes “causing a composition to be covalently coupled or non-covalently associated” with the thing.

CAR peptides can be composed of, for example, amino acids, amino acid analogs, peptide analogs, amino acid mimetics, peptide mimetics, etc. Although structures, design, etc. of CAR peptides is described herein in terms of amino acids and peptides composed of amino acids for convenience, it is understood that analogous analogs, mimetics, modified forms, etc. of amino acids and peptides can also be used as CAR peptides and designed using similar principles.

Any component, such as the components disclosed herein, can overlap, be adjacent to, and/or be upstream, downstream, or both of a peptide, such as a CAR peptide. Examples of such components include accessory molecules, homing molecules, protease cleavage sites, etc. It is useful to have some components coupled to or associated with a peptide, such as a CAR peptide to be downstream (C-terminal) of the peptide. For example, activatable peptide having an accessory protein or a homing peptide downstream of the peptide (and thus downstream from the cleavage site for activation) will be separated from the peptide when it is activated. As another example, activatable peptides having an accessory molecule or a homing molecule downstream of the peptide (and thus downstream from the cleavage site for activation) will be separated from the peptide when it is activated. This can have some advantages such as making the peptide function more efficient or reducing the chance for extraneous effects of the eliminated component.

As used herein, “activatable peptide” refers to a peptide, such as a CAR peptide, having a molecule, moiety, nanoparticle, compound or other composition covalently coupled to the peptide, such as to the terminal carboxyl group of the peptide, where the molecule, moiety, nanoparticle, compound or other composition can block internalization and/or tissue penetration of the CAR composition, conjugate, molecule, protein, peptide, etc. and where the molecule, moiety, nanoparticle, compound or other composition can be removed (to expose the terminal carboxy group of the peptide, for example). For example, the activatable peptide can be on the C-terminal end of the protein or peptide, and can prevent the peptide from being internalized and/or from penetrating tissue. As another example, the activatable peptide can be on the N-terminal end of the protein or peptide, and can prevent the peptide from being internalized and/or from penetrating tissue. As another example, the activatable peptide can be other than on the N-terminal end or C-terminal end of the protein or peptide, and can prevent the peptide from being internalized and/or from penetrating tissue. The molecule, nanoparticle, moiety, compound or other composition covalently coupled to the peptide can be referred to as the “blocking group.” For example, the blocking group can be coupled to the terminal carboxyl group of the C-terminal arginine of the CAR peptide, to the C-terminal amino acid of the CAR peptide, or to an amino acid of the CAR peptide other than the C-terminal amino acid. As another example, the blocking group can be coupled to the CAR sequence wherever it appears in the protein or peptide. The blocking group can also be coupled, or associated with a part of a CAR composition, conjugate, molecule, protein, peptide, etc. other than the CAR peptide so long as it can prevent the CAR peptide from being internalized and/or from penetrating tissue. For example, the blocking group can be coupled to the terminal carboxy group, the terminal amino group, the C-terminal amino acid, the N-terminal amino acid, an amino acid in the CAR or tCAR amino acid sequence, or elsewhere in the peptide. A CAR composition comprising an activatable peptide, such as an activatable CAR peptide, can be referred to as an activatable CAR composition. A CAR molecule comprising an activatable peptide, such as an activatable CAR peptide, can be referred to as an activatable CAR molecule. A CAR conjugate comprising an activatable peptide, such as an activatable CAR peptide, can be referred to as an activatable CAR conjugate. A CAR protein comprising an activatable peptide, such as an activatable CAR peptide, can be referred to as an activatable CAR protein. A CAR peptide comprising an activatable peptide can be referred to as an activatable CAR peptide. The blocking group can comprise or consist of an amino acid or an amino acid sequence.

An activatable peptide, such as an activatable CAR peptide, can be blocked from internalization into a cell, from tissue penetration, or both. Generally, an activatable peptide, such as an activatable CAR peptide, will be blocked from both internalization into a cell and penetration of tissue. Such activatable peptides can be referred to as activatable internalization and penetrating peptides. However, some activatable peptides could be blocked only from tissue penetration or only from internalization into a cell. Such activatable peptides can be referred to as activatable internalization peptides (for peptides that are blocked only from internalization into a cell) or as activatable penetrating peptides (for peptides that are blocked only from penetration of tissue). Generally, internalization peptides that are activatable will be activatable internalization peptides. Similarly, penetrating peptides that are activatable generally will be activatable penetrating peptides. Internalization and penetrating peptides that are activatable will be activatable internalization and penetrating peptides. Removal of the blocking group will allow the peptide to be internalized into a cell, penetrate tissue, or both.

The cleavable bond of an activatable peptide, such as an activatable CAR peptide, can be cleaved in any suitable way. For example, the cleavable bond can be cleaved enzymatically or non-enzymatically. For enzymatic cleavage, the cleaving enzyme can be supplied or can be present at a site where the peptide is delivered, homes, travels or accumulates. For example, the enzyme can be present in proximity to a cell to which the peptide is delivered, homes, travels, or accumulates. For non-enzymatic cleavage, the peptide can be brought into contact with a cleaving agent, can be placed in cleaving conditions, or both. A cleaving agent is any substance that can mediate or stimulate cleavage of the cleavable bond. A non-enzymatic cleaving agent is any cleaving agent except enzymes. Cleaving conditions can be any solution or environmental conditions that can mediate or stimulate cleavage of the cleavable bond. For example, some labile bonds can be cleaved in acid conditions, alkaline conditions, in the presence of a reactive group, etc. Non-enzymatic cleaving conditions are any cleaving conditions except the presence of enzymes. Non-agent cleaving conditions are any cleaving conditions except the presence of cleaving agents.

Activatable peptides, such as activatable CAR peptides, can be activatable in broad or narrow circumstances. Generally, activatable peptides are activatable relative to a specific agent or group of agents that can activate the peptides. Thus, for example, a particular activatable peptide may only be activatable by certain proteases. Such a peptide can be referred to as an activatable peptide but can also be referred to as being activatable by the particular proteases.

A “protease-activatable peptide” (or “protease-activated peptide”) refers to an activatable peptide where the blocking group is coupled to the peptide via a peptide bond and where the peptide bond can be cleaved by a protease. Cleavage of this peptide bond in a protease-activatable peptide makes the peptide capable of internalization into a cell and/or of tissue penetration. In one example, the blocking group can be coupled to the internalizing peptide via a cleavable or labile bond. The cleavable bond can be cleaved by, for example, an enzyme or a chemical compound. Cleavage or ‘labilization’ of the bond in an activatable peptide makes the peptide capable of internalization into a cell and/or of tissue penetration. Such cleavage or ‘labilization’ can be referred to as activation of the peptide. A protease-activatable peptide is a form of activatable peptide. The amino acids of an activatable CAR peptide, can be selected for specific purposes. For example, the amino acids preceding the beginning or following the end of the CAR sequence can be chosen to form all or a portion of a protease recognition sequence. This would be useful, for example, to specify or enable cleavage of a peptide having the CAR peptide that is activated by cleavage following the end of the CAR sequence. Examples of such amino acid choices are shown in Tables 1 and 2 of U.S. Patent Application Publication No. 2010-0322862. Protease cleavage sites can be predicted based on knowledge developed and known to those of skill in the art. For example, prediction of cleavage can be assessed at the website cbs.dtu.dk/services/ProP/. A useful class of peptides can consist of unblocked peptides and activatable peptides, which class excludes blocked peptides that are not activatable. The amino acids prior to the start of the CAR sequence can be chosen to form all or a portion of a protease recognition sequence.

Useful proteases include enzymes that cleave on the C terminal side of basic residues (the C terminal residue of CAR peptides is a basic residue) and enzymes that recognize sequence on the C terminal side of their cleavage site (thus allowing free choice of the C terminal sequence of the cleavage product). Examples of useful proteases include, for example, serine proteases (including, for example, plasmin and plasminogen activators), urokinase, proprotein convertases (see, for example, Duckert et al., Prediction of proprotein convertase cleavage sites Protein engineering Design and Selection 17(1):107-112 (2004)), furins, and carboxypeptidases, such as carboxypeptidase A (amino acids with aromatic or branched hydrocarbon side chains), glutamate-specific carboxypeptidase, proline-specific carboxypeptidase, and PSMA. Examples of enzymes that cleave on the C terminal side of basic residues include Arg-C protease (which cleaves on the C terminal side of arginine residues; Keil, Specificity of Proteolysis (Springer-Verlag, Berlin-Heidelberg-New York (1992)), clostripain (which cleaves on the C terminal side of arginine residues; Keil, 1992), enterokinase (which cleaves after the sequence -Asp-Asp-Asp-Asp-Lys-; SEQ ID NO:22), Factor Xa (which cleaves after the sequence -Gly-Arg-; Fujikawa et al., Activation of bovine factor X (Stuart factor): conversion of factor Xa alpha to factor Xa beta, Proc. Natl. Acad. Sci. 72: 3359-3363 (1975)), Lys-C (which cleaves on the C terminal side of lysine residues; Keil, 1992), thrombin (which cleaves on the C terminal side of arginine residues; Keil, 1992), trypsin (which cleaves on the C terminal side of arginine and lysine residues; Keil, 1992), serine proteases, proprotein convertases (such as PC1, PC2, PC3, PC4, PC5, PC6, PC7, PC8, furin, Pace, PACE4, Site 1 protease, SIP, SKI, NARC-1, PCSK1, PCSK2, PCSK3, PCSK4, PCSK5, PCSK6, PCSK7, PCSK8, and PCSK9), plasmin, and plasminogen activators. Examples of enzymes that recognize sequence on the C terminal side of their cleavage site include Asp-N endopeptidase (which cleaves on the N terminal side of aspartic acid; Keil, 1992) and carboxypeptidases such as carboxypeptidase A (which cleaves C-terminal residues except proline, lysine and arginine).

Examples of proteases are also described in Hook, Proteolytic and cellular mechanisms in prohormone and proprotein processing, RG Landes Company, Austin, Tex., USA (1998); Hooper et al., Biochem. J. 321: 265-279 (1997); Werb, Cell 91: 439-442 (1997); Wolfsberg et al., J. Cell Biol. 131: 275-278 (1995); Murakami and Etlinger, Biochem. Biophys. Res. Comm. 146: 1249-1259 (1987); Berg et al., Biochem. J. 307: 313-326 (1995); Smyth and Trapani, Immunology Today 16: 202-206 (1995); Talanian et al., J. Biol. Chem. 272: 9677-9682 (1997); and Thornberry et al., J. Biol. Chem. 272: 17907-17911 (1997).

As used herein, “activatable CendR element” refers to a CendR element having a molecule, moiety, nanoparticle, compound or other composition covalently coupled to the CendR element, such as to the terminal carboxyl group of the C-terminal element, where the molecule, moiety, nanoparticle, compound or other composition can block internalization and/or tissue penetration of the CendR composition, conjugate, molecule, protein, peptide, etc. and where the molecule, moiety, nanoparticle, compound or other composition can be removed (to expose the terminal carboxy group, for example). Activatable CendR elements are described in U.S. Patent Application Publication No. 2010-0322862, which is hereby incorporated by reference in its entirety and, in particular, for its description of CendR elements and activatable CendR elements.

Exopeptidases, such as carboxypeptidases, can be used to activate peptides. For example, carboxypeptidases are useful proteases for activating peptides. Carboxypeptidases remove the C-terminal amino acid from proteins and peptides. Carboxypeptidases can, within the limits of their substrate preferences, remove amino acids sequentially from a protein or peptide. Thus, for example, a carboxypeptidase could completely or nearly completely hydrolyze a protein of peptide. Because various carboxypeptidases have certain substrate preferences or limitations, and because carboxypeptidases generally only cleave peptide bonds, the presence of certain amino acids, modifications, and/or non-peptide bonds can control carboxypeptidase cleavage of a protein or peptide.

In the context of CAR peptides, the structure of and/or modifications to a protein, peptide or amino acid sequence comprising a CAR peptide can be chosen to result in cleavage by a carboxypeptidase ending at the C-terminal amino acid of the CAR peptide. This can be accomplished by, for example, using a bond between the C-terminal amino acid and the penultimate amino acid in the CAR peptide that can be protected from protease cleavage. For example, the bond can be a non-peptide bond or can include a modification, such as methylation. As another example, a D-amino acid can be used as the C-terminal amino acid, the penultimate amino acid, or both, in a CAR peptide. As another example, a D-amino acid can be used as the C-terminal amino acid in a CAR peptide. CAR peptides with limited use of D amino acids retain internalization and penetration activity. As another example, an amino acid that serves as a substrate for a carboxypeptidase can be located C-terminal to the C-terminal amino acid in the CAR peptide. For example, for a glutamate-specific carboxypeptidase such as PSMA, a glutamic acid amino acid can be placed adjacent to and C-terminal of the C-terminal amino acid in the CAR peptide and at the C-terminal end of the protein or peptide containing the CAR peptide. Other amino acid-specific (or preferring) carboxypeptidases can be used in similar ways. In these cases, the C-terminal amino acid in the CAR peptide should not be a substrate (or should be a disfavored substrate) for the carboxypeptidase.

Bonds and modifications to amino acids that can reduce or eliminate protease cleavage at a bond are known and can be used in the disclosed CAR peptides. For example, a variety of chemical modification techniques and moieties are described in, for example, U.S. Pat. Nos. 5,554,728, 6,869,932, 6,828,401, 6,673,580, 6,552,170, 6,420,339, U.S. Pat. Pub. 2006/0210526 and Intl. Pat. App. WO 2006/136586. Some examples of such modifications include peptide bond surrogates such as those described in Cudic and Stawikowski, Peptidomimetics: Fmoc Solid-Phase Pseudopeptide Synthesis, in Methods in Molecular Biology, vol. 294, 223-246 (2008), and chemical modifications, such as maleimide capping, polyethylene glycol (PEG) attachment, maleidification, acylation, alkylation, esterification, and amidification, to produce structural analogs of the peptide. These and other modifications are further described elsewhere herein.

The CAR peptide in a disclosed protein, peptide, amino acid sequence or CAR composition can be at any location in the protein, peptide, amino acid sequence or CAR composition. For example, the CAR peptide in a disclosed protein, peptide, amino acid sequence or CAR composition can be at a free C-terminal end or on the N-terminal side of the cleavage site in an activatable CAR peptide.

Also disclosed are methods of forming an activatable peptide, the method comprising causing a blocking group to be covalently coupled to a peptide, wherein a bond coupling the blocking group and the peptide is cleavable. Also disclosed are methods of forming an activatable peptide, the method comprising causing a blocking group to be covalently coupled to an amino acid sequence, wherein the amino acid sequence comprises the peptide, wherein a bond coupling the blocking group and the peptide is cleavable. Also disclosed are methods of forming an activatable peptide, the method comprising (a) selecting an amino acid sequence for internalization into a cell and/or penetration of tissue, wherein the amino acid sequence comprises a peptide, and (b) causing a blocking group to be covalently coupled to the peptide, wherein a bond coupling the blocking group and the peptide is cleavable. The blocking group covalently coupled to the peptide reduces or prevents internalization into a cell and/or penetration of tissue. The blocking group covalently coupled to the peptide can reduce or prevent internalization into a cell and/or penetration of tissue compared to the same peptide with no blocking group. For example, an amino acid sequence comprising CAR sequence-cleavage site-homing module can be made and then tested for activatability (via cleavage of the cleavage site, for example). For example, a pool of peptides having the amino acid sequence CARSKNK-XXXXXXXXXXXXXXXXX (SEQ ID NO:8) can be tested for homing and activatability. That is, such peptides can be identified by screens using libraries. The activatable peptide can comprise the selected amino acid sequence and the blocking group. The cell can be in a subject. The enzyme, cleaving agent, and/or cleaving conditions present in proximity to the cell of interest can be identified. The enzyme, cleaving agent, and/or cleaving conditions present in proximity to the cell of interest can be identified prior to forming the activatable peptide. The cleavable bond can be selected based on the enzyme that is present in proximity to the cell of interest. The cleavable bond can be selected based on the cleaving agent present at site where the peptide is delivered, homes, travels or accumulates, such as the cell of interest. The cleavable bond can be selected based on the cleaving conditions present at site where the peptide is delivered, homes, travels or accumulates, such as the cell of interest. The cleavable bond can be selected prior to forming the activatable peptide. The peptide can comprise a terminal carboxyl group, wherein the blocking group is coupled to the terminal carboxyl group.

“Internalization” refers to passage through a plasma membrane or other biological barrier. “Penetration” refers to passage into and through a cell, tissue, or other biological barrier. Penetration generally involves and includes internalization. The disclosed CAR peptides generally promote and allow both internalization (such as internalization into a cell) and penetration (such as tissue penetration). Reference to internalization or to penetration should be understood to refer to both internalization and penetration unless the context indicates otherwise (such as separate or distinct discussion and description of internalization into a cell and tissue penetration separately—the present paragraph is an example of such).

By “internalization into a cell” is meant that that CAR peptide is capable of penetrating the plasma membrane, thereby being internalized into the cell. This internalization can occur with, for example, 10, 20, 30, 40, 50, 60, 70, 80, 90, or 100% efficiency for a given CAR peptide and a given cell. tCAR peptides generally an promote, mediate, cause, enhance, etc. internalization; penetration; internalization into and/or through cells, tissue, or both; penetration into and/or through cells, tissue, or both; permeabilization of cells and/or tissues; or a combination. By “permeabilization” is meant promoting, mediating, causing, enhancing, etc. the ability and/or condition of cells and/or tissues to allow compositions, conjugates, molecules, etc. in proximity to the cells and/or tissues to enter and/or pass through the cells and/or tissues. Thus, the disclosed CAR proteins, peptides, conjugates, compositions, etc. can be said to permeabilize the cells and/or tissues. By “permeable” is meant the ability and/or condition of cells and/or tissues to allow compositions, conjugates, molecules, etc. in proximity to the cells and/or tissues to enter and/or pass through the cells and/or tissues.

As used herein, “tissue penetration” and “penetration of tissue” refer to passage into or through a tissue beyond or through the outer or a first layer of cells or through a tissue membrane. Such passage or penetration through tissue (which can also be referred to as extravasation and tissue penetration) can be a function of, for example, cell internalization and passage between cells in the tissue. Throughout this application, when the term “tissue penetration” is used, it is understood that such penetration can also extend to other barriers and suitable membranes found throughout the body, such as the blood brain barrier.

Cells that can internalize a CAR peptide can be identified by, for example, (a) exposing a cell to a CAR peptide; and (b) determining if the CAR peptide was internalized. The cell can be in an assay, for example. Any form or type of CAR peptide, CAR peptide, CAR protein, CAR conjugate, or CAR composition can be used in these methods.

A cell that can internalize a CAR peptide can be identified by, for example, (a) exposing a cell to a CAR peptide, and (b) determining if the CAR peptide was internalized. The cell can be in an assay. The CAR peptide can be coupled to a protein or peptide. The CAR peptide can be an activatable CAR peptide. The activatable CAR peptide can be activated before exposure to the cell. The activatable CAR peptide can be a protease-activatable CAR peptide. The protein or peptide can be circular. The protein or peptide can be linear. The CAR peptide can be at any position in the protein or peptide. The CAR peptide can be at other than an end of the protein or peptide. The CAR peptide can be at the C-terminal end of the protein or peptide. The CAR peptide can be at the N-terminal end of the protein or peptide. Any form or type of CAR peptide, CAR peptide, CAR protein, CAR conjugate, or CAR composition can be used in these methods.

A tissue that can be penetrated by a CAR peptide can be identified by, for example, (a) exposing a tissue to a CAR peptide, and (b) determining if the CAR peptide penetrated the tissue. The CAR peptide can be coupled to a protein or peptide. The CAR peptide can be an activatable CAR peptide. The activatable CAR peptide can be activated before exposure to the tissue. The activatable CAR peptide can be a protease-activatable CAR peptide. The protein or peptide can be circular. The protein or peptide can be linear. The CAR peptide can be at any position in the protein or peptide. The CAR peptide can be at other than an end of the protein or peptide. The CAR peptide can be at the C-terminal end of the protein or peptide. The CAR peptide can be at the N-terminal end of the protein or peptide. Any form or type of CAR peptide, CAR peptide, CAR protein, CAR conjugate, or CAR composition can be used in these methods.

The CAR peptide can have a length of up to 10, 20, 30, 40, 50, 100, 150, 200, 250, 300, 400, 500, 1000 or 2000 residues. In particular embodiments, a CAR peptide can have a length of at least 7, 8, 9, 10, 15, 20, 25, 30, 40, 50, 60, 70, 80, 90, 100 or 200 residues. In further embodiments, a CAR peptide can have a length of 7 to 200 residues, 7 to 100 residues, 7 to 90 residues, 7 to 80 residues, 7 to 70 residues, 7 to 60 residues, 7 to 50 residues, 7 to 40 residues, 7 to 30 residues, 7 to 20 residues, 7 to 15 residues, 7 to 10 residues, 8 to 200 residues, 8 to 100 residues, 8 to 90 residues, 8 to 80 residues, 8 to 70 residues, 8 to 60 residues, 8 to 50 residues, 8 to 40 residues, 8 to 30 residues, 8 to 20 residues, 8 to 15 residues, 8 to 10 residues, 9 to 200 residues, 9 to 100 residues, 9 to 90 residues, 9 to 80 residues, 9 to 70 residues, 9 to 60 residues, 9 to 50 residues, 9 to 40 residues, 9 to 30 residues, 9 to 20 residues, 9 to 15 residues, 9 to 10 residues, 10 to 200 residues, 10 to 100 residues, 10 to 90 residues, 10 to 80 residues, 10 to 70 residues, 10 to 60 residues, 10 to 50 residues, 10 to 40 residues, 10 to 30 residues, 10 to 20 residues, 10 to 15 residues, 15 to 200 residues, 15 to 100 residues, 15 to 90 residues, 15 to 80 residues, 15 to 70 residues, 15 to 60 residues, 15 to 50 residues, 15 to 40 residues, 15 to 30 residues, 15 to 20 residues, 20 to 200 residues, 20 to 100 residues, 20 to 90 residues, 20 to 80 residues, 20 to 70 residues, 20 to 60 residues, 20 to 50 residues, 20 to 40 residues or 20 to 30 residues. As used herein, the term “residue” refers to an amino acid or amino acid analog.

A protein or peptide containing a CAR peptide can have a length of up to 50, 100, 150, 200, 250, 300, 400, 500, 1000 or 2000 residues. In particular embodiments, the protein or peptide portion of a CAR composition can have a length of at least 10, 20, 30, 40, 50, 60, 70, 80, 90, 100 or 200 residues. In further embodiments, the protein or peptide containing a CAR peptide can have a length of 7 to 200 residues, 7 to 100 residues, 7 to 90 residues, 7 to 80 residues, 7 to 70 residues, 7 to 60 residues, 7 to 50 residues, 7 to 40 residues, 7 to 30 residues, 7 to 20 residues, 7 to 15 residues, 7 to 10 residues, 8 to 200 residues, 8 to 100 residues, 8 to 90 residues, 8 to 80 residues, 8 to 70 residues, 8 to 60 residues, 8 to 50 residues, 8 to 40 residues, 8 to 30 residues, 8 to 20 residues, 8 to 15 residues, 8 to 10 residues, 9 to 200 residues, 9 to 100 residues, 9 to 90 residues, 9 to 80 residues, 9 to 70 residues, 9 to 60 residues, 9 to 50 residues, 9 to 40 residues, 9 to 30 residues, 9 to 20 residues, 9 to 15 residues, 9 to 10 residues, 10 to 200 residues, 10 to 100 residues, 10 to 90 residues, 10 to 80 residues, 10 to 70 residues, 10 to 60 residues, 10 to 50 residues, 10 to 40 residues, 10 to 30 residues, 10 to 20 residues, 10 to 15 residues, 15 to 200 residues, 15 to 100 residues, 15 to 90 residues, 15 to 80 residues, 15 to 70 residues, 15 to 60 residues, 15 to 50 residues, 15 to 40 residues, 15 to 30 residues, 15 to 20 residues, 20 to 200 residues, 20 to 100 residues, 20 to 90 residues, 20 to 80 residues, 20 to 70 residues, 20 to 60 residues, 20 to 50 residues, 20 to 40 residues or 20 to 30 residues.

The CAR conjugate can have a length of up to 50, 100, 150, 200, 250, 300, 400, 500, 1000 or 2000 residues. In particular embodiments, a CAR conjugate can have a length of at least 10, 20, 30, 40, 50, 60, 70, 80, 90, 100 or 200 residues. In further embodiments, a CAR conjugate can have a length of 10 to 200 residues, 10 to 100 residues, 10 to 90 residues, 10 to 80 residues, 10 to 70 residues, 10 to 60 residues, 10 to 50 residues, 10 to 40 residues, 10 to 30 residues, 10 to 20 residues, 10 to 15 residues, 15 to 200 residues, 15 to 100 residues, 15 to 90 residues, 15 to 80 residues, 15 to 70 residues, 15 to 60 residues, 15 to 50 residues, 15 to 40 residues, 15 to 30 residues, 15 to 20 residues, 20 to 200 residues, 20 to 100 residues, 20 to 90 residues, 20 to 80 residues, 20 to 70 residues, 20 to 60 residues, 20 to 50 residues, 20 to 40 residues or 20 to 30 residues.

The protein or peptide portion of a CAR composition can have a length of up to 50, 100, 150, 200, 250, 300, 400, 500, 1000 or 2000 residues. In particular embodiments, the protein or peptide portion of a CAR composition can have a length of at least 10, 20, 30, 40, 50, 60, 70, 80, 90, 100 or 200 residues. In further embodiments, the protein or peptide portion of a CAR composition can have a length of 7 to 200 residues, 7 to 100 residues, 7 to 90 residues, 7 to 80 residues, 7 to 70 residues, 7 to 60 residues, 7 to 50 residues, 7 to 40 residues, 7 to 30 residues, 7 to 20 residues, 7 to 15 residues, 7 to 10 residues, 8 to 200 residues, 8 to 100 residues, 8 to 90 residues, 8 to 80 residues, 8 to 70 residues, 8 to 60 residues, 8 to 50 residues, 8 to 40 residues, 8 to 30 residues, 8 to 20 residues, 8 to 15 residues, 8 to 10 residues, 9 to 200 residues, 9 to 100 residues, 9 to 90 residues, 9 to 80 residues, 9 to 70 residues, 9 to 60 residues, 9 to 50 residues, 9 to 40 residues, 9 to 30 residues, 9 to 20 residues, 9 to 15 residues, 9 to 10 residues, 10 to 200 residues, 10 to 100 residues, 10 to 90 residues, 10 to 80 residues, 10 to 70 residues, 10 to 60 residues, 10 to 50 residues, 10 to 40 residues, 10 to 30 residues, 10 to 20 residues, 10 to 15 residues, 15 to 200 residues, 15 to 100 residues, 15 to 90 residues, 15 to 80 residues, 15 to 70 residues, 15 to 60 residues, 15 to 50 residues, 15 to 40 residues, 15 to 30 residues, 15 to 20 residues, 20 to 200 residues, 20 to 100 residues, 20 to 90 residues, 20 to 80 residues, 20 to 70 residues, 20 to 60 residues, 20 to 50 residues, 20 to 40 residues or 20 to 30 residues.

The CAR composition can have a length of up to 50, 100, 150, 200, 250, 300, 400, 500, 1000 or 2000 residues. In particular embodiments, a CAR composition can have a length of at least 10, 20, 30, 40, 50, 60, 70, 80, 90, 100 or 200 residues. In further embodiments, a CAR composition can have a length of 10 to 200 residues, 10 to 100 residues, 10 to 90 residues, 10 to 80 residues, 10 to 70 residues, 10 to 60 residues, 10 to 50 residues, 10 to 40 residues, 10 to 30 residues, 10 to 20 residues, 10 to 15 residues, 15 to 200 residues, 15 to 100 residues, 15 to 90 residues, 15 to 80 residues, 15 to 70 residues, 15 to 60 residues, 15 to 50 residues, 15 to 40 residues, 15 to 30 residues, 15 to 20 residues, 20 to 200 residues, 20 to 100 residues, 20 to 90 residues, 20 to 80 residues, 20 to 70 residues, 20 to 60 residues, 20 to 50 residues, 20 to 40 residues or 20 to 30 residues.

CAR (and other) peptides can be stabilized against proteolysis. For example, the stability and activity of peptides, such as tumor-homing peptides CREKA (Simberg et al., 2007), by protecting some of the peptide bonds with N-methylation or C-methylation. The most important bond to protect in order to enhance activity is the R-G bond because it would prevent a cleavage that would inactivate both the integrin-binding and CAR activities. Accessory peptides and homing peptides can also or similarly be stabilized against proteolysis.

CAR peptides can be made in the form of stabilized peptides and/or formulated as long-circulating forms. For example, a polyethylene glycol conjugate can be used. CAR peptides can also be administered over a period of time. For example, CAR peptides can be delivered with an osmotic pump. This can extend the permeability of the target cells and tissues. Modified forms of CAR peptides can be used. For example, CAR peptides can be methylated (which can stabilize the peptides against proteolysis). Stability against cleavage is desirable, except for bonds to be cleaved to activate CAR peptides. Modifications to CAR peptides generally should leave them functional or capable of function after activation.

It is understood that there are numerous amino acid and peptide analogs which can be incorporated into the disclosed CAR compositions, conjugates, molecules, proteins, peptides, and elements. For example, there are numerous D amino acids or other non-natural amino acids which can be used. The opposite stereoisomers of naturally occurring peptides are disclosed, as well as the stereo isomers of peptide analogs. These amino acids can readily be incorporated into polypeptide chains by chemical synthesis or by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO—(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appin, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides as long as activity is preserved. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

Disclosed are polyfunctional CAR compositions which, in addition to the CAR peptide, contain, for example, an accessory peptide, an accessory peptide fused to the CAR peptide, an accessory molecule covalently coupled to or non-covalently associated with the CAR peptide, a homing peptide fused to the CAR peptide, and/or a homing molecule covalently coupled to or non-covalently associated with the CAR peptide. Additional compounds having separate functions can be added to the composition. Such polyfunctional conjugates have at least two functions conferred by different portions of the composition and can, for example, display anti-angiogenic activity or pro-apoptotic activity in addition to selective homing activity.

As used herein, the term “peptide” is used broadly to mean peptides, proteins, fragments of proteins and the like. The term “peptidomimetic,” as used herein, means a peptide-like molecule that has the activity of the peptide upon which it is structurally based. Such peptidomimetics include chemically modified peptides, peptide-like molecules containing non-naturally occurring amino acids, and peptoids and have an activity such as that from which the peptidomimetic is derived (see, for example, Goodman and Ro, Peptidomimetics for Drug Design, in “Burger's Medicinal Chemistry and Drug Discovery” Vol. 1 (ed. M. E. Wolff; John Wiley & Sons 1995), pages 803-861).

Protein variants and derivatives are well understood by those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 5 and 6 and are referred to as conservative substitutions.

TABLE 5
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 6
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 6, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NOs:10 and 4 set forth particular sequences of CAR. Specifically disclosed are variants of these and other peptides herein disclosed which have at least, 70% or 75% or 80% or 85% or 90% or 95% homology to the stated sequence. Wherein a sequence is said to have at least about 70% sequence identity, it is understood to also have at least about 75%, 80%, 85%, 90%, 92%, 92%, 93%, 94%, 95%, 96%, 97%, 98%, 99%, or 100% sequence identity.

A design principle for homing peptides has been developed that combines three functions: tissue-specific homing, spreading within the target tissue, and internalization into cells in that tissue. These peptides contain both a tissue-specific homing sequence and a tissue-penetrating and internalizing motif embodied in a CAR peptide.

The disclosed compounds are useful tools for introducing materials into the target tissues. They can allow disease-specific or cell type and tissue-specific targeting of diagnostic and therapeutic compounds to increase efficacy and decrease side effects. The principles disclosed herein are applicable to any cells or tissues for which specific homing peptides can be obtained.

Studies have revealed extensive molecular heterogeneity in the vasculature of different normal tissues. In addition, pathological lesions, such as tumors, impose their own changes on the vasculature. This system of molecular markers can be referred to as ‘vascular zip codes’ (Ruoslahti, 2004). The zip codes enable docking-based (synaphic) targeting to selectively deliver diagnostics and therapeutics into a specific tissue. This approach can produce greater efficacy and diminished side effects. The targeted delivery principle has been established, particularly in cancer: targeting of radioisotopes to leukemic cells with antibodies is an established therapy, and several products aimed at diagnosis and treatment of solid tumors are in clinical trials; many of them use early generation tumor-homing peptides or their derivatives. However, one issue in making the synaphic delivery more generally useful is that efficacy has tended to be low. It has been realized that it may be more effective to target the delivery to blood vessels because their inner endothelial lining is readily available to circulating probes, whereas penetration into tumor parenchyma has been a problem in the past (Jain, 1990). Thus, while it has been easy to demonstrate binding of the targeted material to the target vessels, a substantially higher concentration of the material in the target tissue has not necessarily been achieved (e.g. Liu et al., 2007).

The disclosed peptides can be validated by, for example, testing in vitro cell binding and internalization, and in vivo homing. Synthetic peptides can be used to show that the activities associated with the selected phage are reproduced by the peptide the phage displays. Techniques for this are well known (e.g. Zhang et al., 2005; Simberg et al., 2007; Karmali et al., 2008). The peptides generally can be labeled with a fluorophore to allow detection in tissues, and both the free peptide and a multimeric conjugate on nanoparticles (which more closely resembles the multivalent presentation on phage) can be tested.

A variety of homing molecules can be used in the disclosed compositions, conjugates and methods. Such homing molecules include, without limitation, peptides as disclosed herein. The disclosed compounds, compositions, conjugates and methods can include or use the disclosed homing molecules in various forms, including peptides and peptidomimetics as disclosed. For convenience of expression, in many places herein the use or inclusion of peptides will be recited. It is understood that, in such cases, it is considered that homing molecules in various forms can also be used or included in the same or similar ways as is described in terms of peptides, and such use and inclusion is specifically contemplated and disclosed thereby.

The term “homing molecule” as used herein, means any molecule that selectively homes in vivo to specific cells or specific tissue in preference to normal tissue. Similarly, the term “homing peptide” or “homing peptidomimetic” means a peptide that selectively homes in vivo to specific cells or specific tissue in preference to normal tissue. It is understood that a homing molecule that selectively homes in vivo to specific cells or specific tissue or can exhibit preferential homing to r specific cells or specific tissue.

By “selectively homes” is meant that in vivo, the homing molecule binds preferentially to the target as compared to non-target. For example, the homing molecule can bind preferentially to wounded tissue, as compared to non-wounded tissue. Selective homing to, for example, wounded tissue generally is characterized by at least a two-fold greater localization within wounded tissue, as compared to several tissue types of non-wounded tissue. A homing molecule can be characterized by, for example, 5-fold, 10-fold, 20-fold or more preferential localization to the target as compared to one or more non-targets. For example, a homing molecule can be characterized by, for example, 5-fold, 10-fold, 20-fold or more preferential localization to wound vasculature as compared to vasculature of several or many tissue types of non-wounded tissue, or as compared to vasculature of most or all non-wounded tissue. As another example, a homing molecule can be characterized by, for example, 5-fold, 10-fold, 20-fold or more preferential localization to wounded tissue as compared to several or many tissue types of non-wounded tissue, or as compared to-most or all non-wounded tissue. Thus, it is understood that, in some cases, a homing molecule homes, in part, to one or more normal organs in addition to homing to the target tissue. Selective homing can also be referred to as targeting. The molecules, proteins, cells, tissues, etc. that are targeted by homing molecules can be referred to as targeted molecules, proteins, cells, tissues, etc.

Binding in the context of a homing molecule recognizing and/or binding to its target can refer to both covalent and non-covalent binding, for example where a homing molecule can bind, attach or otherwise couple to its target by covalent and/or non-covalent binding. Binding can be either high affinity or low affinity, preferably high affinity. Examples of binding forces that can be useful include, but are not limited to, covalent bonds, dipole interactions, electrostatic forces, hydrogen bonds, hydrophobic interactions, ionic bonds, and/or van der Waals forces. This binding can occur in addition to that binding which occurs with the CAR peptide.

Surface molecules can be associated with and arranged in the compositions in a variety of configurations. In some forms, surface molecules can be associated with, conjugated to, and/or covalently coupled to a plurality of homing molecules, a plurality of cargo molecules, or both. In some forms, surface molecules can be associated with, conjugated to, and/or covalently coupled to a plurality of homing molecules, wherein the homing molecules can be associated with, conjugated to, and/or covalently coupled to a plurality of cargo molecules. In some forms, surface molecules can be associated with, conjugated to, and/or covalently coupled to a plurality of cargo molecules, wherein the cargo molecules can be associated with, conjugated to, and/or covalently coupled to a plurality of homing molecules. Combinations of these combinations can also be used.

The surface molecules, alternatively referred to as a surface particles, disclosed herein can be conjugated with homing molecules and cargo molecules in such a way that the composition is delivered to a target. The surface molecule can be any substance that can be used with the homing molecules and cargo molecules, and is not restricted by size or substance. Examples include, but are not limited to, nanoparticles (such as iron oxide nanoparticles or albumin nanoparticles), liposomes, small organic molecules, microparticles, or microbubbles, such as fluorocarbon microbubbles. The term surface molecule is used to identify a component of the disclosed composition but is not intended to be limiting. In particular, the disclosed surface molecules are not limited to substances, compounds, compositions, particles or other materials composed of a single molecule. Rather, the disclosed surface molecules are any substance(s), compound(s), composition(s), particle(s) and/or other material(s) that can be conjugated with a plurality of homing molecules and cargo molecules such that at least some of the homing molecules and/or cargo molecules are presented and/or accessible on the surface of the surface molecule. A variety of examples of suitable surface molecules are described and disclosed herein.

The surface molecule can be detectable, or can be a therapeutic agent such as iRGD, RGD, or Abraxane™. The section herein which discusses cargo molecules and moieties that can be detectable or therapeutic also applies to the surface molecule.

The term “nanoparticle” refers to a nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, nanoworms, fullerene-like materials, inorganic nanotubes, dendrimers (such as with covalently attached metal chelates), nanofibers, nanohoms, nano-onions, nanorods, nanoropes and quantum dots. A nanoparticle can produce a detectable signal, for example, through absorption and/or emission of photons (including radio frequency and visible photons) and plasmon resonance.

Microspheres (or microbubbles) can also be used with the methods disclosed herein. Microspheres containing chromophores have been utilized in an extensive variety of applications, including photonic crystals, biological labeling, and flow visualization in microfluidic channels. See, for example, Y. Lin, et al., Appl. Phys Lett. 2002, 81, 3134; D. Wang, et al., Chem. Mater. 2003, 15, 2724; X. Gao, et al., J. Biomed. Opt. 2002, 7, 532; M. Han, et al., Nature Biotechnology. 2001, 19, 631; V. M. Pai, et al., Mag. & Magnetic Mater. 1999, 194, 262, each of which is incorporated by reference in its entirety. Both the photostability of the chromophores and the monodispersity of the microspheres can be important.

Nanoparticles, such as, for example, metal nanoparticles, metal oxide nanoparticles, or semiconductor nanocrystals can be incorporated into microspheres. The optical, magnetic, and electronic properties of the nanoparticles can allow them to be observed while associated with the microspheres and can allow the microspheres to be identified and spatially monitored. For example, the high photostability, good fluorescence efficiency and wide emission tunability of colloidally synthesized semiconductor nanocrystals can make them an excellent choice of chromophore. Unlike organic dyes, nanocrystals that emit different colors (i.e. different wavelengths) can be excited simultaneously with a single light source. Colloidally synthesized semiconductor nanocrystals (such as, for example, core-shell CdSe/ZnS and CdS/ZnS nanocrystals) can be incorporated into microspheres. The microspheres can be monodisperse silica microspheres.

The nanoparticle can be a metal nanoparticle, a metal oxide nanoparticle, or a semiconductor nanocrystal. The metal of the metal nanoparticle or the metal oxide nanoparticle can include titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, scandium, yttrium, lanthanum, a lanthanide series or actinide series element (e.g., cerium, praseodymium, neodymium, promethium, samarium, europium, gadolinium, terbium, dysprosium, holmium, erbium, thulium, ytterbium, lutetium, thorium, protactinium, and uranium), boron, aluminum, gallium, indium, thallium, silicon, germanium, tin, lead, antimony, bismuth, polonium, magnesium, calcium, strontium, and barium. In certain embodiments, the metal can be iron, ruthenium, cobalt, rhodium, nickel, palladium, platinum, silver, gold, cerium or samarium. The metal oxide can be an oxide of any of these materials or combination of materials. For example, the metal can be gold, or the metal oxide can be an iron oxide, a cobalt oxide, a zinc oxide, a cerium oxide, or a titanium oxide. Preparation of metal and metal oxide nanoparticles is described, for example, in U.S. Pat. Nos. 5,897,945 and 6,759,199, each of which is incorporated by reference in its entirety.

The nanoparticles can be comprised of cargo molecules and a carrier protein (such as albumin). Such nanoparticles are useful, for example, to deliver hydrophobic or poorly soluble compounds. Nanoparticles of poorly water soluble drugs (such as taxane) have been disclosed in, for example, U.S. Pat. Nos. 5,916,596; 6,506,405; and 6,537,579 and also in U.S. Pat. Pub. No. 2005/0004002A1.

In forms, the nanoparticles can have an average or mean diameter of no greater than about 1000 nanometers (nm), such as no greater than about any of 900, 800, 700, 600, 500, 400, 300, 200, and 100 nm. In some forms, the average or mean diameters of the nanoparticles can be no greater than about 200 nm. In some forms, the average or mean diameters of the nanoparticles can be no greater than about 150 nm. In some forms, the average or mean diameters of the nanoparticles can be no greater than about 100 nm. In some forms, the average or mean diameter of the nanoparticles can be about 20 to about 400 nm. In some forms, the average or mean diameter of the nanoparticles can be about 40 to about 200 nm. In some embodiments, the nanoparticles are sterile-filterable.

The nanoparticles can be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

Examples of suitable carrier proteins include proteins normally found in blood or plasma, which include, but are not limited to, albumin, immunoglobulin including IgA, lipoproteins, apolipoprotein B, alpha-acid glycoprotein, beta-2-macroglobulin, thyroglobulin, transferin, fibronectin, factor VII, factor VIII, factor IX, factor X, and the like. In some embodiments, the carrier protein is non-blood protein, such as casein, alpha-lactalbumin, and beta-lactoglobulin. The carrier proteins may either be natural in origin or synthetically prepared. In some embodiments, the pharmaceutically acceptable carrier comprises albumin, such as human serum albumin. Human serum albumin (HSA) is a highly soluble globular protein of Mᵣ 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see, e.g., Tullis, JAMA 237:355-360, 460-463 (1977)) and Houser et al., Surgery, Gynecology and Obstetrics, 150:811-816 (1980)) and in conjunction with exchange transfusion in the treatment of neonatal hyperbilirubinemia (see, e.g., Finlayson, Seminars in Thrombosis and Hemostasis, 6:85-120 (1980)). Other albumins are contemplated, such as bovine serum albumin. Use of such non-human albumins could be appropriate, for example, in the context of use of these compositions in non-human mammals, such as the veterinary (including domestic pets and agricultural context).

Carrier proteins (such as albumin) in the composition generally serve as a carrier for the hydrophobic cargo molecules, i.e., the carrier protein in the composition makes the cargo molecules more readily suspendable in an aqueous medium or helps maintain the suspension as compared to compositions not comprising a carrier protein. This can avoid the use of toxic solvents (or surfactants) for solubilizing the cargo molecules, and thereby can reduce one or more side effects of administration of the cargo molecules into an individual (such as a human). Thus, in some embodiments, the composition described herein can be substantially free (such as free) of surfactants, such as Cremophor (including Cremophor EL® (BASF)). In some embodiments, the composition can be substantially free (such as free) of surfactants. A composition is “substantially free of Cremophor” or “substantially free of surfactant” if the amount of Cremophor or surfactant in the composition is not sufficient to cause one or more side effect(s) in an individual when the composition is administered to the individual.

The amount of carrier protein in the composition described herein will vary depending on other components in the composition. In some embodiments, the composition, and/or CAR composition can comprise a carrier protein in an amount that is sufficient to stabilize the cargo molecules in an aqueous suspension, for example, in the form of a stable colloidal suspension (such as a stable suspension of nanoparticles). In some embodiments, the carrier protein is in an amount that reduces the sedimentation rate of the cargo molecules in an aqueous medium. For particle-containing compositions, the amount of the carrier protein also depends on the size and density of nanoparticles of the cargo molecules.

Methods of making nanoparticle compositions are known in the art. For example, nanoparticles containing cargo molecules and carrier protein (such as albumin) can be prepared under conditions of high shear forces (e.g., sonication, high pressure homogenization, or the like). These methods are disclosed in, for example, U.S. Pat. Nos. 5,916,596; 6,506,405; and 6,537,579 and also in U.S. Pat. Pub. No. 2005/0004002A1.

Briefly, the hydrophobic carrier molecules can be dissolved in an organic solvent, and the solution can be added to a human serum albumin solution. The mixture is subjected to high pressure homogenization. The organic solvent can then be removed by evaporation. The dispersion obtained can be further lyophilized. Suitable organic solvent include, for example, ketones, esters, ethers, chlorinated solvents, and other solvents known in the art. For example, the organic solvent can be methylene chloride and chloroform/ethanol (for example with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1).

The nanoparticle can also be, for example, a heat generating nanoshell. As used herein, “nanoshell” is a nanoparticle having a discrete dielectric or semi-conducting core section surrounded by one or more conducting shell layers. U.S. Pat. No. 6,530,944 is hereby incorporated by reference herein in its entirety for its teaching of the methods of making and using metal nanoshells. Targeting molecules can be attached to the disclosed compositions and/or carriers. For example, the targeting molecules can be antibodies or fragments thereof, ligands for specific receptors, or other proteins specifically binding to the surface of the cells to be targeted.

As used herein, the term “dendrimer” refers to repeatedly branched and roughly spherical molecules. A dendrimer is typically symmetric around a core and usually adopts a spherical three-dimensional morphology. Dendrimers generally contain three major portions: a core, an inner shell and an outer shell. Dendrimers can be synthesized to have different and varying functionality in each of the major portions in order to control such variables as solubility, thermal stability and attachment of compounds suitable for particular applications.

Dendrimers can be macromolecules having well-defined hyperbranched structures. Peptide dendrimers are radially branched macromolecules that contain a peptidyl branching core and/or peripheral peptide chains, and they can be divided into three categories. One category consists of “grafted” peptide dendrimers, having peptides only as surface functionalities. The second category is peptide dendrimers that composed entirely of amino acids. The third are dendrimers utilizing amino acids in the branching core and surface functional groups, but having non-peptide branching units. Peptide dendrimers can be synthesized using either divergent or convergent approach, and the availability of solid-phase combinatorial methods enables large libraries of peptide dendrimers to be produced and screened for desired properties.

Dendrimer variants of SEQ ID NO:10 or SEQ ID NO:4 can be synthesized. For example, a CAR dendrimer containing 8 CAR residues on a polyamidoamine (PAMAM) core can be constructed. Other cores such as Poly (ethylene glycol) can also be used to create dendrimer variants. These dendrimer variants can have dozens, hundreds, or even thousands of CAR peptide residues on the surface of the dendrimer to provide enhanced functional characteristics. These dendrimers can contain CAR alone for disease selective homing, cell penetration and delivery of co-administered bioactive agents or contain a bioactive agent within the dendrimer for targeted delivery.

“Liposome” as the term is used herein refers to a structure comprising an outer lipid bi- or multi-layer membrane surrounding an internal aqueous space. Liposomes can be used to package any biologically active agent for delivery to cells.

Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 μm. These MLVs were first described by Bangham, et al., J Mol. Biol. 13:238-252 (1965). In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other forms of mechanical shearing. There are also techniques by which MLVs can be reduced both in size and in number of lamellae, for example, by pressurized extrusion (Barenholz, et al., FEBS Lett. 99:210-214 (1979)).

Liposomes can also take the form of unilamnellar vesicles, which are prepared by more extensive sonication of MLVs, and consist of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 μm. There are several well-known techniques for making unilamellar vesicles. In Papahadjopoulos, et al., Biochim et Biophys Acta 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973) and the ether injection technique of Deamer, et al., Biochim et Biophys Acta 443:629-634 (1976). These methods involve the rapid injection of an organic solution of lipids into a buffer solution, which results in the rapid formation of unilamellar liposomes. Another technique for making ULVs is taught by Weder, et al. in “Liposome Technology”, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos, et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septum. Still yet another type of liposomes are oligolamellar vesicles (“OLVs”), which have a large center compartment surrounded by several peripheral lipid layers. These vesicles, having a diameter of 2-15 μm, are described in Callo, et al., Cryobiology 22(3):251-267 (1985).

Mezei, et al., U.S. Pat. Nos. 4,485,054 and 4,761,288 also describe methods of preparing lipid vesicles. More recently, Hsu, U.S. Pat. No. 5,653,996 describes a method of preparing liposomes utilizing aerosolization and Yiournas, et al., U.S. Pat. No. 5,013,497 describes a method for preparing liposomes utilizing a high velocity-shear mixing chamber. Methods are also described that use specific starting materials to produce ULVs (Wallach, et al., U.S. Pat. No. 4,853,228) or OLVs (Wallach, U.S. Pat. Nos. 5,474,848 and 5,628,936).

A comprehensive review of all the aforementioned lipid vesicles and methods for their preparation are described in “Liposome Technology”, ed. G. Gregoriadis, CRC Press Inc., Boca Raton, Fla., Vol. I, II & III (1984). This and the aforementioned references describing various lipid vesicles suitable for use in the disclosed compositions are incorporated herein by reference.

“Micelle” as used herein refers to a structure comprising an outer lipid monolayer. Micelles can be formed in an aqueous medium when the Critical Micelle Concentration (CMC) is exceeded. Small micelles in dilute solution at approximately the critical micelle concentration (CMC) are generally believed to be spherical. However, under other conditions, they may be in the shape of distorted spheres, disks, rods, lamellae, and the like. Micelles formed from relatively low molecular weight amphiphile molecules can have a high CMC so that the formed micelles dissociate rather rapidly upon dilution. If this is undesired, amphiphile molecules with large hydrophobic regions can be used. For example, lipids with a long fatty acid chain or two fatty acid chains, such as phospholipids and sphingolipids, or polymers, specifically block copolymers, can be used.

Polymeric micelles have been prepared that exhibit CMCs as low as 10⁻⁶ M (molar). Thus, they tend to be very stable while at the same time showing the same beneficial characteristics as amphiphile micelles. Any micelle-forming polymer presently known in the art or as such may become known in the future may be used in the disclosed compositions and methods. Examples of micelle-forming polymers include, without limitation, methoxy poly(ethylene glycol)-b-poly(∈-caprolactone), conjugates of poly(ethylene glycol) with phosphatidyl-ethanolamine, poly(ethylene glycol)-b-polyesters, poly(ethylene glycol)-b-poly(L-aminoacids), poly(N-vinylpyrrolidone)-bl-poly(orthoesters), poly(N-vinylpyrrolidone)-b-polyanhydrides and poly(N-vinylpyrrolidone)-b-poly(alkyl acrylates).

Micelles can be produced by processes conventional in the art. Examples of such are described in, for example, Liggins (Liggins, R. T. and Burt, H. M., “Polyether-polyester diblock copolymers for the preparation of paclitaxel loaded polymeric micelle formulations.” Adv. Drug Del. Rev. 54: 191-202, (2002)); Zhang, et al. (Zhang, X. et al., “Development of amphiphilic dibiock copolymers as micellar carriers of taxol.” Int. J. Pharm. 132: 195-206, (1996)); and Churchill (Churchill, J. R., and Hutchinson, F. G., “Biodegradable amphipathic copolymers.” U.S. Pat. No. 4,745,160, (1988)). In one such method, polyether-polyester block copolymers, which are amphipathic polymers having hydrophilic (polyether) and hydrophobic (polyester) segments, are used as micelle forming carriers.

Another type of micelle can be formed using, for example, AB-type block copolymers having both hydrophilic and hydrophobic segments, as described in, for example, Tuzar (Tuzar, Z. and Kratochvil, P., “Block and graft copolymer micelles in solution.”, Adv. Colloid Interface Sci. 6:201-232, (1976)); and Wilhelm, et al. (Wilhelm, M. et al., “Poly(styrene-ethylene oxide) block copolymer micelle formation in water: a fluorescence probe study.”, Macromolecules 24:1033-1040 (1991)). These polymeric micelles are able to maintain satisfactory aqueous stability. These micelles, in the range of approximately <200 nm in size, are effective in reducing non-selective RES scavenging and show enhanced permeability and retention.

Further, U.S. Pat. No. 5,929,177 to Kataoka, et al. describes a polymeric molecule which is usable as, inter alia, a drug delivery carrier. The micelle is formed from a block copolymer having functional groups on both of its ends and which comprises hydrophilic/hydrophobic segments. The polymer functional groups on the ends of the block copolymer include amino, carboxyl and mercapto groups on the .alpha.-terminal and hydroxyl, carboxyl group, aldehyde group and vinyl group on the .omega.-terminal. The hydrophilic segment comprises polyethylene oxide, while the hydrophobic segment is derived from lactide, lactone or (meth)acrylic acid ester.

Further, for example, poly(D,L-lactide)-b-methoxypolyethylene glycol (MePEG:PDLLA) diblock copolymers can be made using MePEG 1900 and 5000. The reaction can be allowed to proceed for 3 hr at 160° C., using stannous octoate (0.25%) as a catalyst. However, a temperature as low as 130° C. can be used if the reaction is allowed to proceed for about 6 hr, or a temperature as high as 190° C. can be used if the reaction is carried out for only about 2 hr.

As another example, N-isopropylacrylamide (“IPAAm”) (Kohjin, Tokyo, Japan) and dimethylacrylamide (“DMAAm”) (Wako Pure Chemicals, Tokyo, Japan) can be used to make hydroxyl-terminated poly(IPAAm-co-DMAAm) in a radical polymerization process, using the method of Kohori, F. et al. (1998). (Kohori, F. et al., “Preparation and characterization of thermally Responsive block copolymer micelles comprising poly(N-isopropylacrylamide-b-D,L-lactide).” J. Control. Rel. 55: 87-98, (1998)). The obtained copolymer can be dissolved in cold water and filtered through two ultrafiltration membranes with a 10,000 and 20,000 molecular weight cut-off. The polymer solution is first filtered through a 20,000 molecular weight cut-off membrane. Then the filtrate was filtered again through a 10,000 molecular weight cut-off membrane. Three molecular weight fractions can be obtained as a result, a low molecular weight, a middle molecular weight, and a high molecular weight fraction. A block copolymer can then be synthesized by a ring opening polymerization of D,L-lactide from the terminal hydroxyl group of the poly(IPAAm-co-DMAAm) of the middle molecular weight fraction. The resulting poly(IPAAm-co-DMAAm)-b-poly(D,L-lactide) copolymer can be purified as described in Kohori, F. et al. (1999). (Kohori, F. et al., “Control of adriamycin cytotoxic activity using thermally responsive polymeric micelles composed of poly(N-isopropylacrylamide-co-N,N-dimethylacrylamide)-b-poly(D,L-lacide).-”, Colloids Surfaces B: Biointerfaces 16: 195-205, (1999)).

Examples of block copolymers from which micelles can be prepared which can be used to coat a support surface are found in U.S. Pat. No. 5,925,720, to Kataoka, et al., U.S. Pat. No. 5,412,072 to Sakarai, et al., U.S. Pat. No. 5,410,016 to Kataoka, et al., U.S. Pat. No. 5,929,177 to Kataoka, et al., U.S. Pat. No. 5,693,751 to Sakurai, et al., U.S. Pat. No. 5,449,513 to Yokoyama, et al., WO 96/32434, WO 96/33233 and WO 97/0623, the contents of all of which are incorporated by reference. Modifications thereof which are prepared by introducing thereon a suitable functional group (including an ethyleneically unsaturated polymerizable group) are also examples of block copolymers from which micelles for the disclosed compositions and methods are preferably prepared. Preferable block copolymers are those disclosed in the above-mentioned patents and/or international patent publications. If the block copolymer has a sugar residue on one end of the hydrophilic polymer segment, as in the block copolymer of WO 96/32434, the sugar residue should preferably be subjected to Malaprade oxidation so that a corresponding aldehyde group may be formed.

Lipids are synthetically or naturally-occurring molecules which includes fats, waxes, sterols, prenol lipids, fat-soluble vitamins (such as vitamins A, D, E and K), glycerolipids, monoglycerides, diglycerides, triglycerides, glycerophospholipids, sphingolipids, phospholipids, fatty acids monoglycerides, saccharolipids and others. Lipids can be hydrophobic or amphiphilic small molecules; the amphiphilic nature of some lipids allows them to form structures such as monolayers, vesicles, micelles, liposomes, bi-layers or membranes in an appropriate environment i.e. aqueous environment. Any of a number of lipids can be used as amphiphile molecules, including amphipathic, neutral, cationic, and anionic lipids. Such lipids can be used alone or in combination, and can also include bilayer stabilizing components such as polyamide oligomers (see, e.g., U.S. Pat. No. 6,320,017, “Polyamide Oligomers”, by Ansell), peptides, proteins, detergents, lipid-derivatives, such as PEG coupled to phosphatidylethanolamine and PEG conjugated to ceramides (see, U.S. Pat. No. 5,885,613). In a preferred embodiment, cloaking agents, which reduce elimination of liposomes by the host immune system, can also be included, such as polyamide-oligomer conjugates, e.g., ATTA-lipids, (see, U.S. patent application Ser. No. 08/996,783, filed Feb. 2, 1998) and PEG-lipid conjugates (see, U.S. Pat. Nos. 5,820,873, 5,534,499 and 5,885,613).

Any of a number of neutral lipids can be included, referring to any of a number of lipid species which exist either in an uncharged or neutral zwitterionic form at physiological pH, including diacylphosphatidylcholine, diacylphosphatidylethanolamine, ceramide, sphingomyelin, cephalin, cholesterol, cerebrosides, and diacylglycerols.

Cationic lipids, carry a net positive charge at physiological pH, can readily be used as amphiphile molecules. Such lipids include, but are not limited to, N,N-dioleyl-N,N-dimethylammonium chloride (“DODAC”); N-(2,3-dioleyloxy) propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 3.beta.-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), and N-(1,2-dimyristyloxyprop-3-yl)-N,N-dimethyl-N-hydroxyethyl ammonium bromide (“DMRIE”). Additionally, a number of commercial preparations of cationic lipids can be used, such as LIPOFECTIN (including DOTMA and DOPE, available from GIBCO/BRL), LIPOFECTAMINE (comprising DOSPA and DOPE, available from GIBCO/BRL), and TRANSFECTAM (comprising DOGS, in ethanol, from Promega Corp.).

Anionic lipids can be used as amphiphile molecules and include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

Amphiphatic lipids can also be suitable amphiphile molecules. “Amphipathic lipids” refer to any suitable material, wherein the hydrophobic portion of the lipid material orients into a hydrophobic phase, while the hydrophilic portion orients toward the aqueous phase. Such compounds include, but are not limited to, fatty acids, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleoylphosphatidylcholine. Other phosphorus-lacking compounds, such as sphingolipids, glycosphingolipid families, diacylglycerols, and β-acyloxyacids, can also be used. Additionally, such amphipathic lipids can be readily mixed with other lipids, such as triglycerides and sterols. Zwitterionic lipids are a form of amphiphatic lipid.

Sphingolipids are fatty acids conjugated to the aliphatic amino alcohol sphingosine. The fatty acid can be covalently bond to sphingosine via an amide bond. Any amino acid as described above can be covalently bond to sphingosine to form a sphingolipid. A sphingolipid can be further modified by covalent bonding through the α-hydroxyl group. The modification can include alkyl groups, alkenyl groups, alkynyl groups, aromatic groups, heteroaromatic groups, cyclyl groups, heterocyclyl groups, phosphonic acid groups. Non-limiting examples of shingolipids are N-acylsphingosine, N-Acylsphingomyelin, Forssman antigen.

Saccharolipids are compounds that contain both fatty acids and sugars. The fatty acids are covalently bonded to a sugar backbone. The sugar backbone can contain one or more sugars. The fatty acids can bond to the sugars via either amide or ester bonds. The sugar can be any sugar base. The fatty acid can be any fatty acid as described elsewhere herein. The provided compositions can comprise either natural or synthetic saccharolipids. Non-limiting saccharolipids are UDP-3-O-(β-hydroxymyristoyl)-GlcNAc, lipid IV A, Kdo2-lipid A.

The disclosed compositions and CAR compositions can include one or more cargo molecules. Generally, the disclosed compositions can include a plurality of cargo molecules. The disclosed compositions can include a single type of cargo molecule or a plurality of different types of cargo molecules. Thus, for example, the disclosed compositions can include a plurality of different types of cargo molecules where a plurality of one or more of the different types of cargo molecules can be present.

Cargo molecules can be any compound, molecule, conjugate, composition, etc. that is desired to be delivered using the disclosed compositions. For example, the cargo molecules can be therapeutic agents, detectable agents, or a combination. For example, the cargo molecules can be membrane perturbing molecules, pro-apoptotic molecules, pore-generating molecules, antimicrobial molecules, mitochondria-affecting molecules, mitochondria-targeted molecules, or a combination. Examples of some useful cargo molecules are described below and elsewhere herein.

Cargo molecules can be associated with and arranged in the compositions in a variety of configurations. In some forms, cargo molecules can be associated with, conjugated to, and/or covalently coupled to a plurality of surface molecules. In some forms, cargo molecules can be associated with, conjugated to, and/or covalently coupled to a plurality of homing molecules. In some forms, cargo molecules can be associated with, conjugated to, and/or covalently coupled to a plurality of homing molecules, wherein the homing molecules can be associated with, conjugated to, and/or covalently coupled to a plurality of surface molecules. Combinations of these combinations can also be used.

Membrane perturbing molecules include molecules that can disrupt membranes, that can form pores in membranes, that can make membranes leaky, that can be targeted to or affect intracellular membranes or organelles, such mitochondria or lysosomes. Some forms of membrane perturbing molecules can be pro-apoptotic while others can be non-apoptotic. Some forms of membrane perturbing molecules can be pro-apoptotic for only some types of cells.

In some forms, one or more of the homing molecules can comprise the amino acid sequence CGKRK (SEQ ID NO:1). In some forms, one or more of the membrane perturbing molecules can comprise the amino acid sequence _{D}(KLAKLAK)₂ (SEQ ID NO:3) or a conservative variant thereof, (KLAKLAK)₂ (SEQ ID NO:3) or a conservative variant thereof, (KLAKKLA)₂ (SEQ ID NO:5) or a conservative variant thereof, (KAAKKAA)₂ (SEQ ID NO:6) or a conservative variant thereof, (KLGKKLG)₃ (SEQ ID NO:7) or a conservative variant thereof, or a combination. In some forms, one or more of the membrane perturbing molecules can comprise the amino acid sequence _{D}(KLAKLAK)₂ (SEQ ID NO:3), (KLAKLAK)₂ (SEQ ID NO:3), (KLAKKLA)₂ (SEQ ID NO:5), (KAAKKAA)₂ (SEQ ID NO:6), (KLGKKLG)₃ (SEQ ID NO:7), or a combination. In some forms, one or more of the membrane perturbing molecules can comprise the amino acid sequence _{D}(KLAKLAK)₂ (SEQ ID NO:3) or a conservative variant thereof. In some forms, one or more of the membrane perturbing molecules can comprise the amino acid sequence _{D}(KLAKLAK)₂ (SEQ ID NO:3). Membrane perturbing peptides of this type are described in Ellerby, Nature Medicine 5, 1032-1038 (1999), which is hereby incorporated by reference for its description of such peptides.

A plurality of modified and/or unmodified membrane perturbing molecules can each be independently selected from, for example, an amino acid segment comprising a modified or unmodified form of the amino acid sequence of a homing peptide, an amino acid segment comprising a modified or unmodified form of the amino acid sequence _{D}(KLAKLAK)₂ (SEQ ID NO:3), (KLAKLAK)₂ (SEQ ID NO:3), (KLAKKLA)₂ (SEQ ID NO:5), (KAAKKAA)₂ (SEQ ID NO:6), (KLGKKLG)₃ (SEQ ID NO:7), or a combination. A plurality of the membrane perturbing molecules can each independently comprise an amino acid segment comprising a modified or unmodified form of the amino acid sequence of a homing peptide.

The composition or CAR composition can comprise a sufficient number and composition of membrane perturbing molecules (modified or not) such that the composition has a membrane perturbing effect on the target. In one example, sufficiency of the number and composition of modified and/or unmodified membrane perturbing molecules can be determined by assessing membrane disruption, apoptosis, and/or therapeutic effect on the target.

The composition or CAR composition can comprise any number of modified and/or unmodified membrane perturbing molecules. By way of example, the composition, or CAR composition can comprise at least 1, 5, 10, 15, 20, 25, 50, 75, 100, 125, 150, 175, 200, 225, 250, 275, 300, 325, 350, 375, 400, 425, 450, 475, 500, 525, 550, 575, 600, 625, 650, 675, 700, 625, 750, 775, 800, 825, 850, 875, 900, 925, 950, 975, 1000, 1100, 1200, 1300, 1400, 1500, 1600, 1700, 1800, 1900, 2000, 2250, 2500, 2750, 3000, 3500, 4000, 4500, 5000, 5500, 6000, 6500, 7000, 7500, 8000, 8500, 9000, 9500, 10,000, 15,000, 20,000, 25,000, 30,000, 35,000, 40,000, 45,000, 50,000, 75,000, or 100,000, or more modified and/or unmodified membrane perturbing molecules. The composition can also comprise any number in between those numbers listed above.

Membrane perturbing molecules can be associated with and arranged in the compositions in a variety of configurations. In some forms, membrane perturbing molecules can be associated with, conjugated to, and/or covalently coupled to a plurality of surface molecules. In some forms, membrane perturbing molecules can be associated with, conjugated to, and/or covalently coupled to a plurality of homing molecules. In some forms, membrane perturbing molecules can be associated with, conjugated to, and/or covalently coupled to a plurality of homing molecules, wherein the homing molecules can be associated with, conjugated to, and/or covalently coupled to a plurality of surface molecules. Combinations of these combinations can also be used.

The disclosed membrane perturbing molecules can include modified forms of membrane perturbing molecules. The membrane perturbing molecules can have any useful modification. For example, some modifications can stabilize the membrane perturbing molecule. For example, the disclosed membrane perturbing molecules include methylated membrane perturbing molecules. Methylated membrane perturbing molecules are particularly useful when the membrane perturbing molecule includes a protein, peptide or amino acid segment. For example, a membrane perturbing molecule can be a modified membrane perturbing molecule, where, for example, the modified membrane perturbing molecule includes a modified amino acid segment or amino acid sequence. For example, a modified membrane perturbing molecule can be a methylated membrane perturbing molecule, where, for example, the methylated membrane perturbing molecule includes a methylated amino acid segment or amino acid sequence. Other modifications can be used, either alone or in combination. Where the membrane perturbing molecule is, or includes, a protein, peptide, amino acid segment and/or amino acid sequences, the modification can be to the protein, peptide, amino acid segment, amino acid sequences and/or any amino acids in the protein, peptide, amino acid segment and/or amino acid sequences. Amino acid and peptide modifications are known to those of skill in the art, some of which are described below and elsewhere herein. Methylation is a particularly useful modification for the disclosed membrane perturbing molecules. Using modified forms of membrane perturbing molecules can increase their effectiveness.

The disclosed compositions, surface molecules, cargo molecules, peptides, proteins, amino acid sequences, etc. can comprise one or more internalization elements, tissue penetration elements, or both. Internalization elements and tissue penetration elements can be incorporated into or fused with other peptide components of the composition, such as peptide homing molecules and peptide cargo molecules. Internalization elements are molecules, often peptides or amino acid sequences, that allow the internalization element and components with which it is associated, to pass through biological membranes. Tissue penetration elements are molecules, often peptides or amino acid sequences, that allow the tissue penetration element and components with which it is associated to passage into and through tissue. Some molecules, such as CAR peptides and CendR elements, function as both internalization elements and tissue penetration elements.

Internalization elements include, for example, cell-penetrating peptides (CPPs) and CAR peptides. Peptides that are internalized into cells are commonly referred to as cell-penetrating peptides. There are two main classes of such peptides: hydrophobic and cationic (Zorko and Langel, 2005). The cationic peptides, which are commonly used to introduce nucleic acids, proteins into cells, include the prototypic cell-penetrating peptides (CPP), Tat, and penetratin (Derossi et al., 1998; Meade and Dowdy, 2007). A herpes virus protein, VP22, is capable of both entering and exiting cells and carrying a payload with it (Elliott and O'Hare, 1997; Brewis et al., 2003).

Association of the components of the disclosed compositions can be aided or accomplished via molecules, conjugates and/or compositions. Where such molecules, conjugates and/or compositions are other than CAR peptides, surface molecules, homing molecules, accessory molecules, or cargo molecules (such as membrane perturbing molecules, internalization elements, tissue penetration elements, and moieties), they can be referred to herein as linkers. Such linkers can be any molecule, conjugate, composition, etc. that can be used to associate components of the disclosed compositions. Generally, linkers can be used to associate components other than surface molecules to surface molecules. Useful linkers include materials that are biocompatible, have low bioactivity, have low antigenicity, etc. That is, such useful linker materials can serve the linking/association function without adding unwanted bioreactivity to the disclosed compositions. Many such materials are known and used for similar linking and association functions. Polymer materials are a particularly useful form of linker material. For example, polyethylene glycols can be used.

Linkers are useful for achieving useful numbers and densities of the components (such as homing molecules and membrane perturbing molecules) on surface molecules. For example, linkers of fibrous form are useful for increasing the number of components per surface molecule or per a given area of the surface molecule. Similarly, linkers having a branching form are useful for increasing the number of components per surface molecule or per a given area of the surface molecule. Linkers can also have a branching fibrous form.

Sufficiency of the number and composition of homing molecules in the composition can be determined by assessing homing to the target and effectively delivery of the cargo molecules in a non-human animal. The composition or CAR composition can comprise a sufficient number and composition of homing molecules (modified or not) such that the composition homes to the target and effectively delivers the cargo molecules. In one example, sufficiency of the number and composition of modified and/or unmodified homing molecules can be determined by assessing cargo delivery and/or therapeutic effect on the target. Sufficiency of the number and composition of membrane perturbing molecules can be determined by assessing membrane perturbing effect of the composition in a non-human animal. The composition or CAR composition can comprise a sufficient number and composition of membrane perturbing molecules (modified or not) such that the composition has a membrane perturbing effect on the target. In one example, sufficiency of the number and composition of modified and/or unmodified membrane perturbing molecules can be determined by assessing membrane disruption, apoptosis, and/or therapeutic effect on the target.

The composition or CAR composition can comprise a sufficient density and composition of homing molecules such that the composition homes to the target and effectively delivers the cargo molecules. Sufficiency of the density and composition of homing molecules can be determined by assessing cargo delivery and/or therapeutic effect on the target in a non-human animal. The composition or CAR composition can comprise a sufficient density and composition of membrane perturbing molecules such that the composition has a membrane perturbing effect on the target. Sufficiency of the density and composition of membrane perturbing molecules can be determined by assessing membrane disruption, apoptosis, and/or therapeutic effect on the target in a non-human animal.

The density of homing molecules and/or membrane perturbing molecules on a surface molecule can be described in any suitable manner. For example, the density can be expressed as the number of homing molecules and/or membrane perturbing molecules per, for example, a given area, surface area, volume, unit, subunit, arm, etc. of the surface molecule. The density can also be relative to, for example, the area, surface area, volume, unit, subunit, arm, etc. of the entire surface molecule or to the area, surface area, volume, unit, subunit, arm, etc. of a portion of the surface molecule. For example, a sufficient density of homing molecule and/or membrane perturbing molecule can be present in a portion of the surface molecule. The presence of this dense portion can cause clotting and amplify the accumulation of the composition. Thus, a composition having a sufficient density of homing molecules and/or membrane perturbing molecules can have a threshold density (or above) for the entire surface molecule or for just one or more portions of the surface molecule. Unless otherwise stated, densities refer to average density over the designated portion of the surface molecule. For example, a density of 1 homing molecule per square nM of the surface molecule refers to an average density of the homing molecules over the entire surface molecule. As another example, a density of 1 homing molecule per square nM of a portion of the surface molecule refers to an average density of the homing molecules over just that portion of the surface molecule.

The density can be measured or calculated in any suitable manner. For example, the number or amount of homing molecules and/or membrane perturbing molecules present on a surface molecule or group of surface molecules can be measured by, for example, detecting the level or intensity of signal produced by labeled homing molecules and/or membrane perturbing molecules and calculating the density based on the structural characteristics of the surface molecule.

The density or threshold density of homing molecules and/or membrane perturbing molecules can be, for example, at least 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, or 1000 homing molecules and/or membrane perturbing molecules per square nM of the entire or a portion of the surface molecule. The composition can also comprise any density in between those densities listed above.

The density or threshold density of homing molecules and/or membrane perturbing molecules can be, for example, at least 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5500, 6000, 6500, 7000, 7500, 8000, 8500, 900, 9500, 10,000 homing molecules and/or membrane perturbing molecules per square μM of the entire or a portion of the surface molecule. The composition can also comprise any density in between those densities listed above.

The density or threshold density of homing molecules and/or membrane perturbing molecules can be, for example, at least 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, or 1000 homing molecules and/or membrane perturbing molecules per cubic nM of the entire or a portion of the surface molecule. The composition can also comprise any density in between those densities listed above.

The density or threshold density of homing molecules and/or membrane perturbing molecules can be, for example, at least 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5500, 6000, 6500, 7000, 7500, 8000, 8500, 900, 9500, 10,000 homing molecules and/or membrane perturbing molecules per cubic μM of the entire or a portion of the surface molecule. The composition can also comprise any density in between those densities listed above.

The number of homing molecules and/or membrane perturbing molecules on a surface molecule can be described in any suitable manner. For example, the number can be expressed as the number of homing molecules and/or membrane perturbing molecules per, for example, a given area, surface area, volume, unit, subunit, arm, etc. of the surface molecule. The number can also be relative to, for example, the area, surface area, volume, unit, subunit, arm, etc. of the entire surface molecule or to the area, surface area, volume, unit, subunit, arm, etc. of a portion of the surface molecule. For example, a sufficient number of homing molecule and/or membrane perturbing molecule can be present in a portion of the surface molecule. The presence of this dense portion can cause clotting and amplify the accumulation of the composition. Thus, a composition having a sufficient number of homing molecules and/or membrane perturbing molecules can have a threshold number (or above) for the entire surface molecule or for just one or more portions of the surface molecule.

The number can be measured or calculated in any suitable manner. For example, the number or amount of homing molecules and/or membrane perturbing molecules present on a surface molecule or group of surface molecules can be measured by, for example, detecting the level or intensity of signal produced by labeled homing molecules and/or membrane perturbing molecules and calculating the number based on the structural characteristics of the surface molecule.

The number or threshold number of homing molecules and/or membrane perturbing molecules can be, for example, at least 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5500, 6000, 6500, 7000, 7500, 8000, 8500, 900, 9500, 10,000 homing molecules and/or membrane perturbing molecules on the surface molecule. The composition can also comprise any number in between those numbers listed above.

The number or threshold number of homing molecules and/or membrane perturbing molecules can be, for example, at least 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, or 1000 homing molecules and/or membrane perturbing molecules per square nM of the entire or a portion of the surface molecule. The composition can also comprise any number in between those numbers listed above.

The number or threshold number of homing molecules and/or membrane perturbing molecules can be, for example, at least 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5500, 6000, 6500, 7000, 7500, 8000, 8500, 900, 9500, 10,000 homing molecules and/or membrane perturbing molecules per square μM of the entire or a portion of the surface molecule. The composition can also comprise any number in between those numbers listed above.

The number or threshold number of homing molecules and/or membrane perturbing molecules can be, for example, at least 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, or 1000 homing molecules and/or membrane perturbing molecules per cubic nM of the entire or a portion of the surface molecule. The composition can also comprise any number in between those numbers listed above.

The number or threshold number of homing molecules and/or membrane perturbing molecules can be, for example, at least 1, 2, 3, 4, 5, 6, 7, 8, 9, 10, 12, 14, 16, 18, 20, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, 380, 400, 420, 440, 460, 480, 500, 550, 600, 650, 700, 750, 800, 850, 900, 950, 1000, 1100, 1200, 1300, 1400, 1500, 1600, 1700, 1800, 1900, 2000, 2200, 2400, 2600, 2800, 3000, 3200, 3400, 3600, 3800, 4000, 4200, 4400, 4600, 4800, 5000, 5500, 6000, 6500, 7000, 7500, 8000, 8500, 900, 9500, 10,000 homing molecules and/or membrane perturbing molecules per cubic μM of the entire or a portion of the surface molecule. The composition can also comprise any number in between those numbers listed above.

Homing molecules can be used that are clot-binding compounds that recognize clotted plasma proteins and selectively homes to tumors, where it binds to vessel walls and tumor stroma. Surface molecules coupled with the clot-binding compounds can accumulate in tumor vessels or at wound sites, where they induce additional local clotting, thereby producing new binding sites for more particles. The system mimics platelets, which also circulate freely but accumulate at a diseased site and amplify their own accumulation at that site.

Disclosed are linkers for associating components of the disclosed compositions. Such linkers can be any molecule, conjugate, composition, etc. that can be used to associate components of the disclosed compositions. Generally, linkers can be used to associate components other than surface molecules to surface molecules. Useful linkers include materials that are biocompatible, have low bioactivity, have low antigenicity, etc. That is, such useful linker materials can serve the linking/association function without adding unwanted bioreactivity to the disclosed compositions. Many such materials are known and used for similar linking and association functions. Polymer materials are a particularly useful form of linker material. For example, polyethylene glycols can be used.

Linkers are useful for achieving useful numbers and densities of the components (such as homing molecules and membrane perturbing molecules) on surface molecules. For example, linkers of fibrous form are useful for increasing the number of components per surface molecule or per a given area of the surface molecule. Similarly, linkers having a branching form are useful for increasing the number of components per surface molecule or per a given area of the surface molecule. Linkers can also have a branching fibrous form.

Linkers of different lengths can be used to bind the disclosed components to surface molecules and to each other. A flexible linker can function well even if relatively short, while a stiffer linker can be longer to allow effective exposure and density. The length of a linker can refer to the number of atoms in a continuous covalent chain between the attachment points on the components being linked or to the length (in nanometers, for example) of a continuous covalent chain between the attachment points on the components being linked. Unless the context clearly indicates otherwise, the length refers to the shortest continuous covalent chain between the attachment points on the components being linked not accounting for side chains, branches, or loops. Due to flexibility of the linker, all of the linkers may not have same distance from the surface molecule. Thus linkers with different chain lengths can make the resulting composition more effective (by increasing density, for example). Branched linkers bearing multiple components also allow attachment of more than one component at a given site of the surface molecule. Useful lengths for linkers include at least, up to, about, exactly, or between 10, 15, 20, 25, 30, 35, 40, 45, 50, 60, 70, 80, 90, 100, 120, 140, 150, 160, 180, 200, 250, 300, 350, 400, 450, 500, 550, 600, 650, 700, 750, 800, 850, 900, 950, 1,000, 2,000, 3,000, 4,000, 5,000, 6,000, 7,000, 8,000, 9,000, and 10,000 atoms. Useful lengths for linkers include at least, up to, about, exactly, or between 10, 15, 20, 25, 30, 35, 40, 45, 50, 60, 70, 80, 90, 100, 120, 140, 150, 160, 180, 200, 250, 300, 350, 400, 450, 500, 550, 600, 650, 700, 750, 800, 850, 900, 950, 1,000, 2,000, 3,000, 4,000, 5,000, 6,000, 7,000, 8,000, 9,000, and 10,000 nanometers. Any range of these lengths and all lengths between the listed lengths are specifically contemplated.

Hydrophilic or water-solubility linkers can increase the mobility of the attached components. Examples of water-soluble, biocompatible polymers which can serve as linkers include, but are not limited to polymers such polyethylene glycol (PEG), polyethylene oxide (PEO), polyvinyl alcohol, polyhydroxyethyl methacrylate, polyacrylamide, and natural polymers such as hyaluronic acid, chondroitin sulfate, carboxymethylcellulose, and starch. Useful forms of branched tethers include star PEO and comb PEO. Star PEO can be formed of many PEO “arms” emanating from a common core.

Polyethylene glycols (PEGs) are simple, neutral polyethers which have been given much attention in biotechnical and biomedical applications (Milton Harris, J. (ed) “Poly(ethylene glycol) chemistry, biotechnical and biomedical applications” Plenum Press, New York, 1992). PEGs are soluble in most solvents, including water, and are highly hydrated in aqueous environments, with two or three water molecules bound to each ethylene glycol segment; this hydration phenomenon has the effect of preventing adsorption either of other polymers or of proteins onto PEG-modified surfaces. Furthermore, PEGs may readily be modified and bound to other molecules with only little effect on their chemistry. Their advantageous solubility and biological properties are apparent from the many possible uses of PEGs and copolymers thereof, including block copolymers such as PEG-polyurethanes and PEG-polypropylenes. Appropriate molecular weights for PEG linkers used in the disclosed compositions can be from about 120 daltons to about 20 kilodaltons. For example, PEGs can be at least, up to, about, exactly, or between 100, 150, 200, 250, 300, 350, 400, 450, 500, 600, 700, 800, 900, 1000, 1200, 1400, 1500, 1600, 1800, 2000, 2500, 3000, 3500, 4000, 4500, 5000, 5500, 6000, 6500, 7000, 7500, 8000, 8500, 9000, 9500, 10,000, 20,000, 30,000, 40,000, and 50,000 daltons. Any range of these masses and all masses between the listed masses are specifically contemplated. PEGs are usually available as mixtures of somewhat heterogeneous masses with a stated average mass (PEG-5000, for example).

The disclosed compositions can be produced using any suitable techniques. Many techniques, reactive groups, chemistries, etc. for linking components of the types disclosed herein are known and can be used with the disclosed components and compositions.

Protein crosslinkers that can be used to crosslink other molecules, elements, moieties, etc. to the disclosed compositions, surface molecules, homing molecules, membrane perturbing molecules, internalization elements, tissue penetration elements, CAR peptides, compositions, peptides, amino acid sequences, etc. are known in the art and are defined based on utility and structure and include DSS (Disuccinimidylsuberate), DSP (Dithiobis(succinimidylpropionate)), DTSSP (3,3′-Dithiobis(sulfosuccinimidylpropionate)), SULFO BSOCOES (Bis[2-(sulfosuccinimdooxycarbonyloxy) ethyl]sulfone), BSOCOES (Bis[2-(succinimdooxycarbonyloxy)ethyl]sulfone), SULFO DST (Disulfosuccinimdyltartrate), DST (Disuccinimdyltartrate), SULFO EGS (Ethylene glycolbis(succinimidylsuccinate)), EGS (Ethylene glycolbis(sulfosuccinimidylsuccinate)), DPDPB (1,2-Di[3′-(2′-pyridyldithio) propionamido]butane), BSSS (Bis(sulfosuccinimdyl) suberate), SMPB (Succinimdyl-4-(p-maleimidophenyl) butyrate), SULFO SMPB (Sulfosuccinimdyl-4-(p-maleimidophenyl) butyrate), MBS (3-Maleimidobenzoyl-N-hydroxysuccinimide ester), SULFO MBS (3-Maleimidobenzoyl-N-hydroxysulfosuccinimide ester), SIAB (N-Succinimidyl(4-iodoacetyl)aminobenzoate), SULFO SIAB (N-Sulfosuccinimidyl(4-iodoacetyl)aminobenzoate), SMCC (Succinimidyl-4-(N-maleimidomethyl)cyclohexane-1-carboxylate), SULFO SMCC (Sulfosuccinimidyl-4-(N-maleimidomethyl)cyclohexane-1-carboxylate), NHS LC SPDP (Succinimidyl-6-[3-(2-pyridyldithio) propionamido) hexanoate), SULFO NHS LC SPDP (Sulfosuccinimidyl-6-[3-(2-pyridyldithio) propionamido) hexanoate), SPDP (N-Succinimdyl-3-(2-pyridyldithio) propionate), NHS BROMOACETATE (N-Hydroxysuccinimidylbromoacetate), NHS IODOACETATE (N-Hydroxysuccinimidyliodoacetate), MPBH (4-(N-Maleimidophenyl) butyric acid hydrazide hydrochloride), MCCH (4-(N-Maleimidomethyl)cyclohexane-1-carboxylic acid hydrazide hydrochloride), MBH (m-Maleimidobenzoic acid hydrazidehydrochloride), SULFO EMCS(N-(epsilon-Maleimidocaproyloxy) sulfosuccinimide), EMCS(N-(epsilon-Maleimidocaproyloxy) succinimide), PMPI (N-(p-Maleimidophenyl) isocyanate), KMUH (N-(kappa-Maleimidoundecanoic acid) hydrazide), LC SMCC (Succinimidyl-4-(N-maleimidomethyl)-cyclohexane-1-carboxy(6-amidocaproate)), SULFO GMBS (N-(gamma-Maleimidobutryloxy) sulfosuccinimide ester), SMPH (Succinimidyl-6-(beta-maleimidopropionamidohexanoate)), SULFO KMUS (N-(kappa-Maleimidoundecanoyloxy)sulfosuccinimide ester), GMBS (N-(gamma-Maleimidobutyrloxy) succinimide), DMP (Dimethylpimelimidate hydrochloride), DMS (Dimethylsuberimidate hydrochloride), MHBH (Wood's Reagent; Methyl-p-hydroxybenzimidate hydrochloride, 98%), DMA (Dimethyladipimidate hydrochloride).

Components of the disclosed compositions, such as surface molecules, homing molecules, membrane perturbing molecules, internalization elements, tissue penetration elements, etc., can also be coupled using, for example, maleimide coupling. By way of illustration, components can be coupled to lipids by coupling to, for example, 1,2-distearoyl-sn-glycero-3-phosphoethanolamine-N-[maleimide(polyethylene glycol)₂₀₀₀; DSPE-PEG₂₀₀₀-maleimide] (Avanti Polar Lipids) by making use of a free cysteine sulfhydryl group on the component. The reaction can be performed, for example, in aqueous solution at room temperature for 4 hours. This coupling chemistry can be used to couple components of compositions.

Components of the disclosed compositions, such as surface molecules, homing molecules, membrane perturbing molecules, internalization elements, tissue penetration elements, etc., can also be coupled using, for example, amino group-functionalized dextran chemistry. Particles, such as, for example, nanoparticles, nanoworms, and micelles, can be coated with amino group functionalized dextran. Attachment of PEG to aminated particles increases the circulation time, presumably by reducing the binding of plasma proteins involved in opsonization (Moghimi et al., Pharm. Rev. 53, 283-318 (2001)). The particles can have surface modifications, for example, for reticuloendothelial system avoidance (PEG) and homing (homing molecules), endosome escape (pH-sensitive peptide; for example, Pirollo et al., Cancer Res. 67, 2938-43 (2007)), a detectable agent, a therapeutic compound, or a combination. To accommodate all these functions on one particle, optimization studies can be conducted to determine what proportion of the available linking sites at the surface of the particles any one of these elements should occupy to give the best combination of targeting and payload delivery. The cell internalization and/or tissue penetration of such compositions can be mediated by the disclosed CAR peptides, amino acid sequences, proteins, molecules, conjugates, and compositions.

The provided peptides and polypeptides can have additional N-terminal, C-terminal, or intermediate amino acid sequences, e.g., amino acid linkers or tags. The term “amino acid linker” refers to an amino acid sequences or insertions that can be used to connect or separate two distinct peptides, polypeptides, or polypeptide fragments, where the linker does not otherwise contribute to the essential function of the composition. The term “amino acid tag” refers to a distinct amino acid sequence that can be used to detect or purify the provided polypeptide, wherein the tag does not otherwise contribute to the essential function of the composition. The provided peptides and polypeptides can further have deleted N-terminal, C-terminal or intermediate amino acids that do not contribute to the essential activity of the peptides and polypeptides.

Components can be directly or indirectly covalently bound to surface molecules or each other by any functional group (e.g., amine, carbonyl, carboxyl, aldehyde, alcohol). For example, one or more amine, alcohol or thiol groups on the components can be reacted directly with isothiocyanate, acyl azide, N-hydroxysuccinimide ester, aldehyde, epoxide, anhydride, lactone, or other functional groups incorporated onto the surface molecules or other components. Schiff bases formed between the amine groups on the components and aldehyde groups on the surface molecule or other components can be reduced with agents such as sodium cyanoborohydride to form hydrolytically stable amine links (Ferreira et al., J. Molecular Catalysis B: Enzymatic 2003, 21, 189-199). Components can be coupled to surface molecules and other components by, for example, the use of a heterobifunctional silane linker reagent, or by other reactions that activate functional groups on either the surface molecule or the components.

Useful modes for linking components to surface molecules and to other components include heterobifunctional linkers or spacers. Such linkers can have both terminal amine and thiol reactive functional groups for reacting amines on components with sulfhydryl groups, thereby coupling the components in an oriented way. These linkers can contain a variable number of atoms. Examples of such linkers include, but are not limited to, N-Succinimidyl 3-(2-pyridyldithio)propionate (SPDP, 3- and 7-atom spacer), long-chain-SPDP (12-atom spacer), (Succinimidyloxycarbonyl-a-methyl-2-(2-pyridyldithio) toluene) (SMPT, 8-atom spacer), Succinimidyl-4-(N-maleimidomethyl)cyclohexane-1-carboxylate) (SMCC, 11-atom spacer) and Sulfosuccinimidyl-4-(N-maleimidomethyl)cyclohexane-1-carboxylate, (sulfo-SMCC, 11-atom spacer), m-Maleimidobenzoyl-N hydroxysuccinimide ester (MBS, 9-atom spacer), N-(g-maleimidobutyryloxy)succinimide ester (GMBS, 8-atom spacer), N-(g-maleimidobutyryloxy) sulfosuccinimide ester (sulfo-GMBS, 8-atom spacer), Succinimidyl 6-((iodoacetyl)amino) hexanoate (SIAX, 9-atom spacer), Succinimidyl 6-(6-(((4-iodoacetyl)amino)hexanoyl)amino)hexanoate (SIAXX, 16-atom spacer), and p-nitrophenyl iodoacetate (NPIA, 2-atom spacer). One ordinarily skilled in the art also will recognize that a number of other coupling agents or links, with different number of atoms, may be used.

Hydrophilic spacer atoms can be incorporated into linkers to increase the distance between the reactive functional groups. For example, polyethylene glycol (PEG) can be incorporated into sulfo-GMBS. Hydrophilic molecules such as PEG have also been shown to decrease non-specific binding (NSB) and increase hydrophilicity of surfaces when covalently coupled. PEG can also be used as the primary linker material.

Free amine groups of components can also be attached to surface molecules or other components containing reactive amine groups via homobifunctional linkers. Linkers such as dithiobis(succinimidylpropionate) (DSP, 8-atom spacer), disuccinimidyl suberate (DSS, 8-atom spacer), glutaraldehyde (4-atom spacer), Bis[2-(succinimidyloxycarbonyloxy)ethyl]sulfone (BSOCOES, 9-atom spacer), all requiring high pH, can be used for this purpose. Examples of homobifunctional sulfhydryl-reactive linkers include, but are not limited to, 1,4-Di-[3′-2′-pyridyldithio)propion-amido]butane (DPDPB, 16-atom spacer) and Bismaleimidohexane (BMH, 14-atom spacer). For example, these homobifunctional linkers are first reacted with a thiolated surface in aqueous solution (for example PBS, pH 7.4), and then in a second step, the thiolated antibody or protein is joined by the link. Homo- and heteromultifunctional linkers can also be used.

Direct binding of components to thiol, amine, or carboxylic acid functional groups on surface molecules and other components be used to produce compositions which exhibit viral binding (due to increased density of components, for example), resulting in enhanced sensitivity.

As an example, when necessary to achieve high peptide coupling density, additional amino groups can be added to the surface molecules (such as commercially obtained SPIO) as follows: First, to crosslink the particles before the amination step, 3 ml of the colloid (˜10 mgFe/ml in double-distilled water) was added to 5 ml of 5M NaOH and 2 ml of epichlorohydrin (Sigma, St. Louis, Mo.). The mixture was agitated for 24 hours at room temperature to promote interaction between the organic phase (epichlorohydrin) and aqueous phase (dextran-coated particle colloid). In order to remove excess epichlorohydrin, the reacted mixture was dialyzed against double-distilled water for 24 hours using a dialysis cassette (10,000 Da cutoff, Pierce, Rockford Ill.). Amino groups were added to the surface of the particles as follows: 0.02 ml of concentrated ammonium hydroxide (30%) was added to 1 ml of colloid (˜10 mg Fe/ml). The mixture was agitated at room temperature for 24 hours. The reacted mixture was dialyzed against double-distilled water for 24 hours. To further rinse the particles, the colloid was trapped on a MACS®Midi magnetic separation column (Miltenyi Biotec, Auburn Calif.), rinsed with PBS three times, and eluted from the column with 1 ml PBS.

To conjugate CAR peptide (and other peptides) to SPIO, the particles can be re-suspended at a concentration of 1 mg Fe/ml, and heterobifunctional linker N-[a-maleimidoacetoxy]succinimide ester (AMAS; Pierce) was added (2.5 mg linker per 2 mg Fe) under vortexing. After incubation at room temperature for 40 min, the particles were washed 3 times with 10 ml PBS on a MACS column. The peptide with free terminal cysteine was then added (100 μg peptide per 2 mg Fe). After incubation overnight at 4° C. the particles were washed again and re-suspended in PBS at a concentration of 0.35 mg/ml of Fe). To quantify the number of peptide molecules conjugated to the particles, a known amount of stock or AMAS-activated particles was incubated with varying amounts of the peptide. After completion of the incubation the particles were pelleted at 100.000 G using Beckman TLA 100.3 ultracentrifuge rotor (30 min) and the amount of the unbound peptide was quantified by fluorescence. To cleave the conjugated peptide from the particles, the particles were incubated at 37° C. overnight at pH 10. The concentration of free peptide in the supernatant was determined by reading fluorescence and by using the calibration curve obtained for the same peptide. The fluorescence intensity of known amounts of particles was plotted as a function of peptide conjugation density, and the slope equation was used to determine conjugation density in different batches.

Many homing molecules and homing peptides home to the vasculature of the target tissue. However, for the sake of convenience homing is referred to in some places herein as homing to the tissue associated with the vasculature to which the homing molecule or homing peptide may actually home. Thus, for example, a homing peptide that homes to wound vasculature can be referred to herein as homing to wounded tissue or to wounded cells. By including or associating a homing molecule or homing peptide with, for example, a protein, peptide, amino acid sequence, or CAR peptide the protein, peptide, amino acid sequence, or CAR peptide can be targeted or can home to the target of the homing molecule or homing peptide. In this way, the protein, peptide, amino acid sequence, or CAR peptide can be said to home to the target of the homing molecule or homing peptide. For convenience and unless otherwise indicated, reference to homing of a protein, peptide, amino acid sequence, CAR peptide, etc. is intended to indicate that the protein, peptide, amino acid sequence, CAR peptide, etc. includes or is associated with an appropriate homing molecule or homing peptide.

The homing molecule can selectively home to wounded tissue, regenerating tissue, sites of injury, surgical sites, sites of angiogenesis, sites of inflammation, sites of arthritis, lung tissue, pulmonary arterial hypertension lung vasculature, pulmonary arterial hypertension lesions, remodeled pulmonary arteries, or interstitial space of lungs. The CAR composition can selectively home to wounded tissue, regenerating tissue, sites of injury, surgical sites, sites of angiogenesis, sites of inflammation, sites of arthritis, lung tissue, pulmonary arterial hypertension lung vasculature, pulmonary arterial hypertension lesions, remodeled pulmonary arteries, or interstitial space of lungs. The CAR peptide can selectively home to wounded tissue, regenerating tissue, sites of injury, surgical sites, sites of angiogenesis, sites of inflammation, sites of arthritis, lung tissue, pulmonary arterial hypertension lung vasculature, pulmonary arterial hypertension lesions, remodeled pulmonary arteries, or interstitial space of lungs. The cargo molecule can selectively home to wounded tissue, regenerating tissue, sites of injury, surgical sites, sites of angiogenesis, sites of inflammation, sites of arthritis, lung tissue, pulmonary arterial hypertension lung vasculature, pulmonary arterial hypertension lesions, remodeled pulmonary arteries, or interstitial space of lungs. The surface molecule can selectively home to wounded tissue, regenerating tissue, sites of injury, surgical sites, sites of angiogenesis, sites of inflammation, sites of arthritis, lung tissue, pulmonary arterial hypertension lung vasculature, pulmonary arterial hypertension lesions, remodeled pulmonary arteries, or interstitial space of lungs. The membrane perturbing molecule can selectively home to wounded tissue, regenerating tissue, sites of injury, surgical sites, sites of angiogenesis, sites of inflammation, sites of arthritis, lung tissue, pulmonary arterial hypertension lung vasculature, pulmonary arterial hypertension lesions, remodeled pulmonary arteries, or interstitial space of lungs.

The disclosed amino acid sequences, compositions, proteins or peptides can, for example, home to brain cells, brain stem cells, brain tissue, and/or brain vasculature, kidney cells, kidney stem cells, kidney tissue, and/or kidney vasculature, skin cells, skin stem cells, skin tissue, and/or skin vasculature, lung cells, lung tissue, and/or lung vasculature, pancreatic cells, pancreatic tissue, and/or pancreatic vasculature, intestinal cells, intestinal tissue, and/or intestinal vasculature, adrenal gland cells, adrenal tissue, and/or adrenal vasculature, retinal cells, retinal tissue, and/or retinal vasculature, liver cells, liver tissue, and/or liver vasculature, prostate cells, prostate tissue, and/or prostate vasculature, endometriosis cells, endometriosis tissue, and/or endometriosis vasculature, ovary cells, ovary tissue, and/or ovary vasculature, bone cells, bone tissue, and/or bone vasculature, bone marrow cells, bone marrow tissue, and/or bone marrow vasculature, cartilage cells, cartilage tissue, and/or cartilage vasculature, stem cells, embryonic stem cells, pluripotent stem cells, induced pluripotent stem cells, adult stem cells, hematopoietic stem cells, neural stem cells, mesenchymal stem cells, mammary stem cells, endothelial stem cells, olfactory adult stem cells, neural crest stem cells, cancer stem cells, blood cells, erythrocytes, platelets, leukocytes, granulocytes, neutrophils, eosinphils, basophils, lymphoid cells, lymphocytes, monocytes, wound vasculature, vasculature of injured tissue, vasculature of inflamed tissue, atherosclerotic plaques, or a combination.

Examples of homing molecules and homing peptides are known. Examples include: Brain homing peptides such as: CNSRLHLRC (SEQ ID NO:114), CENWWGDVC (SEQ ID NO:115), WRCVLREGPAGGCAWFNRHRL (SEQ ID NO:116), CLSSRLDAC (SEQ ID NO:117), CVLRGGRC (SEQ ID NO:118), CNSRLQLRC (SEQ ID NO:119), CGVRLGC (SEQ ID NO:120), CKDWGRIC (SEQ ID NO:121), CLDWGRIC (SEQ ID NO:122), CTRITESC (SEQ ID NO:123), CETLPAC (SEQ ID NO:124), CRTGTLFC (SEQ ID NO:125), CGRSLDAC (SEQ ID NO:126), CRHWFDVVC (SEQ ID NO:127), CANAQSHC (SEQ ID NO:128), CGNPSYRC (SEQ ID NO:129), YPCGGEAVAGVSSVRTMCSE (SEQ ID NO:130), LNCDYQGTNPATSVSVPCTV (SEQ ID NO:131); kidney homing peptides such as: CLPVASC (SEQ ID NO:132), CGAREMC (SEQ ID NO:133), CKGRSSAC (SEQ ID NO:134), CWARAQGC (SEQ ID NO:135), CLGRSSVC (SEQ ID NO:136), CTSPGGSC (SEQ ID NO:137), CMGRWRLC (SEQ ID NO:138), CVGECGGC (SEQ ID NO:139), CVAWLNC (SEQ ID NO:140), CRRFQDC (SEQ ID NO:141), CLMGVHC (SEQ ID NO:142), CKLLSGVC (SEQ ID NO:143), CFVGHDLC (SEQ ID NO:144), CRCLNVC (SEQ ID NO:145), CKLMGEC (SEQ ID NO:146); skin homing peptides such as: CARSKNKDC (SEQ ID NO:10), CRKDKC (SEQ ID NO:2), CVALCREACGEGC (SEQ ID NO:149), CSSGCSKNCLEMC (SEQ ID NO:150), CIGEVEVC (SEQ ID NO:151), CKWSRLHSC (SEQ ID NO:152), CWRGDRKIC (SEQ ID NO:153), CERVVGSSC (SEQ ID NO:154), CLAKENVVC (SEQ ID NO:155); lung homing peptides such as: CGFECVRQCPERC (SEQ ID NO:156), CGFELETC (SEQ ID NO:157), CTLRDRNC (SEQ ID NO:158), CIGEVEVC (SEQ ID NO:151), CGKRYRNC (SEQ ID NO:161), CLRPYLNC (SEQ ID NO:162), CTVNEAYKTRMC (SEQ ID NO:163), CRLRSYGTLSLC (SEQ ID NO:164), CRPWHNQAHTEC (SEQ ID NO:165); pancreas homing peptides such as: SWCEPGWCR (SEQ ID NO:166), CKAAKNK (SEQ ID NO:167), CKGAKAR (SEQ ID NO:168), VGVGEWSV (SEQ ID NO:92); intestine homing peptides such as: YSGKWGW (SEQ ID NO:93); uterus homing peptides such as: GLSGGRS (SEQ ID NO:94); adrenal gland homing peptides such as: LMLPRAD (SEQ ID NO:95), LPRYLLS (SEQ ID NO:96); retina homing peptides such as: CSCFRDVCC (SEQ ID NO:97), CRDVVSVIC (SEQ ID NO:98); gut homing peptides such as: YSGKWGK (SEQ ID NO:99), GISALVLS (SEQ ID NO:100), SRRQPLS (SEQ ID NO:101), MSPQLAT (SEQ ID NO:102), MRRDEQR (SEQ ID NO:103), QVRRVPE (SEQ ID NO:104), VRRGSPQ (SEQ ID NO:105), GGRGSWE (SEQ ID NO:106), FRVRGSP (SEQ ID NO:25), RVRGPER (SEQ ID NO:26); liver homing peptides such as: VKSVCRT (SEQ ID NO:27), WRQNMPL (SEQ ID NO:28), SRRFVGG (SEQ ID NO:29), ALERRSL (SEQ ID NO:30), ARRGWTL (SEQ ID NO:31); prostate homing peptides such as: SMSIARL (SEQ ID NO:32), VSFLEYR (SEQ ID NO:33), RGRWLAL (SEQ ID NO:34); ovary homing peptides such as: EVRSRLS (SEQ ID NO:35), VRARLMS (SEQ ID NO:36), RVGLVAR (SEQ ID NO:37), RVRLVNL (SEQ ID NO:38); Clot binding homing peptide such as: CREKA (SEQ ID NO:12), CLOT1, and CLOT2; heart homing peptides such as: CRPPR (SEQ ID NO:39), CGRKSKTVC (SEQ ID NO:40), CARPAR (SEQ ID NO:41), CPKRPR (SEQ ID NO:42), CKRAVR (SEQ ID NO:43), CRNSWKPNC (SEQ ID NO:44), RGSSS (SEQ ID NO:19), CRSTRANPC (SEQ ID NO:16), CPKTRRVPC (SEQ ID NO:17), CSGMARTKC (SEQ ID NO:45), GGGVFWQ (SEQ ID NO:61), HGRVRPH (SEQ ID NO:107), VVLVTSS (SEQ ID NO:148), CLHRGNSC (SEQ ID NO:159), CRSWNKADNRSC (SEQ ID NO:160), CGRKSKTVC (SEQ ID NO:40), CKRAVR (SEQ ID NO:43), CRNSWKPNC (SEQ ID NO:44), CPKTRRVPC (SEQ ID NO:17), CSGMARTKC (SEQ ID NO:45), CARPAR (SEQ ID NO:107), CPKRPR (SEQ ID NO:42); tumor blood vessel homing peptide such as: CNGRC (SEQ ID NO:68) and other peptides with the NGR motif (U.S. Pat. Nos. 6,177,542 and 6,576,239; U.S. Patent Application Publication No. 20090257951); RGD peptides, and RGR peptides. Other homing peptides include CSRPRRSEC (SEQ ID NO:108), CSRPRRSVC (SEQ ID NO:109) and CSRPRRSWC (SEQ ID NO:110) (Hoffman et al., Cancer Cell, vol. 4 (2003)), F3 (KDEPQRRSARLSAKPAPPKPEPKPKKAPAKK; (SEQ ID NO:111)), PQRRSARLSA (SEQ ID NO:112), PKRRSARLSA (SEQ ID NO:113) (U.S. Pat. No. 7,544,767), and CGRECPRLCQSSC (SEQ ID NO:62), which home to tumors.

Homing molecules can also be defined by their targets. For example, numerous antigens and proteins are known that can be useful for targeting. Any molecule that can bind, selectively bind, home, selectively, target, selectively target, etc. such target molecules can be used as a homing molecule. For example, antibodies, nucleic acid aptamers, and compounds that can bind to target molecules can be used as homing molecules. Examples of useful target molecules for homing molecules include αv integrins, αvβ3 integrin, αvβ5 integrin, α5β1 integrin, aminopeptidase N, tumor endothelial markers (TEMs), endosialin, p32, gC1q receptor, annexin-1, nucleolin, fibronectin ED-B, fibrin-fibronectin complexes, interleukin-11 receptor α, and protease-cleaved collagen IV. These and other examples are described and referred to in Ruoslahti et al., J. Cell Biology, 2010 (doi: 10.1083/jbc.200910104), which is hereby incorporated by reference in its entirety and specifically for its description of and references to target molecules.

The composition or CAR composition, can comprise any number of homing molecules. By way of example, the composition or CAR composition, can comprise at least 1, 5, 10, 15, 20, 25, 50, 75, 100, 125, 150, 175, 200, 225, 250, 275, 300, 325, 350, 375, 400, 425, 450, 475, 500, 525, 550, 575, 600, 625, 650, 675, 700, 625, 750, 775, 800, 825, 850, 875, 900, 925, 950, 975, 1000, 1100, 1200, 1300, 1400, 1500, 1600, 1700, 1800, 1900, 2000, 2250, 2500, 2750, 3000, 3500, 4000, 4500, 5000, 5500, 6000, 6500, 7000, 7500, 8000, 8500, 9000, 9500, 10,000, 15,000, 20,000, 25,000, 30,000, 35,000, 40,000, 45,000, 50,000, 75,000, or 100,000, or more homing molecules. The composition or CAR composition, can also comprise any number in between those numbers listed above.

It is understood that, although many homing and targeting motifs and sequences are shown with cysteine residues at one or both ends, such cysteine residues are generally not required for homing function. Generally, such cysteines are present due to the methods by which the homing and targeting sequences were identified. Such terminal cysteines can be used to, for example, circularize peptides, such as those disclosed herein. For these reasons, it is also understood that cysteine residues can be added to the ends of any of the disclosed peptides.

Useful NGR peptides include peptide such as X₂CNGRCX₂ (SEQ ID NO:89), CX₂(C/X)NGR(C/X)X₂C (SEQ ID NO:90), and CNGRCX₆ (SEQ ID NO:91) (where “X” is any amino acid), which can be linear or circular. Examples of NGR peptides include CNGRCVSGCAGRC (SEQ ID NO:63), NGRAHA (SEQ ID NO:24), CVLNGRMEC (SEQ ID NO:67), CNGRC (SEQ ID NO:68), ALNGREESP (SEQ ID NO:66), CVLNGRME (SEQ ID NO:87), CKVCNGRCCG (SEQ ID NO:88), CEMCNGRCMG (SEQ ID NO:69), CPLCNGRCAL (SEQ ID NO:70), CPTCNGRCVR (SEQ ID NO:71), CGVCNGRCGL (SEQ ID NO:72), CEQCNGRCGQ (SEQ ID NO:73), CRNCNGRCEG (SEQ ID NO:74), CVLCNGRCWS (SEQ ID NO:75), CVTCNGRCRV (SEQ ID NO:76), CTECNGRCQL (SEQ ID NO:77), CRTCNGRCLE (SEQ ID NO:78), CETCNGRCVG (SEQ ID NO:79), CAVCNGRCGF (SEQ ID NO:80), CRDLNGRKVM (SEQ ID NO:81), CSCCNGRCGD (SEQ ID NO:82), CWGCNGRCRM (SEQ ID NO:83), CPLCNGRCAR (SEQ ID NO:84), CKSCNGRCLA (SEQ ID NO:85), CVPCNGRCHE (SEQ ID NO:86), CQSCNGRCVR (SEQ ID NO:47), CRTCNGRCQV (SEQ ID NO:48), CVQCNGRCAL (SEQ ID NO:49), CRCCNGRCSP (SEQ ID NO:50), CASNNGRVVL (SEQ ID NO:51), CGRCNGRCLL (SEQ ID NO:52), CWLCNGRCGR (SEQ ID NO:53), CSKCNGRCGH (SEQ ID NO:54), CVWCNGRCGL (SEQ ID NO:55), CIRCNGRCSV (SEQ ID NO:56), CGECNGRCVE (SEQ ID NO:57), CEGVNGRRLR (SEQ ID NO:58), CLSCNGRCPS (SEQ ID NO:59), CEVCNGRCAL (SEQ ID NO:60).

Useful peptides for tumor targeting include, for example, iRGD, CAR, LyP-1, iNGR, and RGR peptides. The prototypic tumor-homing CendR peptide, iRGD, which was used in generating the results described herein. CAR has tumor-penetrating properties. This peptide has a unique target within tumors; it preferentially accumulates in the hypoxic/low nutrient areas of tumors (Laakkonen et al., 2002; 2004; Karmali et al., 2009). CRGRRST (RGR; Joyce et al., 2003) is a peptide that has been successfully used in targeting a cytokine antibody combination into tumors (Hamzah et al., 2008). This peptide is linear, which simplifies the synthesis. NGR peptides home to angiogenic vasculature, including angiogenic vasculature associated with tumors, and αᵥ integrin and α₅β₁ integrin (U.S. Pat. Nos. 6,576,239 and 6,177,542 and U.S. Patent Application Publication No. 20090257951). RGR is at least to some extent tumor type-specific (Joyce et al., 2003), but the tumor types recognized by the two peptides seem to be partially different, which may be an advantage in testing combinations with the pan-tumor iRGD.

RGD peptides are peptides that contain the RGD (Arg-Gly-Asp) motif and that home to angiogenesis and tumor vasculature. NGR peptides are peptides that contain the NGR (Asn-Gly-Arg) motif and that home to angiogenesis and tumor vasculature. Examples of NGR peptides include CNGRCVSGCAGRC (SEQ ID NO:63), NGRAHA (SEQ ID NO:24), CVLNGRMEC (SEQ ID NO:67), and CNGRC (SEQ ID NO:68). GSL peptides are peptides that contain the GSL (Gly-Ser-Leu) motif and that home to tumor vasculature. Examples of a GSL peptide include CGSLVRC (SEQ ID NO:65) and CLSGSLSC (SEQ ID NO:64).

Internalizing RGD (iRGD) refers to peptides that combine an RGD motif and a CendR element. For example, cyclic RGD peptide having the sequence CRGDK/RGPD/EC (SEQ ID NO:11) is exceptionally effective in orchestrating extravasation and spreading of linked payloads within tumor tissue, and subsequently internalizing within tumor cells. The iRGD peptide incorporates two functional elements: the RGD motif that gives tumor specificity (Pierschbacher and Ruoslahti, E. Cell attachment activity of fibronectin can be duplicated by small synthetic fragments of the molecule. Nature 309, 30-33 (1984); Ruoslahti (2003); Eliceiri and Cheresh (2001); Ruoslahti (2002); Arap et al. (1998); Curnis et al. (2004); Sipkins et al. (1998); Murphy et al. (2008)), and a CendR motif that mediates penetration. iRGD readily adheres to cultured cells expressing αv integrins, and is internalized far more effectively than other RGD peptides. Internalization was dependent on expression of neuropilin-1, the receptor for the CendR motif iRGD coupled to a payload of fluorescein, phage, or artificial nanoparticles, accumulated around tumor vessels in vivo, spread through the tumor interstitium, and became internalized within tumor cells in various tumor models. Systemic administration of iRGD micelles labeled with a near infrared dye produced a strong and specific tumor signal in whole body imaging of mice. The CendR element in iRGD is an activatable CendR element that is activated, likely by cleavage after the Lys/Arg, to allow the peptide to mediate internalization.

Internalizing NGR (iNGR) refers to peptides that combine a NGR motif and a CendR element. For example, NGR peptide having the sequence K/RNGR (SEQ ID NO:46) can be effective in orchestrating extravasation and spreading of linked payloads within tumor tissue, and subsequently internalizing within tumor cells. The iNGR peptide incorporates two functional elements: the NGR motif that gives tumor specificity, and a CendR motif that mediates penetration. Another example of an iNGR peptide is NGRAHA (SEQ ID NO:24). The CendR element in the iNGR peptide NGRAHA (SEQ ID NO:24) is an activatable CendR element that is activated, likely by cleavage after the Arg, to allow the peptide to mediate internalization.

Accessory molecules can be any molecule, compound, component, etc. that has a useful function and that can be used in combination with a CAR composition, CAR conjugate, CAR molecule, CAR protein, CAR peptide, and/or composition. Examples of useful accessory molecules include homing molecules, targeting molecules, affinity ligands, cell penetrating molecules, endosomal escape molecules, subcellular targeting molecules, nuclear targeting molecules. Different accessory molecules can have similar or different functions from each other.

Molecules that target, home, or have affinity for certain molecules, structures, cells, tissues, etc. are particularly useful as accessory molecules. In addition to the homing peptides described elsewhere herein, there are numerous molecules and compounds known that have affinity for particular target molecules, structures, cells, tissues, etc. and can aid in accumulating and/or directing the disclosed components and compositions to desired targets. For convenience, such affinity effects can be referred to as homing. Descriptions of homing and homing effects elsewhere herein can be applied to these molecules.

An affinity ligand is a molecule that interacts specifically with a particular molecule, moiety, cell tissue, etc. The molecule, moiety, cell tissue, etc. that interacts specifically with an affinity ligand is referred to herein as a target or target molecule, moiety, cell tissue, etc. It is to be understood that the term target molecule refers to both separate molecules and to portions of such molecules, such as an epitope of a protein, that interacts specifically with an affinity ligand. Antibodies, either member of a receptor/ligand pair, synthetic polyamides (Dervan and Burli, Sequence-specific DNA recognition by polyamides. Curr Opin Chem Biol, 3(6):688-93 (1999); Wemmer and Dervan, Targeting the minor groove of DNA. Curr Opin Struct Biol, 7(3):355-61 (1997)), and other molecules with specific binding affinities are examples of affinity ligands.

An affinity ligand that interacts specifically with a particular target molecule is said to be specific for that target molecule. For example, where the affinity ligand is an antibody that binds to a particular antigen, the affinity ligand is said to be specific for that antigen. The antigen is the target molecule. The affinity ligand can also be referred to as being specific for a particular target molecule. Examples of useful affinity ligands are antibodies, ligands, binding proteins, receptor proteins, haptens, aptamers, carbohydrates, lectins, folic acid, synthetic polyamides, and oligonucleotides. Useful binding proteins include DNA binding proteins. Useful DNA binding proteins include zinc finger motifs, leucine zipper motifs, and helix-turn-helix motifs. These motifs can be combined in the same affinity ligand.

Antibodies are useful as the affinity ligands. Antibodies can be obtained commercially or produced using well established methods. For example, Johnstone and Thorpe, Immunochemistry In Practice (Blackwell Scientific Publications, Oxford, England, 1987) on pages 30-85, describe general methods useful for producing both polyclonal and monoclonal antibodies. The entire book describes many general techniques and principles for the use of antibodies in assay systems. Numerous antibodies and other affinity ligands are known that bind to particular proteins, carbohydrates, glycoproteins, molecules, cells, tissues, etc. Such antibodies can be used in the disclosed components and compositions.

Examples of cell penetrating peptides are described in, for example, U.S. Patent Application Publication Nos. 20100061942, 20100061932, 20100048487, 20100022466, 20100016215, 20090280058, 20090186802, 20080234183, 20060014712, 20050260756, and 20030077289, which are hereby incorporated by reference in their entirety and specifically for their description of cell penetrating peptides and motifs. Examples of endosomal escape molecules are described in, for example, U.S. Patent Application Publication Nos. 20090325866, 20090317802, 20080305119, 20070292920, 20060147997, 20050038239, 20040219169, 20030148263, 20030082143, 20020132990, and 20020068272, which are hereby incorporated by reference in their entirety and specifically for their description of endosomal escape molecules and motifs. Examples of subcellular targeting molecules are described in, for example, U.S. Patent Application Publication Nos. 2009031733, 20090258926, 20090176660, 20080311136, 20070287680, 20070157328, 20070111270, 20070111251, 20060257942, 20060154340, 20060014712, 20050281805, 20050233356, 20040005309, 20030082176, and 20010021500, which are hereby incorporated by reference in their entirety and specifically for their description of subcellular targeting molecules and motifs. Examples of nuclear targeting molecules are described in, for example, U.S. Patent Application Publication Nos. 10100143454, 20100099627, 20090305329, 20090176710, 20090087899, 20070231862, 20070212332, 20060242725, 20060233807, 20060147922, 20060070133, 20060051315, 20050147993, 20050071088, 20030166601, 20030125283, 20030083261, 20030003100, 20020068272, and 20020055174, which are hereby incorporated by reference in their entirety and specifically for their description of nuclear targeting molecules and motifs.

The disclosed CAR components can be used with any therapeutic agents since they represent a general mode and platform for aiding in delivery of therapeutic agents to cells and tissues. Thus, any therapeutic agent can be used in or with the disclosed compositions. Comprehensive lists of therapeutic agents and drugs can be found in a number of places, such as the Orange Book and other lists maintained by the U.S. Food and Drug Administration (information available at websites fda.gov/Drugs/InformationOnDrugs/ucm129662.htm and fda.gov/Drugs/InformationOnDrugs/ApprovedDrugs/default.htm) and similar lists maintained by other countries, and at clinicaltrials.gov/ (for drugs and therapeutic agents undergoing clinical trials).

As used herein, the term “moiety” is used broadly to mean a physical, chemical, or biological material that generally imparts a biologically useful function to a linked composition. A moiety can be any natural or nonnatural material including, without limitation, a biological material, such as a cell, phage or other virus; an organic chemical such as a small molecule; a nanoparticle, a radionuclide; a nucleic acid molecule or oligonucleotide; a polypeptide; or a peptide. For example, moieties that affect the target, such as moieties with therapeutic effect, or that facilitate detection, visualization or imaging of the target, such as fluorescent molecule or radionuclides.

Components of the disclosed compositions can be combined, linked and/or coupled in any suitable manner. For example, moieties and other molecules can be associated covalently or non-covalently, directly or indirectly, with or without a linker moiety.

A composition can comprise a therapeutic agent. Useful therapeutic agents can be, for example, a cytotoxic agent, which, as used herein, can be any molecule that directly or indirectly promotes cell death. In some forms, a therapeutic agent can be a therapeutic polypeptide. As used herein, a therapeutic polypeptide can be any polypeptide with a biologically useful function.

The compositions disclosed herein can also be used to treat wounds or tissue injuries. Moieties and compositions useful for this purpose can include molecules belonging to several basic groups including anti-inflammatory agents which prevent inflammation, restenosis preventing drugs which prevent tissue growth, anti-thrombogenic drugs which inhibit or control formation of thrombus or thrombolytics, and bioactive agents which regulate tissue growth and enhance healing of the tissue.

Examples of active agents include but are not limited to steroids, fibronectin, anti-clotting drugs, anti-platelet function drugs, drugs which prevent smooth muscle cell growth on inner surface wall of vessel, heparin, heparin fragments, aspirin, coumadin, tissue plasminogen activator (TPA), urokinase, hirudin, streptokinase, antiproliferatives (methotrexate, cisplatin, fluorouracil, Adriamycin), antioxidants (ascorbic acid, beta carotene, vitamin E), antimetabolites, thromboxane inhibitors, non-steroidal and steroidal anti-inflammatory drugs, beta and calcium channel blockers, genetic materials including DNA and RNA fragments, complete expression genes, antibodies, lymphokines, growth factors, prostaglandins, leukotrienes, laminin, elastin, collagen, and integrins.

Useful therapeutic agents also can be antimicrobial agents and antimicrobial peptides. This can be particularly useful for targeting a wound or other infected sites. Thus, also disclosed are compositions in which a homing molecule that selectively homes to wounds, or plasma clots and interacts with fibrin-fibronectin is linked to an antimicrobial peptide, where the composition is selectively internalized and exhibits a high toxicity to the targeted area, and where the antimicrobial peptide has low mammalian cell toxicity when not linked to the homing molecule. As used herein, the term “antimicrobial peptide” means a naturally occurring or synthetic peptide having antimicrobial activity, which is the ability to kill or slow the growth of one or more microbes and which has low mammalian cell toxicity when not linked to a homing molecule. An antimicrobial peptide can, for example, kill or slow the growth of one or more strains of bacteria including a Gram-positive or Gram-negative bacteria, or a fungi or protozoa. Thus, an antimicrobial peptide can have, for example, bacteriostatic or bacteriocidal activity against, for example, one or more strains of Escherichia coli, Pseudomonas aeruginosa or Staphylococcus aureus. An antimicrobial peptide can have biological activity due to, for example, the ability to form ion channels through membrane bilayers as a consequence of self-aggregation.

Antimicrobial peptide can be highly basic and can have a linear or cyclic structure. As discussed further below, an antimicrobial peptide can have an amphipathic .alpha.-helical structure (see U.S. Pat. No. 5,789,542; Javadpour et al., J. Med. Chem. 39:3107-3113 (1996); and Blondelle and Houghten, Biochem. 31: 12688-12694 (1992)). An antimicrobial peptide also can be, for example, a β-strand/sheet-forming peptide as described in Mancheno et al., J. Peptide Res. 51:142-148 (1998).

An antimicrobial peptide can be a naturally occurring or synthetic peptide. Naturally occurring antimicrobial peptides have been isolated from biological sources such as bacteria, insects, amphibians, and mammals and are thought to represent inducible defense proteins that can protect the host organism from bacterial infection. Naturally occurring antimicrobial peptides include the gramicidins, magainins, mellitins, defensins and cecropins (see, for example, Maloy and Kari, Biopolymers 37:105-122 (1995); Alvarez-Bravo et al., Biochem. J. 302:535-538 (1994); Bessalle et al., FEBS 274:-151-155 (1990.); and Blondelle and Houghten in Bristol (Ed.), Annual Reports in Medicinal Chemistry pages 159-168 Academic Press, San Diego). An antimicrobial peptide also can be an analog of a natural peptide, especially one that retains or enhances amphipathicity.

An antimicrobial peptide incorporated into a composition can have low mammalian cell toxicity when not linked to a homing molecule. Mammalian cell toxicity readily can be assessed using routine assays. As an example, mammalian cell toxicity can be assayed by lysis of human erythrocytes in vitro as described in Javadpour et al., 1996. An antimicrobial peptide having low mammalian cell toxicity is not lytic to human erythrocytes or requires concentrations of greater than 100 μM for lytic activity, preferably concentrations greater than 200, 300, 500 or 1000 μM.

In some embodiments, disclosed are compositions in which the antimicrobial peptide portion promotes disruption of mitochondrial membranes when internalized by eukaryotic cells. In particular, such an antimicrobial peptide preferentially disrupts mitochondrial membranes as compared to eukaryotic membranes. Mitochondrial membranes, like bacterial membranes but in contrast to eukaryotic plasma membranes, have a high content of negatively charged phospholipids. An antimicrobial peptide can be assayed for activity in disrupting mitochondrial membranes using, for example, an assay for mitochondrial swelling or another assay well known in the art. _{D}(KLAKLAK)₂, (SEQ ID NO:3) for example, is an antimicrobial peptide which induces marked mitochondrial swelling at a concentration of 10 μM, significantly less than the concentration required to kill eukaryotic cells.

An antimicrobial peptide that induces significant mitochondrial swelling at, for example, 50 μM, 40 μM, 30 μM, 20 μM, 10 μM, or less, is considered a peptide that promotes disruption of mitochondrial membranes.

An antimicrobial peptide can include, for example, the sequence (KLAKLAK)₂ (SEQ ID NO:3), (KLAKKLA)₂ (SEQ ID NO:5), (KAAKKAA)₂ (SEQ ID NO:6), or (KLGKKLG)₃ (SEQ ID NO:7), and, in one embodiment, includes the sequence _{D}(KLAKLAK)₂ (SEQ ID NO:3).

Antimicrobial peptides can have random coil conformations in dilute aqueous solutions, yet high levels of helicity can be induced by helix-promoting solvents and amphipathic media such as micelles, synthetic bilayers or cell membranes. α-Helical structures are well known in the art, with an ideal α-helix characterized by having 3.6 residues per turn and a translation of 1.5 Å per residue (5.4 Å per turn; see Creighton, Proteins: Structures and Molecular Properties W.H Freeman, New York (1984)). In an amphipathic α-helical structure, polar and non-polar amino acid residues are aligned into an amphipathic helix, which is an α-helix in which the hydrophobic amino acid residues are predominantly on one face, with hydrophilic residues predominantly on the opposite face when the peptide is viewed along the helical axis.

Antimicrobial peptides of widely varying sequence have been isolated, sharing an amphipathic α-helical structure as a common feature (Saberwal et al., Biochim. Biophys. Acta 1197:109-131 (1994)). Analogs of native peptides with amino acid substitutions predicted to enhance amphipathicity and helicity typically have increased antimicrobial activity. In general, analogs with increased antimicrobial activity also have increased cytotoxicity against mammalian cells (Maloy et al., Biopolymers 37:105-122 (1995)).

As used herein in reference to an antimicrobial peptide, the term “amphipathic α-helical structure” means an α-helix with a hydrophilic face containing several polar residues at physiological pH and a hydrophobic face containing nonpolar residues. A polar residue can be, for example, a lysine or arginine residue, while a nonpolar residue can be, for example, a leucine or alanine residue. An antimicrobial peptide having an amphipathic α-helical structure generally has an equivalent number of polar and nonpolar residues within the amphipathic domain and a sufficient number of basic residues to give the peptide an overall positive charge at neutral pH (Saberwal et al., Biochim. Biophys. Acta 1197:109-131 (1994)). One skilled in the art understands that helix-promoting amino acids such as leucine and alanine can be advantageously included in an antimicrobial peptide (see, for example, Creighton, supra, 1984). Synthetic, antimicrobial peptides having an amphipathic α-helical structure are known in the art, for example, as described in U.S. Pat. No. 5,789,542 to McLaughlin and Becker.

It is understood by one skilled in the art that these and other agents are useful therapeutic agents, which can be used separately or together in the disclosed compositions and methods. Thus, it is understood that a composition can contain one or more of such therapeutic agents and that additional components can be included as part of the composition, if desired. As a non-limiting example, it can be desirable in some cases to utilize an oligopeptide spacer between the homing molecule and the therapeutic agent (Fitzpatrick and Garnett, Anticancer Drug Des. 10:1-9 (1995)).

Other useful agents include thrombolytics, aspirin, anticoagulants, painkillers and tranquilizers, beta-blockers, ace-inhibitors, nitrates, rhythm-stabilizing drugs, and diuretics. The disclosed compositions can use any of these or similar agents.

The disclosed compositions and methods can be used to diagnose and deliver targeted therapies for pulmonary diseases such as pulmonary hypertension, interstitial lung disease, acute lung injury (ALI), acute respiratory distress syndrome (ARDS), sepsis, septic shock, sarcoidosis of the lung, pulmonary manifestations of connective tissue diseases, including systemic lupus erythematosus, rheumatoid arthritis, scleroderma, and polymyositis, dermatomyositis, bronchiectasis, asbestosis, berylliosis, silicosis, Histiocytosis X, pneumotitis, smoker's lung, bronchiolitis obliterans, the prevention of lung scarring due to tuberculosis and pulmonary fibrosis, other fibrotic diseases such as myocardial infarction, endomyocardial fibrosis, mediastinal fibrosis, myelofibrosis, retroperitoneal fibrosis, progressive massive fibrosis, pneumoconiosis, nephrogenic systemic fibrosis, keloid, arthrofibrosis, adhesive capsulitis, radiation fibrosis, fibrocystic breast condition, liver cirrhosis, hepatitis, liver fibrosis, nonalcoholic fatty liver disease, nonalcoholic steatohepatitis, sarcoidosis of the lymph nodes, or other organs; inflammatory bowel disease, crohn's disease, ulcerative colitis, primary biliary cirrhosis, pancreatitis, interstitial cystitis, chronic obstructive pulmonary disease, atherosclerosis, ischemic heart disease, vasculitis, pneumoconiosis, autoimmune diseases, angiogenic diseases, wound healing, infections, trauma injuries and systemic connective tissue diseases including systemic lupus erythematosus, rheumatoid arthritis, scleroderma, polymyositis, and dermatomyositis.

For example, if the disease is pulmonary hypertension and the desired goal is targeted pulmonary arterial vasodilation, an effective dose of CAR peptide can be co-administered with a minimal dose of systemic vasodilator to achieve targeted pulmonary vasodilation and a significant decrease in pulmonary pressure with minimal systemic hypotension.

Similarly, CAR peptide can be co-administered with other medications to increase therapeutic bioavailability, boost therapeutic efficacy, and minimize side effects. CAR may be administered in a linear or cyclical form, or in any conformation deemed physiologically appropriate as a means of conveying treatment.

In addition to targeted vasodilation, we can also deliver targeted anti-coagulation. For example, in a disease like acute lung injury, which is often marked by pulmonary intra-alveolar coagulation, targeted anti-coagulation can be delivered to the affected pulmonary area by co-administering an effective dose of CAR with an anti-coagulant such as tissue factor pathway inhibitor (TFPI) or site-inactivated factor VIIa (Welty-Wolf et al., 2001) in a minimal dose to achieve targeted pulmonary anticoagulation with minimal changes in clotting ability over the areas of the body not undergoing thrombosis. Selective pulmonary anti-coagulation can also be utilized to treat other pulmonary diseases marked by pulmonary thrombosis such as pulmonary hypertension, lung transplant rejection and others.

In a disease like chronic obstructive pulmonary disease, which is often marked by shortness of breath, CAR peptide can be co-administered to boost the effective concentration and potency of drugs to relax airway smooth muscles such as long lasting β-2 agonists such as salmeterol or formoterol (Richter, et al., 2002).

Many pulmonary diseases are often marked by a decrease in glutathione (GSH), a powerful antioxidant (Morris and Bernard, 1994). CAR peptide can be co-administered with N-Acetylcysteine (NAC), a glutathione precursor, in diseases like pulmonary fibrosis, PAH, ALI, and other pulmonary disorders to boost GSH production and scavenge reactive oxidants often found in pulmonary diseases. GSH may also serve to dampen the inflammatory immune response by binding to triggering receptor expressed on myeloid cells 1 (TREM1) and diminishing monocyte/macrophage- and neutrophil-mediated inflammatory responses. Co-administration of CAR with NAC can serve to lessen the severe inflammatory immune response that often characterizes severe pulmonary and fibrotic diseases like ALI, pulmonary hypertension, autoimmune diseases and many other conditions.

The levels of antioxidants such as Superoxide Dismutase (SOD) (Rosenfeld, et al., 1996), or synthetic superoxide dismutase mimetics like EUK-8 (Gonzalez et al., 1996) can be increased through co-administration of CAR.

Treatments for pulmonary diseases like pulmonary fibrosis, PAH and ALI can also be improved by co-administering CAR with TGF-β inhibitors like decorin. Decorin, which has been previously enhanced through direct conjugation with CAR (Järvinen and Ruoslahti, 2010), can also be co-administered with CAR to achieve the benefits of targeting without direct conjugation between the CAR and decorin molecules.

In pulmonary hypertension, pulmonary fibrosis and other pulmonary diseases, the benefits of endothelin (ET-1) receptor antagonists (Kuklin et al., 2004), prostacyclin derivatives (Olschewski et al., 1999), phosphodiesterase type 5 inhibitors (Kanthapillai et al., 2004) and oncological agents such as imatinib (Ghofrani et al., 2005) (Aono et al., 2005) can be increased for patients through the co-administration of CAR.

Other pulmonary and fibrotic disease treatments such as Ketoconazole which inhibits thromboxane and leukotriene synthesis (Sinuff et al., 1999) can be improved in its efficacy while minimizing side effects through co-administration with CAR.

Newer therapeutic approaches such as small interfering RNA (siRNA), and microRNA (miRNA) therapies (Wurdinger and Costa, 2007) can also be improved and side effects minimized through the selective targeting of diseased tissue through the co-administration of CAR.

The composition can also have one or more isotopes. Such isotopes can be useful, for example, as a therapeutic agent, as a detectable agent, or both. Examples of useful isotopes include Lutetium-177 (¹⁷⁷Lu), Rhenium-188 (¹⁸⁸Re), Gallium-68 (⁶⁸Ga), Yttrium-90 (⁹⁰Y), Technetium-99m (^{99m}Tc), Holmium-166 (¹⁶⁶Ho), Iodine-131 (¹³¹I), Indium-111 (¹¹¹In), Flourine-18 (¹⁸F), Carbon-11 (¹¹C), Nitrogen-13 (¹³N), Oxygen-15 (¹⁵O), Bromine-75 (⁷⁵Br), Bromine-76 (⁷⁶Br), Iodine-124 (¹²⁴I), Thalium-201 (²⁰¹Tl), Technetium-99 (⁹⁹Tc), and Iodine-123 (¹²³I).

The composition can also comprise a detectable agent. A variety of detectable agents are useful in the disclosed methods. As used herein, the term “detectable agent” refers to any molecule which can be detected. Useful detectable agents include moieties that can be administered in vivo and subsequently detected. Detectable agents useful in the disclosed compositions and imaging methods include yet are not limited to radiolabels and fluorescent molecules. The detectable agent can be, for example, any moiety that facilitates detection, either directly or indirectly, preferably by a non-invasive and/or in vivo visualization technique. For example, a detectable agent can be detectable by any known imaging techniques, including, for example, a radiological technique. Detectable agents can include, for example, a contrast agent. The contrast agent can be, for example, Feridex. In some embodiments, for instance, the detectable agent comprises a tantalum compound. In some embodiments, the detectable agent comprises iodine, such as radioactive iodine. In some embodiments, for instance, the detectable agent comprises an organic iodo acid, such as iodo carboxylic acid, triiodophenol, iodoform, and/or tetraiodoethylene. In some embodiments, the detectable agent comprises a non-radioactive detectable agent, e.g., a non-radioactive isotope. For example, iron oxide and Gd can be used as a non-radioactive detectable agent in certain embodiments. Detectable agents can also include radioactive isotopes, enzymes, fluorophores, and quantum dots (Qdot®). For example, the detection moiety can be an enzyme, biotin, metal, or epitope tag. Other known or newly discovered detectable markers are contemplated for use with the provided compositions. In some embodiments, for instance, the detectable agent comprises a barium compound, e.g., barium sulfate.

The detectable agent can be (or the composition can include) one or more imaging agents. Examples of imaging agents include radiologic contrast agent, such as diatrizoic acid sodium salt dihydrate, iodine, and barium sulfate, a fluorescing imaging agent, such as Lissamine Rhodamine PE, a fluorescent or non-fluorescent stain or dye, for example, that can impart a visible color or that reflects a characteristic spectrum of electromagnetic radiation at visible or other wavelengths, for example, infrared or ultraviolet, such as Rhodamine, a radioisotope, a positron-emitting isotope, such as ¹⁸F or ¹²⁴I (although the short half-life of a positron-emitting isotope may impose some limitations), a metal, a ferromagnetic compound, a paramagnetic compound, such as gadolinium, a superparamagnetic compound, such as iron oxide, and a diamagnetic compound, such as barium sulfate. Imaging agents can be selected to optimize the usefulness of an image produced by a chosen imaging technology. For example, the imaging agent can be selected to enhance the contrast between a feature of interest, such as a gastrointestinal polyp, and normal gastrointestinal tissue. Imaging can be accomplished using any suitable imaging techniques such as X-Ray, computed tomography (CT), MRI, Positron Emission Tomography (PET) or SPECT. In some forms, the composition can be coupled to a nuclear medicine imaging agent such as Indium-III or Technetium-99, to PET imaging agents, or to MRI imaging agents such as nanoparticles.

Examples of imaging techniques include magnetic resonance imaging (MRI), computerized tomography (CT), single photon emission computerized tomography (SPECT), and positron emission tomography (PET). Imaging agents generally can be classified as either being diagnostic or therapeutic in their application. Because of radiation's damaging effect on tissues, it is useful to target the biodistribution of radiopharmaceuticals as accurately as possible. PET can use imaging agents labeled with, for example, the positron-emitters such as ¹⁸F, ¹¹C, ¹³N and ¹⁵O, ⁷⁵Br, ⁷⁶Br and ¹²⁴I. SPECT can use imaging agents labeled with, for example, the single-photon-emitters such as ²⁰¹Tl, ⁹⁹Tc, ¹²³I, and ¹³¹I.

Glucose-based and amino acid-based compounds can be used as imaging agents. Amino acid-based compounds are more useful in analyzing tumor cells, due to their faster uptake and incorporation into protein synthesis. Of the amino acid-based compounds, ¹¹C- and ¹⁸F-containing compounds have been used with success. ¹¹C-containing radiolabeled amino acids suitable for imaging include, for example, L[1-¹¹C]leucine (Keen et al. J. Cereb. Blood Flow Metab. 1989 (9):429-45), L[1-¹¹C]tyrosine (Wiesel et al. J. Nucl. Med. 1991 (32):2041-49), L[methyl-¹¹C]methionine (Comar et al. Eur. J. Nucl. Med. 1976 (1):11-14) and L-[1-¹¹C]methionine (Bolster et al. Appl. Radiat. Isot. 1986 (37):1069-70).

PET involves the detection of gamma rays in the form of annihilation photons from short-lived positron emitting radioactive isotopes including, but not limited to, ¹⁸F with a half-life of approximately 110 minutes, ¹¹C with a half-life of approximately 20 minutes, ¹³N with a half-life of approximately 10 minutes and ¹⁵O with a half-life of approximately 2 minutes, using the coincidence method. For PET imaging studies, compounds such as [¹¹C]meta-hydroxyephedrine (HED) and 2-[¹⁸F]fluoro-2-deoxy-D-glucose (FDG) can be used. SPECT can use longer-lived isotopes including, but not limited to, ⁹⁹mTc with a half-life of approximately 6 hours and ²⁰¹Tl with a half-life of approximately 74 hours. Radio-iodinated meta-iodobenzylguanidine (MIBG) is a radiotracing agent that can be used in nuclear medicine imaging studies.

The disclosed CAR compositions can be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. The materials can be in solution, suspension (for example, incorporated into microparticles, liposomes, or cells).

The CAR compositions can be used therapeutically in combination with a pharmaceutically acceptable carrier. Suitable carriers and their formulations are described in Remington: The Science and Practice of Pharmacy (current edition) ed. A. R. Gennaro, Mack Publishing Company, Easton, Pa. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

The preparation can be administered to a subject or organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to a subject or organism.

Herein the term “active ingredient” refers to the preparation accountable for the biological effect. For example CAR peptides, CAR compositions, CAR conjugates, CAR molecules, CAR proteins, and compositions that have a biological effect can be considered active ingredients.

As used herein, the phrases “physiologically acceptable carrier” and “pharmaceutically acceptable carrier” which can be interchangeably used refer to a carrier or a diluent that does not cause significant irritation to a subject or organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in Remington's Pharmaceutical Sciences, Mack Publishing Co., Easton, Pa., latest edition, which is incorporated herein by reference.

Any suitable route of administration can be used for the disclosed compositions. Routes of administration can, for example, include topical, enteral, local, systemic, or parenteral. For example, administration can be intratumoral, peritumoral, epicutaneous, inhalational, enema, conjunctival, eye drops, ear drops, alveolar, nasal, intranasal, vaginal, intravaginal, transvaginal, enteral, oral, intraoral, transoral, intestinal, rectal, intrarectal, transrectal, injection, infusion, intravenous, intraarterial, intramuscular, intracerebral, intraventricular, intracerebroventricular, intracardiac, subcutaneous, intraosseous, intradermal, intrathecal, intraperitoneal, intravesical, intracavernosal, intramedullar, intraocular, intracranial, transdermal, transmucosal, transnasal, inhalational, intracisternal, epidural, peridural, intravitreal, etc.

For homing to cells and tissue, particularly suitable routes of administration include parenteral, either local or systemic. For example, particularly suitable routes of administration for homing to cells and tissues include intravenous, injection, infusion, intraarterial, intramuscular, intratumoral, peritumoral, intracerebral, intraventricular, intracerebroventricular, intracardiac, subcutaneous, intraosseous, intradermal, intrathecal, intraperitoneal, intravesical, intramedullar, intraocular, intracranial, intracisternal, epidural, peridural, and intravitreal. The disclosed compositions can be used in and with any other procedure. For example, the disclosed compositions can be administered as part of HIPEC therapy. In HIPEC a heated sterile solution containing a composition of interest is continuously circulated throughout the peritoneal cavity.

Pharmaceutical compositions can be manufactured by processes well known in the art, e.g., by means of conventional mixing, dissolving, granulating, dragee-making, levigating, emulsifying, encapsulating, entrapping or lyophilizing processes.

Pharmaceutical compositions for use in the disclosed methods thus can be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients can be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

The preparations described herein can be formulated for parenteral administration, e.g., by bolus injection or continuous infusion. Formulations for injection can be presented in unit dosage form, e.g., in ampoules or in multidose containers with optionally, an added preservative. The compositions can be suspensions, solutions or emulsions in oily or aqueous vehicles, and can contain formulatory agents such as suspending, stabilizing and/or dispersing agents.

Pharmaceutical compositions for parenteral administration include aqueous solutions of the active preparation in water-soluble form. Additionally, suspensions of the active ingredients can be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions can contain substances, which increase the viscosity of the suspension, such as sodium carboxymethyl cellulose, sorbitol or dextran. Optionally, the suspension can also contain suitable stabilizers or agents which increase the solubility of the active ingredients to allow for the preparation of highly concentrated solutions.

Alternatively, the active ingredient can be in powder form for constitution with a suitable vehicle, e.g., sterile, pyrogen-free water based solution, before use.

The disclosed compositions can be provided in any suitable formulation. For example, solid, liquid, solution, gel, slow release, timed release, etc.

Pharmaceutical compositions for use in the disclosed methods include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any preparation used in the disclosed methods, the therapeutically effective amount or dose can be estimated initially from in vitro and cell culture assays. For example, a dose can be formulated in animal models to achieve a desired circulating antibody concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl et al in The Pharmacological Basis of Therapeutics, Ch. 1 p. 1. (1975)).

Dosage amount and interval can be adjusted individually to provide the effect of enhanced wound healing (minimal effective concentration, MEC). The MEC will vary for each preparation, but can be estimated from in vitro data. Dosages necessary to achieve the MEC will depend on individual characteristics and route of administration. Binding assays can be used to determine plasma concentrations.

Dosage intervals can also be determined using the MEC value. Preparations should be administered using a regimen, which maintains plasma levels, target site measurements, or other suitable measure above the MEC for 10-90% of the time, preferable between 30-90% and most preferably 50-90%.

Depending on the severity and responsiveness of the condition to be treated, dosing can be of a single or a plurality of administrations, with course of treatment lasting from several days to several weeks or until cure is effected, diminution of the disease state is achieved, or other therapeutic effect is achieved.

The amount of a composition to be administered will, of course, be dependent on the subject being treated, the severity of the affliction, the manner of administration, the judgment of the prescribing physician, etc.

Useful dosages for CAR peptides used in the disclosed methods can be, for example, 0.3, 0.5, 0.7, 1.0, 1.5, 2.0, 2.5, 2.7, 3.0, 3.3, 3.5, 3.7, 4.0, 5.0, 6.0, 7.0, 8.0, 9.0, and 10.0 mg CAR peptide per kg subject body weight. Useful dosages for CAR peptides used in the disclosed methods can be, for example, at least 0.3, 0.5, 0.7, 1.0, 1.5, 2.0, 2.5, 2.7, 3.0, 3.3, 3.5, 3.7, 4.0, 5.0, 6.0, 7.0, 8.0, 9.0, and 10.0 mg CAR peptide per kg subject body weight. Useful dosages for CAR peptides used in the disclosed methods can be, for example, 0.3, 0.5, 0.7, 1.0, 1.5, 2.0, 2.5, 2.7, 3.0, 3.3, 3.5, 3.7, 4.0, 5.0, 6.0, 7.0, 8.0, 9.0, and 10.0 mg CAR peptide per kg subject body weight or more. Useful dosages for CAR peptides used in the disclosed methods can be, for example, approximately 0.3, 0.5, 0.7, 1.0, 1.5, 2.0, 2.5, 2.7, 3.0, 3.3, 3.5, 3.7, 4.0, 5.0, 6.0, 7.0, 8.0, 9.0, and 10.0 mg CAR peptide per kg subject body weight.

The composition can be a microparticle or a nanoparticle, such as a nanosphere, nanoshell, nanoworm, heat generating nanoshell, and the like. As used herein, “nanoshell” is a nanoparticle having a discrete dielectric or semi-conducting core section surrounded by one or more conducting shell layers. U.S. Pat. No. 6,530,944 is hereby incorporated by reference herein in its entirety for its teaching of the methods of making and using metal nanoshells. Nanoshells can be formed with, for example, a core of a dielectric or inert material such as silicon, coated with a material such as a highly conductive metal which can be excited using radiation such as near infrared light (approximately 800 to 1300 nm). Upon excitation, the nanoshells emit heat. The resulting hyperthermia can kill the surrounding cell(s) or tissue. The combined diameter of the shell and core of the nanoshells ranges from the tens to the hundreds of nanometers. Near infrared light is advantageous for its ability to penetrate tissue. Other types of radiation can also be used, depending on the selection of the nanoparticle coating and targeted cells. Examples include x-rays, magnetic fields, electric fields, and ultrasound. The particles can also be used to enhance imaging, especially using infrared diffuse photon imaging methods. Targeting molecules can be antibodies or fragments thereof, ligands for specific receptors, or other proteins specifically binding to the surface of the cells to be targeted.

Fatty acids (i.e., lipids) that can be conjugated to the disclosed CAR compositions include those that allow the efficient incorporation of the peptide into liposomes. Generally, the fatty acid is a polar lipid. Thus, the fatty acid can be a phospholipid. The provided compositions can comprise either natural or synthetic phospholipid. The phospholipids can be selected from phospholipids containing saturated or unsaturated mono or disubstituted fatty acids and combinations thereof. These phospholipids can be, for example, dioleoylphosphatidylcholine, dioleoylphosphatidylserine, dioleoylphosphatidylethanolamine, dioleoylphosphatidylglycerol, dioleoylphosphatidic acid, palmitoyloleoylphosphatidylcholine, palmitoyloleoylphosphatidylserine, palmitoyloleoylphosphatidylethanolamine, palmitoyloleoylphophatidylglycerol, palmitoyloleoylphosphatidic acid, palmitelaidoyloleoylphosphatidylcholine, palmitelaidoyloleoylphosphatidylserine, palmitelaidoyloleoylphosphatidylethanolamine, palmitelaidoyloleoylphosphatidylglycerol, palmitelaidoyloleoylphosphatidic acid, myristoleoyloleoylphosphatidylcholine, myristoleoyloleoylphosphatidylserine, myristoleoyloleoylphosphatidylethanoamine, myristoleoyloleoylphosphatidylglycerol, myristoleoyloleoylphosphatidic acid, dilinoleoylphosphatidylcholine, dilinoleoylphosphatidylserine, dilinoleoylphosphatidylethanolamine, dilinoleoylphosphatidylglycerol, dilinoleoylphosphatidic acid, palmiticlinoleoylphosphatidylcholine, palmiticlinoleoylphosphatidylserine, palmiticlinoleoylphosphatidylethanolamine, palmiticlinoleoylphosphatidylglycerol, palmiticlinoleoylphosphatidic acid. These phospholipids may also be the monoacylated derivatives of phosphatidylcholine (lysophophatidylidylcholine), phosphatidylserine (lysophosphatidylserine), phosphatidylethanolamine (lysophosphatidylethanolamine), phophatidylglycerol (lysophosphatidylglycerol) and phosphatidic acid (lysophosphatidic acid). The monoacyl chain in these lysophosphatidyl derivatives may be palimtoyl, oleoyl, palmitoleoyl, linoleoyl myristoyl or myristoleoyl. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds may be varied and may have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can comprise palmitoyl 16:0.

The other molecules, elements, moieties, etc. can be covalently linked to or non-covalently associated with, for example, the disclosed CAR composition, protein, peptide, or amino acid sequence. Such molecules, elements, moieties, etc. can be linked, for example, to the amino terminal end of the disclosed protein, peptide, amino acid sequence, or CAR peptide; to an internal amino acid of the disclosed protein, peptide, amino acid sequence, or CAR peptide; to the carboxy terminal end of the disclosed protein, peptide, or amino acid sequence; to the protein, peptide, amino acid sequence on the N terminal side of the CAR peptide; via a linker to the disclosed protein, peptide, amino acid sequence, or CAR peptide; or a combination. The disclosed CAR compositions can further comprise a linker connecting such molecules, elements, moieties, etc. and disclosed CAR composition, protein, peptide, amino acid sequence, or CAR peptide. The disclosed CAR composition, protein, peptide, amino acid sequence, or CAR peptide can also be conjugated to a coating molecule such as bovine serum albumin (BSA; see Tkachenko et al., (2003) J Am Chem Soc, 125, 4700-4701) that can be used to coat nanoparticles, nanoworms, nanoshells, and the like with the protein, peptide, amino acid sequence, or CAR peptide.

Protein crosslinkers that can be used to crosslink other molecules, elements, moieties, etc. to the disclosed CAR compositions, proteins, peptides, amino acid sequences, etc. are known in the art and are defined based on utility and structure and include DSS (Disuccinimidylsuberate), DSP (Dithiobis(succinimidylpropionate)), DTSSP (3,3′-Dithiobis(sulfosuccinimidylpropionate)), SULFO BSOCOES (Bis[2-(sulfosuccinimdooxycarbonyloxy) ethyl]sulfone), BSOCOES (Bis[2-(succinimdooxycarbonyloxy)ethyl]sulfone), SULFO DST (Disulfosuccinimdyltartrate), DST (Disuccinimdyltartrate), SULFO EGS (Ethylene glycolbis(succinimidylsuccinate)), EGS (Ethylene glycolbis(sulfosuccinimidylsuccinate)), DPDPB (1,2-Di[3′-(2′-pyridyldithio) propionamido]butane), BSSS (Bis(sulfosuccinimdyl) suberate), SMPB (Succinimdyl-4-(p-maleimidophenyl) butyrate), SULFO SMPB (Sulfosuccinimdyl-4-(p-maleimidophenyl) butyrate), MBS (3-Maleimidobenzoyl-N-hydroxysuccinimide ester), SULFO MBS (3-Maleimidobenzoyl-N-hydroxysulfosuccinimide ester), SIAB (N-Succinimidyl(4-iodoacetyl)aminobenzoate), SULFO SIAB (N-Sulfosuccinimidyl(4-iodoacetyl)aminobenzoate), SMCC (Succinimidyl-4-(N-maleimidomethyl)cyclohexane-1-carboxylate), SULFO SMCC (Sulfosuccinimidyl-4-(N-maleimidomethyl)cyclohexane-1-carboxylate), NHS LC SPDP (Succinimidyl-6-[3-(2-pyridyldithio) propionamido) hexanoate), SULFO NHS LC SPDP (Sulfosuccinimidyl-6-[3-(2-pyridyldithio) propionamido) hexanoate), SPDP (N-Succinimdyl-3-(2-pyridyldithio) propionate), NHS BROMOACETATE (N-Hydroxysuccinimidylbromoacetate), NHS IODOACETATE (N-Hydroxysuccinimidyliodoacetate), MPBH (4-(N-Maleimidophenyl) butyric acid hydrazide hydrochloride), MCCH (4-(N-Maleimidomethyl)cyclohexane-1-carboxylic acid hydrazide hydrochloride), MBH (m-Maleimidobenzoic acid hydrazidehydrochloride), SULFO EMCS(N-(epsilon-Maleimidocaproyloxy) sulfosuccinimide), EMCS(N-(epsilon-Maleimidocaproyloxy) succinimide), PMPI (N-(p-Maleimidophenyl) isocyanate), KMUH (N-(kappa-Maleimidoundecanoic acid) hydrazide), LC SMCC (Succinimidyl-4-(N-maleimidomethyl)-cyclohexane-1-carboxy(6-amidocaproate)), SULFO GMBS (N-(gamma-Maleimidobutryloxy) sulfosuccinimide ester), SMPH (Succinimidyl-6-(beta-maleimidopropionamidohexanoate)), SULFO KMUS (N-(kappa-Maleimidoundecanoyloxy)sulfosuccinimide ester), GMBS (N-(gamma-Maleimidobutyrloxy) succinimide), DMP (Dimethylpimelimidate hydrochloride), DMS (Dimethylsuberimidate hydrochloride), MHBH (Wood's Reagent; Methyl-p-hydroxybenzimidate hydrochloride, 98%), DMA (Dimethyladipimidate hydrochloride).

Components of composition can also be coupled using, for example, maleimide coupling. By way of illustration, components can be coupled to lipids by coupling to, for example, 1,2-distearoyl-sn-glycero-3-phosphoethanolamine-N-[maleimide(polyethylene glycol)₂₀₀₀; DSPE-PEG₂₀₀₀-maleimide] (Avanti Polar Lipids) by making use of a free cysteine sulfhydryl group on the component. The reaction can be performed, for example, in aqueous solution at room temperature for 4 hours. This coupling chemistry can be used to couple components of compositions.

The disclosed compounds, components, and compositions can also be coupled using, for example, amino group-functionalized dextran chemistry. Particles, such as, for example, nanoparticles, nanoworms, and micelles, can be coated with amino group functionalized dextran. Attachment of PEG to aminated particles increases the circulation time, presumably by reducing the binding of plasma proteins involved in opsonization (Moghimi et al., 2001). The particles can have surface modifications, for example, for reticuloendothelial system avoidance (PEG) and homing (homing molecules), endosome escape (pH-sensitive peptide; for example, Pirello et al., 2007), a detectable agent, a therapeutic compound, or a combination. To accommodate all these functions on one particle, optimization studies can be conducted to determine what proportion of the available linking sites at the surface of the particles any one of these elements should occupy to give the best combination of targeting and payload delivery. The cell internalization and/or tissue penetration of such compositions can be mediated by the disclosed CAR peptides, amino acid sequences, proteins, molecules, conjugates, and compositions.

The CAR peptides, amino acid sequences, proteins, molecules, conjugates, and compositions themselves can be coupled to other components as disclosed herein using any known technique or the techniques described herein. A maleimide function can also be used as a coupling group. These chemistries can be used to couple CAR peptides, amino acid sequences, proteins, molecules, conjugates, and compositions to each other and to other components.

CAR peptides, amino acid sequences, and proteins can also be coupled to other components using, for example, maleimide coupling. By way of illustration, CAR peptides, amino acid sequences, and proteins can be coupled to lipids by coupling to, for example, 1,2-distearoyl-sn-glycero-3-phosphoethanolamine-N-[maleimide(polyethylene glycol)₂₀₀₀; DSPE-PEG₂₀₀₀-maleimide] (Avanti Polar Lipids) by making use of a free cysteine sulfhydryl group on the CAR peptides, amino acid sequence, or protein. The reaction can be performed, for example, in aqueous solution at room temperature for 4 hours. This coupling chemistry can be used to couple the disclosed CAR peptides, amino acid sequences, and proteins to many other components, molecules and compositions.

By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, “subject” includes, but is not limited to, animals, plants, bacteria, viruses, parasites and any other organism or entity that has nucleic acid. The subject may be a vertebrate, more specifically a mammal (e.g., a human, horse, pig, rabbit, dog, sheep, goat, non-human primate, cow, cat, guinea pig or rodent), a fish, a bird or a reptile or an amphibian. In particular, pets and livestock can be a subject. The subject can be an invertebrate, such as a worm or an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In the context of endometriosis and endometriosis cells, it is understood that a subject is a subject that has or can have endometriosis and/or endometriosis cells.

Tissue-penetrating CAR peptides can be used to augment tissue imaging and treatment with drugs. The effect of CAR peptides on imaging can be tested. For example, optical imaging with, for example, near infrared fluorphores using a Kodak IN VIVO Fx imager and Li-Cor Odyssey imager (e.g. Simberg et al., 2007; Sugahara et al., 2009), and MRI imaging can be used. For MRI imaging, the composition can comprise an MRI contrast agent such as Feridex iron oxide nanoparticles and gadolinium compounds. These compounds can be injected into wound-bearing mice, for example, with and without a wound-homing CAR peptide or a combination of peptides, followed by imaging. The results can be use to determine effectiveness of treatments and to assess different treatment protocols for using CAR peptides with therapeutics.

Combinations of different CAR peptides can be tested for optimal accumulation and distribution of the composition in the target cells and tissue by, for example, varying the dose of the drug and using the dose of the peptide that gives the maximal effect. The disclosed results show that CAR-drug combinations can reduce the amount of drug needed and therefore, the side effects, while producing the same therapeutic effect.

As defined herein, a C-terminal element (CendR element) refers to either an arginine, a lysine, or a lysine-glycine (for a type 1 CendR element), or a histidine or an amino acid sequence having the sequence X₁X₂X₃X₄, where X₁ can be R, K or H, where X₄ can be R, K, H, or KG, and where X₂ and X₃ can each be, independently, any amino acid (for a type 2 CendR element).

Type 1 CendR elements are a C-terminal arginine, a C-terminal lysine, or a C-terminal lysine-glycine pair, where glycine is at the furthest C-terminal position. In other words, in the case where a lysine is on the C terminus end, the CendR element can remain functional with a glycine on the C terminus side of the lysine. However, it is not necessary to have glycine on the end in order for the lysine residue to be functional as a C-terminal element, so that lysine can be present without glycine and still be functional. The converse is not true, however, in that glycine cannot function as a C-terminal element without the presence of lysine adjacent to it. Arginine does not require either lysine or glycine to function as a C-terminal element, as long as it remains in the furthest C-terminal position.

Type 2 CendR elements are C-terminal histidine and amino acid sequences having the sequence X₁X₂X₃X₄, where X₁ can be R, K or H, where X₄ can be R, K, H, or KG, and where X₂ and X₃ can each be, independently, any amino acid. Such CendR elements can be referred to as type 2 CendR elements. The X₂ and X₃ amino acids can be selected for specific purposes. For example, X₂, X₃, or both can be chosen to form all or a portion of a protease recognition sequence. This would be useful, for example, to specify or enable cleavage of a peptide having the CendR element as a latent or cryptic CendR element that is activated by cleavage following the X₄ amino acid. The X₁, X₂ and X₃ amino acids can also be selected, for example, to recruit additional proteins to NRP-1 molecules at the cell surface. This can be applied, for example, to modulate the selectivity and internalization and/or tissue penetration potency of CendR elements (and the compositions, conjugates, proteins, and peptides containing CendR elements). The X₂ and X₃ amino acids can also be selected to prevent protease cleavage within the X₁-X₄ motif. For example, X₂ and/or X₃ can be proline, which reduces or eliminates protease cleavage, such as by carboxypeptidase, between the proline and the next downstream amino acid. As another example, one or more of the bonds between X₁, X₂, X₃, and/or X₄ can be modified to reduce or eliminate protease cleavage at those bonds. Optionally, certain amino acids can also be excluded from use for X₂, X₃, or both. For example, if desired, G and D can be excluded from simultaneous use as X₂ and X₃, respectively. Some type 2 CendR elements can also be described as R/K/HXXR/K/H (SEQ ID NO:20), R/KXXR/K (SEQ ID NO:23), and R/K/HXXKG (SEQ ID NO:21).

For the sake of convenience, amino acid motifs that would constitute a CendR element if an arginine, lysine, lysine-glycine pair, or histidine were at the C-terminus and where the exposure in the future of the arginine, lysine, lysine-glycine pair, or histidine at the C-terminus is planned or intended, can be referred to as CendR elements or latent CendR elements.

CendR elements are described in U.S. Patent Application Publication Nos. 20090226372, 20090226372, 20090246133, and 20100322862. U.S. Patent Application Publication Nos. 20090226372, 20090226372, 20090246133, and 20100322862 are hereby incorporated herein by reference in their entirety, and specifically for their description of CendR elements.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### A. Example 1

### Enhancing Wound Healing Using CAR Peptide

It has been discovered that treatment of mice with skin wounds with CAR peptides promotes wound healing. CAR penetrates into cells and tissues in a manner that resembles the activity of these CendR peptides (Sugahara et al. 2010). Without wishing to be bound to a particular mechanism of action, it is believed that CAR enhances wound healing by improving the availability to the regenerating tissue of natural growth factors from the blood, plasma, and/or serum, and that because of the wound specificity of CAR, this effect is specific to wounds. According to this scheme, CAR allows pharmacological manipulation of the plasma->serum->plasma transition that takes place during normal tissue repair and controls tissue regeneration (FIG. 1).

In treatment experiments, intravenous administration of CAR or a control peptide was started on 24 hours after wounding. The treatment was continued for 4, 6 or 9 d in two independent treatment experiments with 15 mice in each treatment group (n=54). CAR was administered in much higher doses than the dose used in targeting CAR-decorin fusion protein to skin wounds (Jarvinen and Ruoslahti, 2010). The daily dose of 75 μg was chosen on the basis of results with CendR peptide treatments. The closure of wounds was significantly accelerated in CAR-treated mice than in controls (FIG. 2, P<0.0001 CAR vs control/mCAR for all time-points from Day 5 on).

The accelerated wound healing in the CAR-treated mice was also evident when wound closure and re-epithelization was analyzed by assessing the number of wounds that had completely closed/re-epithelialized (FIG. 3). The accelerated wound closure was due to faster re-epithelization of the wounds in the CAR-treated animals as shown in histological sections. Significantly shorter distance between the tips of the epithelial tongues was measured for the CAR-treated wounds than in controls at all time points analyzed (P<0.001, ANOVA, FIG. 4).

### B. Example 2

### Targeting of Pulmonary Arterial Hypertension with CAR Pepetides

1. Introduction

Pulmonary arterial hypertension (PAH) is a disease of the pulmonary vasculature defined by an elevated pulmonary vascular resistance, which eventually leads to right heart failure and premature death. The cause of this disease remains unknown. PAH can be idiopathic (IPAH) or associated with other conditions or exposures (secondary pulmonary hypertension), including connective tissue diseases, HIV infection, portal hypertension, and anorexigenic drug ingestion.

Most cases of severe PAH, especially IPAH, are associated with aberrant proliferation of pulmonary arterial endothelial cells and smooth muscle cells, leading to narrowing or even obliteration of the precapillary pulmonary vessel lumen (Humbert, M. et al. Cellular and molecular pathobiology of pulmonary arterial hypertension. J Am Coll Cardiol 43, 13S-24S (2004)). Thus, intimal and medial thickening of resistant arteries is typically found in the lesions. The adventitia is often markedly remodeled in patients with certain forms of collagen vascular diseases associated with severe PAH, most notably scleroderma (Cool, C. D., et al. Pulmonary hypertension: cellular and molecular mechanisms. Chest 128, 565S-571S (2005)). Inflammatory mechanisms appear to play a significant role in pathogenesis and progression of PAH. The involvement of leukocytes, such as macrophages and lymphocytes, in complex lesions of PAH has been described (Tuder, et al. Exuberant endothelial cell growth and elements of inflammation are present in plexiform lesions of pulmonary hypertension. Am J Pathol 144, 275-85 (1994); Dorfmuller, P., et al. Inflammation in pulmonary arterial hypertension. Eur Respir J 22, 358-63 (2003)).

The current therapeutic approaches for PAH consist of the administration of a variety of systemic vasodilators, which reduce pulmonary vascular resistance in some patients. However, the systemic use of vasodilators can produce adverse effects such as hypotension, impaired intrapulmonary gas exchange, and depressed cardiac function and even death (McLaughlin, V. V. et al. ACCF/AHA 2009 expert consensus document on pulmonary hypertension: a report of the American College of Cardiology Foundation Task Force on Expert Consensus Documents and the American Heart Association: developed in collaboration with the American College of Chest Physicians, American Thoracic Society, Inc., and the Pulmonary Hypertension Association. Circulation 119, 2250-94 (2009)). Drugs such as endothelin receptor antagonist may cause serious liver damage (McLaughlin, V. V. et al. ACCF/AHA 2009 expert consensus document on pulmonary hypertension: a report of the American College of Cardiology Foundation Task Force on Expert Consensus Documents and the American Heart Association: developed in collaboration with the American College of Chest Physicians, American Thoracic Society, Inc., and the Pulmonary Hypertension Association. Circulation 119, 2250-94 (2009)). These limitations of the current PAH regimens necessitate the development of target-specific interventions (Rubin, L. J. Primary pulmonary hypertension. N Engl J Med 336, 111-7 (1997)).

Delivering drugs to a diseased tissue by coupling the drug to a compound that specifically binds at the target tissue (synaphic targeting) can overcome the limitations of non-selective drug activity (Ruoslahti, E. Vascular zip codes in angiogenesis and metastasis. Biochem Soc Trans 32, 397-402 (2004); Ruoslahti, E., et al. Targeting of drugs and nanoparticles to tumors. J Cell Biol 188, 759-68 (2010)). Such a technology would provide significant therapeutic advantages such as concentrating the drug at the targeted site, which increases efficacy while decreasing side effects in other tissues. However, no compounds that would specifically bind to the blood vessels in PAH lungs are known.

In vivo screening of phage peptide libraries has been used to identify specific molecular markers in the vasculature in different organs and diseased tissues. The ‘molecular zip codes’ revealed by these screens can be used in organ-specific or lesion-specific delivery of systemically administered diagnostics and therapeutics (Ruoslahti, E. Vascular zip codes in angiogenesis and metastasis. Biochem Soc Trans 32, 397-402 (2004)). For instance, αᵥ integrins are highly expressed in tumor vasculature, where they have been accessed with peptides containing the RGD integrin recognition motif to deliver drugs, biologicals, viruses, and nanoparticles to tumor vasculature (Ruoslahti, E., et al. Targeting of drugs and nanoparticles to tumors. J Cell Biol 188, 759-68 (2010)).

Previously, a cyclic peptide, CARSKNKDC, (CAR peptide; SEQ ID NO:10) was identified via in vivo screening of phage peptide library for homing to angiogenic blood vessels during wound repair (Järvinen, T. A. & Ruoslahti, E. Molecular changes in the vasculature of injured tissues. Am J Pathol 171, 702-11 (2007)). CAR peptide accumulates in tendon and skin wounds in rats and mice with excellent selectivity, indicating that the peptide targets the vasculature of injured tissues/lesions (Järvinen, T. A. & Ruoslahti, E. Molecular changes in the vasculature of injured tissues. Am J Pathol 171, 702-11 (2007); Järvinen, T. A. H. & Ruoslahti, E. Target seeking anti-fibrotic compound enhances wound healing and suppresses scar formation. Proc Natl Acad Sci USA, (2010)). The CAR peptide specifically binds to heparin and its binding to angiogenic endothelial cells and tumor cells requires the glycosaminoglycan heparan sulfate (Järvinen, T. A. & Ruoslahti, E. Molecular changes in the vasculature of injured tissues. Am J Pathol 171, 702-11 (2007)), indicating that this peptide recognizes a specific form of heparan sulfate in the target tissues. As endothelial activation is associated with PAH (Dorfmuller, P., et al. Inflammation in pulmonary arterial hypertension. Eur Respir J 22, 358-63 (2003); Kato, G. J. et al. Levels of soluble endothelium-derived adhesion molecules in patients with sickle cell disease are associated with pulmonary hypertension, organ dysfunction, and mortality. Br J Haematol 130, 943-53 (2005)) the CAR peptide is useful for targeting vascular lesions in the diseased lung. Here, it is shown that CAR enables highly effective and selective targeting of PAH lesions.

2. Materials and Methods

i. Animals

a. Monocrotaline-Induced PAH Model.

Monocrotaline (Sigma-Aldrich, St. Louis, Mo.) was dissolved in 0.3 M hydrochloride solution and neutralized with 0.3 M sodium hydroxide solution, and adjusted pH around 7.0. Adult male Sprague-Dawley rats (150-200 g, Harlan Laboratories, Indianapolis, Ind.) were administered with a single subcutaneous injection of monocrotaline solution at a dose of 60 mg/kg body weight, while control rats were administered with 0.9% saline (Bader, M. Rat models of cardiovascular diseases. Methods Mol Biol 597, 403-14 (2010)). Rats were randomly selected and studied for peptide targeting studies on 1, 3, 7, 14 or 21 days after the treatment of monocrotaline.

b. SU5416/Hypoxia-Induced PAH Model.

Adult male Sprague-Dawley rats (approx. 200 g) were injected subcutaneously with SU5416 (20 mg/kg body weight; SUGEN Inc, South San Francisco, Calif.), which was suspended in carboxymethylcellulose (0.5% carboxymethylcellulose sodium, 0.9% sodium chloride, 0.4% polysorbate, 0.9% benzyl alcohol in deionized water). The rats were then exposed to chronic hypoxia in a hypobaric chamber (barometric pressure, 410 mm Hg: inspired O₂ tension, 76 mm Hg) for 3 weeks followed by an additional 6 weeks of reexposure to normoxia. Rats were examined at 9 weeks after the injection of SU5416 when they develop severe occlusive pulmonary arterial lesions (Abe, K. et al. Formation of plexiform lesions in experimental severe pulmonary arterial hypertension. Circulation 121, 2747-54 (2010)).

ii. Peptide Targeting Study

The following peptides were labeled with 5-carboxyfluorescein (FAM) and used for the lung targeting studies: CAR, CARSKNKDC (SEQ ID NO:10); VCAM1, CVHSPNKKCGGSK (SEQ ID NO:13); CG7C control CGGGGGGGC (SEQ ID NO:14); CAR mutant (CAR-M), CAQSNNKDC (SEQ ID NO:15). Peptides were dissolved in PBS at the concentrations of 0.5 mg/mL. Hypertensive rats were injected with peptide solution through tail vein (3.3 mg/kg). At 2 hours after the injection, rats were perfused with PBS containing 1% bovine serum albumin under the deep anesthesia and tissues were fixed by systemic perfusion with 10% buffered formalin. The organs were excised and fixed for additional 24 hours and processed for anti-fluorescein immunohistochemistry.

iii. Immunohistochemistry

To determine the localization of the peptides, paraffin-embedded tissue sections were immunostained with rabbit anti-fluoroscein isothiocyanate (FITC) antibody (Invitrogen, Carlsbad, Calif.) followed by horseradish peroxidase-labeled anti-rabbit IgG secondary antibody. The peptide localization was then visualized by DAB. Sections were counter stained with hematoxylin to visualize the region of cells. An automated staining system, Discovery XT (Ventana, Tucson, Ariz.) was used for the staining. To examine the co-localization of the peptides with macrophages or alveolar type II cells, tissue sections were doubly stained with rabbit anti-FITC antibody and mouse anti-rat-CD68 antibody; ED1 (Serotec, Raleigh, N.C.) or rabbit anti-Prosurfactant Protein C (proSP-C, Millipore, Temecula, Calif.), respectively. For FITC/proSP-C double staining, FITC staining was completed first. The tissues sections were then treated with citrate buffer (pH 6.0) at 98° C. for 10 min before proceeding to proSP-C staining Biotinylated species-specific secondary antibodies and alkaline phosphatase-labeled streptavidin (Vector Laboratories, Burlingame, Calif.) were used to visualize macrophages and alveolar type II cells with Permanent Red (Dako, Carpinteria, Calif.).

iv. Data Analysis

Images of stained slides were observed and captured with ECLIPSE 90i or 80i microscope with CCD camera DS-5M (Nikon Instruments, Melville, N.Y.). To quantify the targeting efficiency of the peptides to the lung, the immunostained sections were scanned by Aperio Scanscope XT and analyzed using ImageScope software (Aperio Technologies, Vista, Calif.). For the quantification of area and density of peptide staining, the Aperio Color Deconvolution tool was used to compare total peptide positive area and intensity of peptide staining in each animal. The thresholds were set empirically for strong, medium, and weak staining. The area of each level of staining was presented as percent of such area in the total cell area. The total stained area was normalized by hematoxylin stained area of same regions.

3. Results

i. Specific Accumulation of CAR Peptide in Monocrotaline-Induced PAH Lesions

PAH was induced in the rat by subcutaneous injection of monocrotaline (MCT), which resulted in considerable remodeling of the pulmonary vasculature 3˜4 weeks later. Fluorescein-labeled CAR peptide (FAM-CAR) or the control FAM-CG7C peptide was intravenously injected into these animals to determine peptide homing to the lung lesions. The strong green auto fluorescence of the lungs made it difficult to accurately interpret the data of direct FITC imaging. For this reason, and to preserve the histology, immunohistochemical staining with anti-FITC antibodies was used to detect the FITC-labeled peptides.

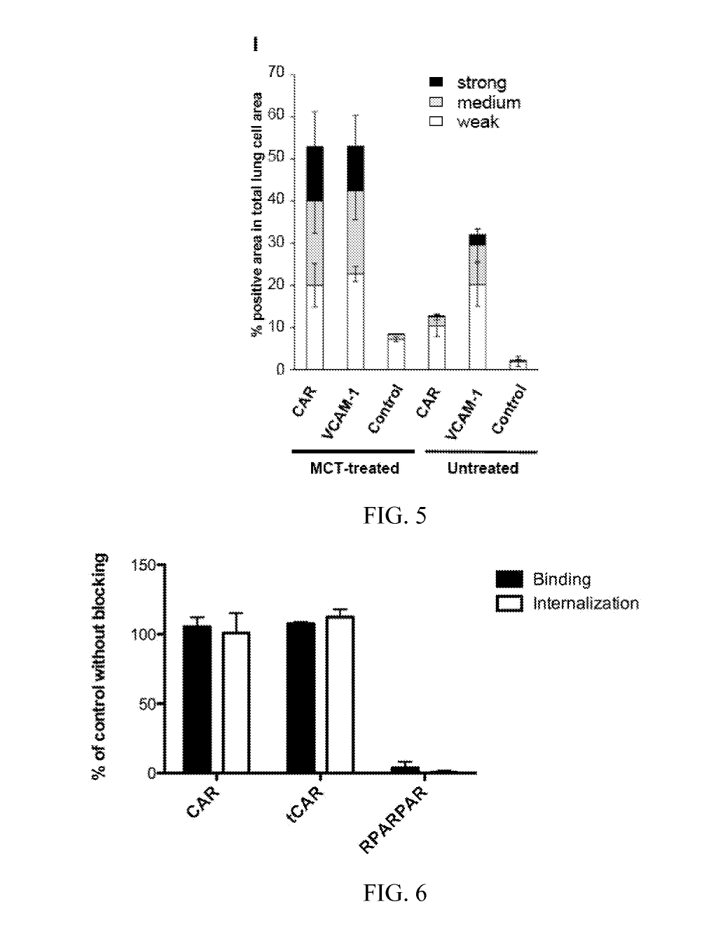

In this analysis, extensive accumulation of CAR peptide was found in the PAH lungs (FIG. 5). CAR accumulated in the endothelium and medial smooth muscle of pulmonary arteries. The accumulation of CAR was also prominent at the adventitia of pulmonary arteries. In addition, capillary vessel walls and infiltrating macrophages were strongly positive for CAR staining. Furthermore, CAR accumulated significantly in the extravascular space (interstitial space and alveolar lumen) of the PAH lungs, indicating that CAR penetrates through the vessel wall and binds to macrophages and extracellular matrix (ECM) components deposited in the injured lung; the putative receptors for CAR are heparan sulfate proteoglycans upregulated at the site of tissue injury (Järvinen, T. A. & Ruoslahti, E. Molecular changes in the vasculature of injured tissues. Am J Pathol 171, 702-11 (2007)). The staining with a Type II alveolar cell marker indicates that these cells are also positive for CAR in MCT treated hypertensive rats. In comparison, little accumulation of CAR was detected in the healthy normal lung of non-MCT-treated rats (FIG. 5). Moreover, only very weak staining was detected for the control CG7C peptide in the MCT-treated lungs (FIG. 5). These results demonstrate the specificity of CAR targeting to the PAH lung lesions.

PAH targeting activity of VCAM-1-binding peptide (VHSPNKK (amino acids 2-8 of SEQ ID NO:13)) was also examined (Kelly, K. A., et al. In Vivo Phage Display Selection Yields Atherosclerotic Plaque Targeted Peptides for Imaging. Mol Imaging Biol (2006); Kelly, K. A. et al. Detection of vascular adhesion molecule-1 expression using a novel multimodal nanoparticle. Circ Res 96, 327-36 (2005)) to compare with that of CAR. This peptide has been shown to target atherosclerotic plaques in ApoE−/− mice. VCAM-1 is highly expressed in inflammatory endothelium (Cybulsky, M. I. et al. Endothelial expression of a mononuclear leukocyte adhesion molecule during atherogenesis. Science 251, 788-91 (1991); Libby, P. Inflammation in atherosclerosis. Nature 420, 868-74 (2002)). Clinical data indicate that this is also true for the activated endothelium in PAH (Kato, G. J. et al. Levels of soluble endothelium-derived adhesion molecules in patients with sickle cell disease are associated with pulmonary hypertension, organ dysfunction, and mortality. Br J Haematol 130, 943-53 (2005)), indicating that VCAM-1 may be useful in PAH targeting. VCAM-1 binding peptide accumulated in the PAH lungs to a similar extent to CAR (FIG. 5). However, the majority of the peptide accumulation was in infiltrating macrophages and/or Type II alveolar cells, and the vasculature was less positive. Importantly, a fair amount of the VCAM-1-binding peptide also accumulated in normal healthy lungs, indicating the lack of specificity to the diseased lung, which was in contrast with the results of CAR (FIG. 5). These observations demonstrate the excellent PAH selectivity and targeting ability of the CAR peptide.

ii. Spatiotemporal Pattern of PAH Targeting by CAR

During the course of PAH lesion development, weak to moderate accumulation of CAR was detected in a few small discrete areas in the injured lung at 3 days post injury. This accumulation was absent at 7 days post injury, indicating that CAR homes to acute lung lesions. More significant and broad accumulation of CAR to the pulmonary vasculature was detected at 2 weeks post injury, and the intensity of the accumulation significantly increased in the chronic lesions in the 3rd week. The temporal pattern of CAR homing coincided with the progressive remodeling of the pulmonary vasculature and with an increase in the number of infiltrating macrophages (Bader, M. Rat models of cardiovascular diseases. Methods Mol Biol 597, 403-14 (2010)). These observations demonstrate that the lung targeting by CAR is dependent on the formation of the lung lesions. Despite the extensive homing of CAR peptide to the injured lung, accumulation in other organs was absent or very weak, except for the kidney. Intravenously administered peptides get excreted through the kidney. Thus, CAR accumulation in the kidney was observed in both MCT-treated PAH rats and untreated healthy animals. The control peptide also accumulated in the kidney. Little accumulation in healthy lungs and other organs further supports that CAR is useful for selectively targeting the PAH lesions.

iii. CAR Targets VEGFR Inhibition/Hypoxia-Induced PAH

To examine whether CAR peptide also exhibits selective targeting to PAH lesions induced by a different mechanism, another rat PAH model was employed, severe occlusive disease induced by the VEGF receptor blocker SU5416 and hypoxia (Abe, K. et al. Formation of plexiform lesions in experimental severe pulmonary arterial hypertension. Circulation 121, 2747-54 (2010)). After a single subcutaneous injection of SU5416, rats were exposed to hypoxia for 3 weeks followed by an additional 6 weeks of reexposure to normoxia. Rats were examined at 9 weeks after the injection of SU5416 when they develop severe occlusive pulmonary arterial lesions. In this model, sustained pulmonary hypertension is accompanied by the formation of complex plexiform lesions in addition to the medial wall thickening and neointima formation, thus well recapitulating the pulmonary lesion development in the pulmonary arteriopathy of human PAH (Abe, K. et al. Formation of plexiform lesions in experimental severe pulmonary arterial hypertension. Circulation 121, 2747-54 (2010)). The intravenous CAR administration resulted in prominent accumulation of the peptide throughout the PAH lungs. Notably, CAR accumulation was detected at high intensity in all layers of remodeled pulmonary arteries, i.e., endothelium, neointima, media, and adventitia. Furthermore, the CAR positive lesions included not only the endothelium and thickening medial wall but plexiform-like lesions and occluded arterioles. In contrast, only negligible signal was detected in the PAH lesion when CAR-M, an inactive CAR derivative with site-directed mutations (CAQSNNKDC (SEQ ID NO:15)), was tested, demonstrating the specificity of the CAR homing. Thus, CAR peptide exhibited a specific targeting to various forms of pulmonary arterial lesions in two distinct PAH models.

iv. Binding and Penetration of CAR into Human Cells

To assess the potential utility of CAR in targeting human PAH, binding of CAR to human endothelial cells (HUVEC) was tested in culture. When grown in culture, these endothelial cells (EC) are highly activated and mitotic with elevated expression of angiogenic genes, recapitulating the characteristics of the ECs of pathologically regenerating blood vessels (Chappey, O., et al. Endothelial cells in culture: an experimental model for the study of vascular dysfunctions. Cell Biol Toxicol 12, 199-205 (1996); Schoenfeld, J. et al. Bioinformatic analysis of primary endothelial cell gene array data illustrated by the analysis of transcriptome changes in endothelial cells exposed to VEGF-A and P1GF. Angiogenesis 7, 143-56 (2004)). CAR peptide specifically bound to the growing ECs in culture and was internalized into the cells. Reinforcing the ability of CAR to target human cells in vivo, intravenously injected CAR was shown to home to human tumor xenografts in mice with high selectivity and efficiency. Significantly, the peptide was found associated with the human tumor cells. These results indicate expression of CAR receptor in human cells, indicating that the application of CAR targeting to human PAH is feasible.

4. Discussion

In this study, highly selective PAH-targeting and tissue-penetration abilities of a recently described cyclic peptide, CAR (CARSKNKDC (SEQ ID NO:10)) were demonstrated. This is the first report on a technology that enables selective targeting of the life-threatening lung disorder, PAH. It was found that CAR homes to the pulmonary arterial endothelium and smooth muscle of the diseased lung, which are the primary tissue targets of current PAH interventions (Girgis, R. E. Emerging drugs for pulmonary hypertension. Expert Opin Emerg Drugs 15, 71-85 (2010)). It is noteworthy that CAR also accumulates in the adventitia of pulmonary arteries and in interstitial macrophages. The remodeling of the adventitia is important in the development of PAH, and macrophages recruited to the PAH lesion play a crucial role in the pathogenesis of the disease through mediating inflammatory responses. The CAR-targeting of the lung lesions may therefore offer opportunities to target multiple cell types important in the pathogenesis of PAH.

As inflammation is an element in the PAH models we used, there could be a correlation between inflammation in the lungs and CAR accumulation. However, the inflammatory response in the lungs is significantly stronger in MCT-induced PAH than in the SU5416/hypoxia model, or in human IPAH (Abe, K. et al. Formation of plexiform lesions in experimental severe pulmonary arterial hypertension. Circulation 121, 2747-54 (2010)). Nonetheless, CAR effectively targets the PAH lungs in both rat models. Moreover, CAR showed excellent specificity for the lungs in the MCT model, although this treatment is known to cause inflammation in the heart and liver as well (Campian, M. E. et al. Early inflammatory response during the development of right ventricular heart failure in a rat model. Eur J Heart Fail 12, 653-8; Copple, B. L., et al. Liver inflammation during monocrotaline hepatotoxicity. Toxicology 190, 155-69 (2003)). Therefore, inflammation alone does not appear to account for the selective CAR homing to the hypertensive lungs.

The molecular target of CAR homing in the PAH lesion is unknown. A BLAST analysis revealed a high homology of CAR sequence with the heparin-binding motif of bone morphogenetic protein-4 (BMP-4) (Järvinen, T. A. & Ruoslahti, E. Molecular changes in the vasculature of injured tissues. Am J Pathol 171, 702-11 (2007)). Whether CAR has any effect on BMP-4 signaling is unknown. In agreement with its putative heparin-binding sequence, cell binding of CAR is dependent on heparan sulfate expression of the cells (Järvinen, T. A. & Ruoslahti, E. Molecular changes in the vasculature of injured tissues. Am J Pathol 171, 702-11 (2007)), indicating that the mechanism for the vascular homing to the site of hypertensive lesions may be due to the expression of a unique glycosaminoglycan in the diseased lung. Thus, PAH vasculature appears to express a molecular zip code not present in normal lung vasculature. The identification of the receptor for CAR will be an important goal of future work that could further advance PAH targeting technology.

Currently, there are several pharmacological agents for the treatment of PAH, some of which hold promise for improved efficacy (McLaughlin, V. V. et al. ACCF/AHA 2009 expert consensus document on pulmonary hypertension: a report of the American College of Cardiology Foundation Task Force on Expert Consensus Documents and the American Heart Association: developed in collaboration with the American College of Chest Physicians, American Thoracic Society, Inc., and the Pulmonary Hypertension Association. Circulation 119, 2250-94 (2009); Girgis, R. E. Emerging drugs for pulmonary hypertension. Expert Opin Emerg Drugs 15, 71-85 (2010)). However, the lack of pulmonary vascular selectivity and associated systemic adverse effects imposes significant obstacles to successful outcomes of these therapies. When conjugated with CAR, a systemically administered fluorescence probe (FAM) was successfully delivered to and concentrated in the lung lesions. This indicates that CAR will also be useful for delivering other imaging probes and therapeutic compounds to PAH lesions. CAR accumulated significantly in the interstitial space of the PAH lungs, indicating that CAR, having specifically bound to the vasculature of the diseased lungs, extravasates and penetrates into lung parenchyma. This deep tissue penetration can make it possible to overcome the additional problem of inadequate drug penetration into the target tissues. The targeting technology described herein can be useful to develop drugs specifically engineered for targeted treatment of PAH.

### C. Example 3

### Truncated CAR Peptide Mediates Heparin Sulfate (HS)-Dependent Tumor Homing

Several CAR peptides have been used in the present studies (Table 1).

TABLE 1
Nomenclature of CAR peptides

| Peptide sequence | Name |
|---|---|
| CAR SKN KDC | CAR |
| (SEQ ID NO: 10) | |
| CAR SKN K | tCAR |
| (SEQ ID NO: 4) | |
| AR SKN K | ARSKNK |
| (SEQ ID NO: 18) | |
| KNKSRAC-CARSKNK | tCAR dimer |
| (SEQ ID NO: 4) | |

Multivalent complexes of the CendR peptide RPARPAR (SEQ ID NO:9) were used to block the neuropilin-1 binding site for peptides. RPARPAR inhibits the binding and internalization of RPARPAR phage (positive control) to cultured CHO-K cells, whereas the RPARPAR complexes have no effect on the binding and internalization of the CAR or tCAR phage (FIG. 6). These results show that the CAR and tCAR peptides do not use the CendR pathway (Teesalu et al., 2009) in their entry into cells.

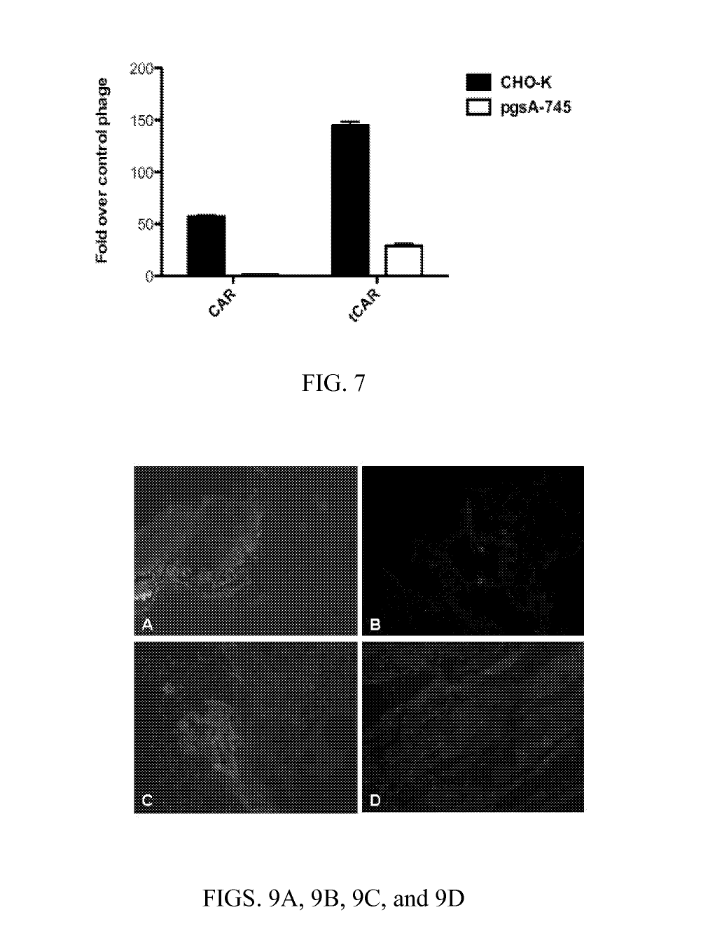

The phage internalize into CHO-K cells, whereas there is much less uptake into pgsA-745 mutant CHO-K cells, which are incapable of synthesizing glycosaminoglycans, including heparan sulfate (FIG. 7), and have previously shown not bind CAR (Jarvinen and Ruoslahti, 2008). The tCAR phage was more effectively internalized into the CHO-K cells than the CAR phage. These results show that like CAR, tCAR requires glycosaminoglycans, likely heparan sulfate, to be able to bind to cells and become internalized into them.

In vivo tumor homing of the CAR peptide was tested. Mice bearing orthotopic tumors generated with 4T1 mouse breast cells were intravenously injected with the 200 μL of 1 mM fluorescamine (FAM)-conjugated CAR peptide. The circulation time was 1 hour. The peptide accumulated in the tumor tissue and traces were also seen in the lungs. The heart muscle showed faint, uniform fluorescence. The kidneys are always positive regardless of what peptide is injected because peptides are excreted into the urine.

Homing of tCAR peptide to 4T1 tumors was examined. Confocal images of sections of the tumor and normal organs from mice injected with the 200 μL of 1 mM FAM-tCAR peptide. The circulation time was 2 hours. The tCAR peptide shows wider spreading within tumor tissue than CAR, and tCAR there was not detectable accumulation of tCAR in normal tissues.

A lack of homing of ARSKNK peptide (SEQ ID NO:18) to 4T1 tumors was determined. Confocal images of sections of the tumor and normal organs from mice injected with the 200 μL of 1 mM FAM-ARSKNK peptide. The circulation time was 1 hour. Little ARSKNK peptide accumulated in the tumor tissue, but some fluorescence was detected in the heart. These results indicate that, surprisingly, the N-terminal Cys residue is required for the tumor homing.

Homing of dimeric tCAR peptide to 4T1 tumors. Confocal images of sections of the tumor and normal organs from mice injected with the 200 μL of 1 mM FAM-KNKSRAC-CARSKNK peptide (SEQ ID NO:4). The circulation time was 1 hour. The dimeric peptide shows less tumor accumulation than the monomeric tCAR peptide. The inferior tumor homing of the dimeric tCAR shows that dimerization of tCAR through the free sulfhydryl group is not the basis of the strong tumor homing of tCAR.

In vivo tumor homing of nanoparticles coated with the tCAR peptide to 4T1 tumors was examined. Confocal images of tumors from mice injected with tCAR iron oxide nanoworms (Park et al., 2009); 5 mg/kg of iron in 130 μL were intravenously injected into the tumor mice. The circulation times were as shown. The tCAR nanoworms were initially found in and around the blood vessels, but over time extravasated and spread in the tumor tissue.

The lack of homing of tCAR nanoparticles to normal tissues in 4T1 tumor mice was determined. Confocal images of tumors from mice injected with tCAR iron oxide nanoworms (Park et al., 2009); 5 mg/kg of iron in 130 μL were intravenously injected into the tumor mice. The circulation time was 2 hours. The tCAR nanoworms accumulated in the liver and spleen because nanoparticles are cleared via the reticuloendothelial system (RES) in the liver and spleen.

A candidate receptor for the tCAR peptides was identified. Affinity chromatography of 4T1 tumor extracts on tCAR peptide immobilized onto iodoacetyl-modified agarose beads. Tumor tissue was extracted with a 200 mM glucopyranoside buffer, and the extract was incubated beads coated with tCAR peptide, followed by extensive washing with 50 mM glucopyranoside, and elution with 2 mM tCAR peptide solution. The fractions eluted from the washed affinity matrix with the tCAR peptide show the presence of a band at 98, 55, 40, and 36-kDa.

### D. Example 4

### Monocrotaline Pulmonary Hypertension Model

1. Animal Model

A rat model of monocrotaline (MCT)-induced pulmonary arterial hypertension was used for this study. Briefly, male Sprague-Dawley rats (150-200 g, Harlan Laboratories, IN) were administered with a single subcutaneous injection of monocrotaline at 60 mg/kg (Sigma-Aldrich, MO), while control rats administered 0.9% saline. Rats were randomly selected and studied for peptide distribution studies on 1, 3, 7, 14 or 21 days after the treatment of monocrotaline.

2. Peptides

The following peptides were labeled with 5-carboxyfluorescein (5FAM) and used for the lung targeting studies: CAR, 5FAM-CARSKNKDC; VCAM1, CVHSPNKKCGGSK-5FAM; Control, 5FAM-CGGGGGGGC. All peptides were synthesized by Anaspec (Anaspec Inc., CA). Peptides were resolved in PBS at the concentrations of 0.5 mg/mL.

3. Peptide Targeting Study

MCT-treated or untreated rats were injected with peptide solution at a dose of 3.3 mg/kg body weight via the tail vein. At two hours after the injection, rats were perfused with PBS containing 1% bovine serum albumin under the deep anesthesia with isofluorane at a rate of 3.0% and euthanized. Tissues were fixed by systemic perfusion with 10% buffered formalin via right ventricle. The lung was inflated by injection of 10% formalin through the trachea. Various organs were excised from the rat and fixed for additional twenty four hours and processed for immunohistochemistory.

4. Immunohistochemistry

To determine the localization of the peptides, paraffin-embedded tissue sections were immunostained with either hematoxylin and eosin or rabbit anti-fluoroscein isothiocyanate (FITC) antibody (Invitrogen, CA) followed by horseradish peroxidase-labeled anti-rabbit IgG secondary antibody. The peptide localization was then visualized by diaminobenzidine (DAB). An automated staining system, Discovery XT (Ventana, AZ) was used. To quantify the targeting efficiency of the peptides to the lung, the immunostained sections were scanned by Aperio Scanscope XT and analyzed using ImageScope software (Aperio Technologies, CA).

### E. Example 5

### Targeted Vasodilation in SU5416/Hypoxia/Normoxia-Exposed Severe Occlusive Pulmonary Hypertension

1. Animal Model

Adult male Sprague-Dawley rats weighing approximately 200 g are injected subcutaneously with SU5416 (20 mg/kg; SUGEN Inc), which is suspended in carboxymethylcellulose (0.5% [wt/vol]carboxymethylcellulose sodium, 0.9% [wt/vol] NaCl, 0.4% [vol/vol] polysorbate, 0.9% [vol/vol] benzyl alcohol in deionized water). The rats are then exposed to chronic hypoxia in a hypobaric chamber (10% O₂) for 3 weeks and are returned to normoxia (21% O₂) for an additional 2 to 10 weeks.

2. Catheterized Rats

Rats are anesthetized with intramuscular pentobarbital sodium (30 mg/kg). The rats are placed on controlled heating pads. Hemodynamic measurements are performed in anesthetized animals under normoxic conditions. Polyvinyl catheters (PV-1, internal diameter: 0.28 mm) are inserted into the right jugular vein for measurement of right ventricular systolic pressure (RVSP) and into the left jugular vein for drug administration. A microtip P-V catheter (SPR-838, Millar Instruments) is inserted into the right carotid artery and advanced into the left ventrical (LV). The signals are continuously recorded by MPVS-300 system with PowerLab/4SP, A/D converter (AD Instruments), and a personal computer. RVSP, heart rate, maximal left ventriclar systolic pressure, left ventricular end-diastolic pressure (LVEDP), mean arterial pressure (MAP), cardiac output, and stroke volume are measured. If the heart rate falls below 300 beats/min, the measurements are excluded from analysis. At the end of each hemodynamic study, the rat is sacrificed by an overdose of pentobarbital sodium, and organs are removed for various measurements and analyses.

After baseline hemodynamic measurements, a simple mixture of CAR (1 mg/300 g rat), or control peptide CARM, and fasudil (0.1, 0.3, 1, or 3 mg/kg) or each agent alone is injected intravenously, and all hemodynamic parameters are continuously monitored.

3. Immunohistochemical Staining

Organs (lung, heart, liver, spleen, and kidney) are collected after blood is flushed with 30 ml phosphate buffered saline (PBS). Lungs are inflated via trachea with 10% formalin at a constant pressure of 20 cm H₂O. After 24 hour-fixation with 10% formalin, all organs are embedded in paraffin, and sectioned at 5 mm thickness. After deparaffinization, tissue sections are pretreated with 3% hydrogen peroxidase for 10 minutes and blocked with normal horse serum for 1 hour. They are then incubated for 1 hour with an anti-fluorescein antibody (1:200; Invitrogen) as a primary antibody. After washing with PBS, the sections were incubated with biotinylated secondary antibodies, washed with PBS, and incubated in ABC Regent for 5 minutes. Diaminobenzidine was used as a substrate for the immunoperoxidase reaction. Sections were lightly counterstained with hematoxylin, and analyzed light microscopically. CAR (but not CARM) was detected in high intensity in all layers of severely remodeled arteries from lung tissue. Neither CAR nor CARM was found in other organs except for the kidney.

### F. Example 6

### Bleomycin-Induced Acute Lung Injury and Pulmonary Fibrosis Model

The bleomycin (BL) model is usually considered a model of pulmonary fibrosis, but its administration is also associated with features of acute lung injury (ALI). Bleomycin is an antineoplastic antibiotic drug isolated in 1966 from the actinomycete Streptomyces verticillus. Bleomycin forms a complex with oxygen and metals such as Fe2+, leading to the production of oxygen radicals, DNA breaks, and ultimately cell death. Bleomycin can be inactivated by bleomycin hydrolase, a cysteine protease that shows variable levels of expression in the lungs. The susceptibility of the lungs to bleomycin-induced toxicity is largely dependent on the levels of expression of bleomycin hydrolase in the lungs; species with high levels of expression, such as rabbits, are relatively resistant to bleomycin-induced toxicity, whereas species with low levels of expression, such as C57BL/6 mice, are sensitive. In addition to species-related differences in bleomycin susceptibility, there are also differences in strain susceptibility, with C57BL/6 mice being highly sensitive.

1. Animal Model

A mouse model of bleomycin induced acute lung injury and pulmonary fibrosis was used for this study. Briefly, 6 WT C57Bl/6 male mice, 8-12 weeks were weighed and anesthetized, and given bleomycin (BL) intratracheally at 4 U/kg. At 3 days (acute lung injury model) and 14 days (pulmonary fibrosis model) after BL injection, peptides were injected via the tail vein.

2. Peptides

The following peptides were labeled with 5-carboxyfluorescein (5FAM) and used for the lung targeting studies: CAR, 5FAM-CARSKNKDC; VCAM1, CVHSPNKKCGGSK-5FAM; Control, 5FAM-CGGGGGGGC. All peptides were synthesized by Anaspec (Anaspec Inc., CA). Peptides were resolved in PBS at the concentrations of 0.5 mg/mL.

3. Peptide Targeting Study

BL-treated mice were injected with peptide solution at a dose of 3.3 mg/kg body weight via the tail vein. At two hours after the injection, mice were perfused with PBS containing 1% bovine serum albumin under the deep anesthesia with isofluorane at a rate of 3.0% and euthanized. Tissues were fixed by systemic perfusion with 10% buffered formalin via right ventricle. The lung was inflated by injection of 10% formalin through the trachea. Various organs were excised from the rat and fixed for additional twenty four hours and processed for immunohistochemistory.

4. Immunohistochemistry

To determine the localization of the peptides, paraffin-embedded tissue sections were immunostained with rabbit anti-fluoroscein isothiocyanate (FITC) antibody (Invitrogen, CA) followed by horseradish peroxidase-labeled anti-rabbit IgG secondary antibody. The peptide localization was then visualized by diaminobenzidine (DAB). An automated staining system, Discovery XT (Ventana, AZ) was used. To quantify the targeting efficiency of the peptides to the lung, the immunostained sections were scanned by Aperio Scanscope XT and analyzed using ImageScope software (Aperio Technologies, CA).

5. Blood Pressure Tracing

To measure the acute effects of fasudil with and without CAR administration on the right and left ventricular systolic pressure, blood pressure measurements were performed on catheterized SU5416/hypoxia/normoxia-exposed rats with PAH (FIG. 8). Surprisingly, co-administered CAR enhanced the blood pressure lowering effect of fasudil on RVSP with only a minor reduction in LVSP, as compared to fasudil alone. Of additional importance, continuous infusion of CAR+fasudil resulted in a sustained, pulmonary-specific effect even after the cessation of the infusion. An alternative analysis was conducted, observing the same pulmonary-specific effects when comparing pressure in the RVSP to systolic aortic pressure (SAP). While the selective decrease in pulmonary pressure as measured in the RVSP is present, there is no increased CAR effect systemically when co-administered with fasudil.

### G. Example 7

### CAR Variant+Fasudil Analysis

1. Animal Model

Severe occlusive PAH rat model was used. Animals were injected with SU5416 (20 mg/kg; SUGEN Inc), followed by 3 weeks hypoxia, then followed by 2-10 weeks normoxia.

2. Peptides

The peptide administered was a 7 amino acid variant to the CAR peptide used in previous examples. This variant (tCAR; CARK) consisted of the following sequence: CARSKNK (SEQ ID NO:4). In these experiments, tCAR was administered at a dose of 3 mg/kg and fasudil administered at 1 mg/kg. CARK is an alternative designation for the truncated CAR peptide CARSKNK (SEQ ID NO:4).

3. Blood Pressure Tracing

To measure the acute effects of fasudil with and tCAR administration on the right and left ventricular systolic pressure (or systolic aortic pressure), blood pressure measurements were performed on catheterized SU5w/hypoxia/normoxia-exposed rats with PAH. Similar to CAR, tCAR co-administration enhanced the blood pressure lowering effect of fasudil on RVSP with only a minor reduction in SAP and LVSP, as compared to fasudil alone. Interestingly, administration of 10 mg/kg of fasudil 30 minutes after cessation of tCAR infusion still resulted in a sustained, pulmonary-specific effect.

Table 2 shows the results of a blood tracing experiment with fasudil co-administered with CARK. Fasudil was dosed at 1 mg/kg and CARK dosed at 3 mg/kg. Pressure measurements were observed at RVSP (mmHg) and SAP (mmHg). Table 3 shows the results of blood tracing experiments with fasudil co-administered with CARK. Fasudil was dosed at 1 mg/kg and CARK dosed at 3 mg/kg. Pressure measurements were observed at RVSP (mmHg) and LVSP (mmHg). Table 4 shows the results of a blood tracing experiment with fasudil administered after cessation of CARK infusion. Fasudil was dosed at 10 mg/kg. Pressure measurements were observed at RVSP (mmHg) and LVSP (mmHg).

TABLE 2

| | | tCAR 3 mg/kg + fasudil |
|---|---|---|
| | Baseline | 1 mg/kg |
| RVSP (mmHg) | 89.9 | 66.9 |
| SAP (mmHg) | 134.9 | 146.1 |

TABLE 3

| | | tCAR 3 mg/kg + | tCAR 3 mg/kg + |
|---|---|---|---|
| | Baseline | fasudil 1 mg/kg − 1 | fasudil 1 mg/kg − 2 |
| RVSP (mmHg) | 84.6 | 69.6 | 87.9 → 63.6 |
| LVSP (mmHg) | 140.6 | 123.9 | 136.9 → 128.9 |

TABLE 4

| | Baseline | Fasudil 10 mg/kg |
|---|---|---|
| RVSP (mmHg) | 84.6 | 27.2 |
| LVSP (mmHg) | 140.6 | 107.7 |

### H. Example 8

### CAR+Imatinib Analysis

1. Animal Model

Severe occlusive PAH rat model was used. Animals were injected with SU5416 (20 mg/kg; SUGEN Inc), followed by 3 weeks hypoxia, then followed by 2-10 weeks normoxia.

2. Peptides

The peptide administered was CAR, CARSKNKDC (SEQ ID NO:10). In this experiment, CAR was administered at a dose of 3 mg/kg and imatinib administered at 10 mg/kg.

3. Blood Pressure Tracing

To measure the acute effects of imatinib with CAR administration on the right and left ventricular systolic pressure, blood pressure measurements were performed on catheterized SU5w/hypoxia/normoxia-exposed rats with PAH. Similar to fasudil, CAR co-administration enhanced the blood pressure lowering effect of imatinib on RVSP with only a minor reduction in LVSP.

### I. Example 9

### Altered Levels of Gene Expression of Enzymes Involved in Heparan Sulfate Proteoglycan Biosynthesis Found in a Progressive Porcine Surgical Shunt Model of PAH

Heparan sulfate biosynthetic enzymes are key components in generating a myriad of distinct heparan sulfate structures that carry out multiple biologic activities. To determine whether CAR or any variants utilized the heparan sulfate pathway, an analysis was first performed to identify differential gene expression in the PAH model since CAR displayed both homing and selective therapeutic efficacy in models of PAH.

It was discovered that in the surgical shunt model of PAH, a large increase in gene expression was identified in a select group of genes, all of which are related to the heparan sulfate biosynthetic pathway. The heparan sulfate 2-O-sulfotransferase 1 (HS2ST1) gene, which encodes an enzyme responsible for catalyzing the transfer of sulfate to the C2 position of selected hexuronic acid residues within the maturing heparan sulfate, was found to be greatly increased over time in the PAH pig model.

Another gene which showed a selective increase in expression in the PAH model was exostosin 1 (EXT1), a glycosyltransferase required for the biosynthesis of heparan sulfate. Specifically, EXT1 encodes an endoplasmic reticulum-resident type II transmembrane glycosyltranferase involved in the chain elongation step of heparan sulfate biosynthesis.

Other genes identified as exhibiting an increase in expression in the PAH model were glycosyltransferase 8 domain containing 2 (GLT8D2), heparan sulfate N-deacetylase/N-sulfotransferase (NDST1) and O-linked N-acetylglucosamine transferase (OGT).

### REFERENCES

Aono Y, Nishioka Y, Inayama M, Ugai M, Kishi J, Uehara H, Izumi K, Sone S. Imatinib as a novel antifibrotic agent in bleomycin-induced pulmonary fibrosis in mice. Am J Respir Crit Care Med. 2005 Jun. 1; 171(11):1279-85.
Bishop, J R, Schuksz M, Esko, J D. Heparan sulphate proteoglycans fine-tune mammalian physiology. Nature. Vol 446(26):1030-37.
Ghofrani H A, Seeger W and Grimminger F. Imatinib for the treatment of pulmonary arterial hypertension. N Engl J Med. 2005 Sep. 29; 353(13):1412-3.
Gonzalez, P. K., Zhuang, J., Doctrow, S. R., Malfroy, B., Benson, P. F., Menconi, M. J., and Fink, M. P. (1996). Role of oxidant stress in the adult respiratory distress syndrome: evaluation of a novel antioxidant strategy in a porcine model of endotoxin-induced acute lung injury. Shock 6, Suppl 1, S23-6.
Järvinen, T. A. H., and Ruoslahti, E. (in submission). Target-seeking anti-fibrotic compound enhances tissue healing and suppresses scar formation in a wound healing model.
Järvinen, T. A. H., and Ruoslahti, E. (2007). Molecular changes in the vasculature of injured tissues. The American Journal of Pathology, 171(2), 702-711.
Kanthapillai P, Lasserson T J, Walters E H. Sildenafil for pulmonary hypertension. Cochrane Database Syst Rev 2004; 18(4):CD003562
Kuklin, V. N., Kirov, M. Y., Evgenov, O. V., Sovershaev, M. A., Sjoberg, J., Kirova, S. S., and Bjertnaes, L. J. (2004). Novel endothelin receptor antagonist attenuates endotoxin-induced lung injury in sheep. Crit Care Med., 32: 766-773.
Linhardt, R. J., Cooney, C. L., and Galliher, P. M. (1986). Polysaccharide lyases. Appl. Biochem. Biotechnol. 12:135-177.
Morris, P. E., and Bernard, G. R. (1994). Significance of glutathione in lung disease and implications for therapy. Am J Med Sci., 307(2), 119-27.
Olschewski, H., Ghofrani, H. A., Walmrath, D., Schermuly, R., Temmesfeld-Wollbruck, B., Grimminger, F., and Seeger, W. (1999). Inhaled prostacyclin and iloprost in severe pulmonary hypertension secondary to lung fibrosis. Am J Respir Crit Care Med., 160, 600-607.
Pikas D. S., Erikson, I., and Kjellen, L. (2000). Overexpression of different isoforms of glucosaminyl N-deacetylase/N-sulfotransferase results in distinct heparan sulfate N-sulfation patterns. Biochemistry, 39, 4552-4558.
Poon, G. M. K., and Garlepy, J. (2007). Cell-surface proteoglycans as molecular portals for
   cationic peptide and polymer entry into cells. Biochemical Society Transactions Vol 35, Pt 4, 788-793.
Richter, K., Janicki, S., Jorres, R. A., and Magnussen, H. (2002). Acute protection against exercise-induced bronchoconstriction by formoterol, salmeterol and terbutaline. Eur Respir J, 19, 865-871.
Rosenfeld, W. N., Davis, J. M., Parton, L., Richter, S. E., Price, A., Flaster, E., Kassem, N. (1996). Safety and pharmacokinetics of recombinant human superoxide dismutase administered intratracheally to premature neonates with respiratory distress syndrome. Pediatrics, 97(6 Pt 1), 811-7.
Sinuff T., Cook, D. J., Peterson, J. C., Fuller, H. D. (1999). Development, implementation, and evaluation of a ketoconazole practice guideline for ARDS prophylaxis. J Crit Care., 14, 1-6.
Summerford, C., and Samulski, R. J. (1997). Membrane-Associated Heparan Sulfate Proteoglycan Is a Receptor for Adeno-Associated Virus Type 2 Virions. J Virol., 72(2), 1438-1445.
Veyssier-Belot, C., and Cacoub, P. (1999). Role of endothelial and smooth muscle cells in the physiopathology and treatment management of pulmonary hypertension. Cardiovasc Res., 44, 274-282.
Welty-Wolf, K. E., Carraway, M. S., Miller, D. L., Ortel, T. L., Ezban, M., Ghio, A. J., Idell, S., and Piantadosi C. A. (2001). Coagulation blockade prevents sepsis-induced respiratory and renal failure in baboons. Am J Respir Crit Care Med., 164, 1988-1996.
Wurdinger T and Costa F F (2007). Molecular therapy in the microRNA era. The Pharmacogenomics Journal (2007) 7, 297-304.

Throughout this application, various publications are referenced. The disclosures of these publications in their entireties are hereby incorporated by reference into this application in order to more fully describe the state of the art to which this invention pertains.

It must be noted that as used herein and in the appended claims, the singular forms “a”, “an”, and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Although the description of materials, compositions, components, steps, techniques, etc. may include numerous options and alternatives, this should not be construed as, and is not an admission that, such options and alternatives are equivalent to each other or, in particular, are obvious alternatives. Thus, for example, a list of different moieties does not indicate that the listed moieties are obvious one to the other, nor is it an admission of equivalence or obviousness.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Those skilled in the art will recognize, or be able to ascertain using no more than routine experimentation, many equivalents to the specific embodiments of the method and compositions described herein. Such equivalents are intended to be encompassed by the following claims.

1. A composition comprising a wound therapeutic, wherein the only wound therapeutic in the composition is an isolated peptide, wherein peptide comprises the amino acid sequence CARSKNKDC (SEQ ID NO:10) or the amino acid sequence CARSKNK (SEQ ID NO:4).

2. The composition of claim 1, wherein the peptide is a modified peptide.

3. The composition of claim 2, wherein the peptide is a methylated peptide.

4. The composition of claim 3, wherein the methylated peptide comprises a methylated amino acid segment.

5. The composition of claim 2, wherein the peptide is N- or C-methylated in at least one position.

6. The composition of claim 1, wherein the peptide is an activatable peptide.

7. The composition of claim 6, wherein the amino acid sequence CARSKNKDC (SEQ ID NO:10) or the amino acid sequence CARSKNK (SEQ ID NO:4) is blocked by a blocking group coupled to the terminal carboxy group.

8. The composition of claim 7, wherein the blocking group comprises an amino acid or an amino acid sequence.

9. The composition of claim 1, wherein the peptide selectively homes to regenerating tissue, a site of injury, a surgical site, a site of inflammation, a site of arthritis, pulmonary arterial hypertension lung vasculature, pulmonary arterial hypertension lesions, remodeled pulmonary arteries, or interstitial space of lungs.

10. The composition of claim 1, wherein the composition selectively homes to regenerating tissue, a site of injury, a surgical site, a site of inflammation, a site of arthritis, pulmonary arterial hypertension lung vasculature, pulmonary arterial hypertension lesions, remodeled pulmonary arteries, or interstitial space of lungs.

11. The composition of claim 1, wherein the composition further comprises a carrier, vehicle, or both.

12-21. (canceled)

22. The composition of claim 1, wherein the composition binds inside blood vessels of regenerating tissue, blood vessels of wounded tissue, or lung blood vessels.

23. The composition of claim 1, wherein the composition is internalized in cells.

24. The composition of claim 1, wherein the composition penetrates tissue.

25-27. (canceled)

28. The composition of claim 1 further comprising one or more moieties.

29. The composition of claim 28, wherein the moieties are independently selected from the group consisting of a therapeutic agent, a therapeutic protein, a therapeutic compound, a therapeutic composition, a carrier, a vehicle, a virus, a phage, a viral particle, a phage particle, a viral capsid, a phage capsid, a virus-like particle, a liposome, a micelle, a bead, a nanoparticle, a microparticle, or a combination.

30. The composition of claim 1, wherein the composition further comprises one or more accessory molecules.

31. The composition of claim 1, wherein the composition has a therapeutic effect.

32. The composition of claim 31, wherein the therapeutic effect comprises a reduction in inflammation, an increase in speed of wound healing, reduction in amounts of scar tissue, decrease in pain, decrease in swelling, or decrease in necrosis.

33. (canceled)

34. A composition comprising an isolated peptide, wherein the peptide comprises the amino acid sequence CARSKNKDC (SEQ ID NO:10) or the amino acid sequence CARSKNK (SEQ ID NO:4), wherein the amount of peptide in the composition is a wound healing effective amount.

35. A composition consisting essentially of an isolated peptide, wherein the peptide comprises the amino acid sequence CARSKNKDC (SEQ ID NO:10) or the amino acid sequence CARSKNK (SEQ ID NO:4).

36. The composition of claim 1, wherein the amino acid sequence CARSKNKDC (SEQ ID NO:10) or the amino acid sequence CARSKNK (SEQ ID NO:4) is at the C-terminal end of the peptide.

37. The composition of claim 1, wherein the amino acid sequence CARSKNKDC (SEQ ID NO:10) or the amino acid sequence CARSKNK (SEQ ID NO:4) is at the N-terminal end of the peptide.

38. The composition of claim 1, wherein the amino acid sequence CARSKNKDC (SEQ ID NO:10) or the amino acid sequence CARSKNK (SEQ ID NO:4) is not at the N- or C-terminal end of the peptide.

39. A method of enhancing wound healing comprising:
exposing wounded tissue to the composition of claim 1, thereby enhancing wound healing.

40. The method of claim 39, wherein the wounded tissue is in a subject.

41. The method of claim 40, wherein the wounded tissue is exposed to the composition by administering the composition to the subject.

42. The method of claim 39, wherein the composition penetrates tissue.

43. The method of claim 39, wherein the composition penetrates lung vasculature, regenerating tissue, wounded tissue, pulmonary arterial hypertension lung vasculature, pulmonary arterial hypertension lesions, remodeled pulmonary arteries, interstitial space of lungs, a site of injury, a surgical site, ex vivo tissue, transplant tissue, ex vivo transplant tissue, a site of inflammation, or a site of arthritis.

44-45. (canceled)

46. The method of claim 39, wherein the wounded tissue is exposed to a plurality of compositions.

47. The method of claim 39, wherein the composition has a therapeutic effect.

48. The method of claim 47, wherein the therapeutic effect comprises a reduction in inflammation, an increase in speed of wound healing, reduction in amounts of scar tissue, decrease in pain, decrease in swelling, or decrease in necrosis.

49. The method of claim 47, wherein the therapeutic effect comprises pulmonary vasodilation, decrease in pulmonary pressure, anti-coagulation, airway smooth muscle relaxation, increase in glutathione (GSH), decrease in inflammatory immune response, inhibition of thromboxane synthesis, or inhibition of leukotriene synthesis.

50. The method of claim 40, wherein the subject has a disease or condition.

51. The method of claim 50, wherein the disease is pulmonary or fibrotic.

52. The method of claim 51, wherein the disease or condition is pulmonary arterial hypertension (PAH).

53. The method of claim 50, wherein the disease or condition is an autoimmune disease.

54. The method of claim 50, wherein the disease or condition is an inflammatory disease.

55. The method of claim 40, wherein the subject has one or more sites to be targeted, wherein the composition homes to one or more of the sites to be targeted.

56. The method of claim 39, wherein the peptide selectively homes to lung vasculature, regenerating tissue, wounded tissue, pulmonary arterial hypertension lung vasculature, pulmonary arterial hypertension lesions, remodeled pulmonary arteries, interstitial space of lungs, a site of injury, a surgical site, a site of inflammation, or a site of arthritis.

57. The method of claim 39, wherein the composition selectively homes to lung vasculature, regenerating tissue, wounded tissue, pulmonary arterial hypertension lung vasculature, pulmonary arterial hypertension lesions, remodeled pulmonary arteries, interstitial space of lungs, a site of injury, a surgical site, a site of inflammation, or a site of arthritis.

58. The method of claim 39, wherein the composition enhances internalization, penetration, or both of one or more blood, plasma, or serum components into or through the wounded tissue.

59. The method of claim 39, wherein the composition is not administered with any other wound therapeutic.


SEQUENCE LISTING (168 sequences)

SEQ ID NO: 1 - protein, 5 aa, Artificial Sequence
  synthetic construct; homing peptide
CGKRK

SEQ ID NO: 2 - protein, 6 aa, Artificial Sequence
  synthetic construct; homing peptide
CRKDKC

SEQ ID NO: 3 - protein, 14 aa, Artificial Sequence
  synthetic construct; membrane perturbing molecule
KLAKLAKKLAKLAK

SEQ ID NO: 4 - protein, 7 aa, Artificial Sequence
  synthetic construct; homing peptide
CARSKNK

SEQ ID NO: 5 - protein, 14 aa, Artificial Sequence
  synthetic construct; membrane perturbing molecule
KLAKKLAKLAKKLA

SEQ ID NO: 6 - protein, 14 aa, Artificial Sequence
  synthetic construct; membrane perturbing molecule
KAAKKAAKAAKKAA

SEQ ID NO: 7 - protein, 21 aa, Artificial Sequence
  synthetic construct; membrane perturbing molecule
KLGKKLGKLGKKLGKLGKKLG

SEQ ID NO: 8 - protein, 24 aa, Artificial Sequence
  synthetic construct; homing peptide
CARSKNKXXXXXXXXXXXXXXXXX

SEQ ID NO: 9 - protein, 7 aa, Artificial Sequence
  synthetic construct; homing peptide
RPARPAR

SEQ ID NO: 10 - protein, 9 aa, Artificial Sequence
  synthetic construct; skin homing peptide
CARSKNKDC

SEQ ID NO: 11 - protein, 9 aa, Artificial Sequence
  synthetic construct; cyclic homing peptide
CRGDXGPXC

SEQ ID NO: 12 - protein, 5 aa, Artificial Sequence
  synthetic construct; homing peptide
CREKA

SEQ ID NO: 13 - protein, 13 aa, Artificial Sequence
  synthetic construct; VCAM1 targeting peptide
CVHSPNKKCGGSK

SEQ ID NO: 14 - protein, 9 aa, Artificial Sequence
  synthetic construct; control peptide
CGGGGGGGC

SEQ ID NO: 15 - protein, 9 aa, Artificial Sequence
  synthetic construct; CAR mutant - homing peptide
CAQSNNKDC

SEQ ID NO: 16 - protein, 9 aa, Artificial Sequence
  synthetic construct; heart homing peptide
CRSTRANPC

SEQ ID NO: 17 - protein, 9 aa, Artificial Sequence
  synthetic construct; heart homing peptide
CPKTRRVPC

SEQ ID NO: 18 - protein, 6 aa, Artificial Sequence
  synthetic construct; homing peptide
ARSKNK

SEQ ID NO: 19 - protein, 5 aa, Artificial Sequence
  synthetic construct; heart homing peptide
RGSSS

SEQ ID NO: 20 - protein, 4 aa, Artificial Sequence
  synthetic construct; homing peptide
XXXX

SEQ ID NO: 21 - protein, 5 aa, Artificial Sequence
  synthetic construct; homing peptide
XXXKG

SEQ ID NO: 22 - protein, 5 aa, Artificial Sequence
  synthetic construct; enterokinase cleavage sequence
DDDDK

SEQ ID NO: 23 - protein, 4 aa, Artificial Sequence
  synthetic construct; homing peptide
XXXX

SEQ ID NO: 24 - protein, 6 aa, Artificial Sequence
  synthetic construct; homing peptide
NGRAHA

SEQ ID NO: 25 - protein, 7 aa, Artificial Sequence
  synthetic construct; gut homing peptide
FRVRGSP

SEQ ID NO: 26 - protein, 7 aa, Artificial Sequence
  synthetic construct; gut homing peptide
RVRGPER

SEQ ID NO: 27 - protein, 7 aa, Artificial Sequence
  synthetic construct; liver homing peptide
VKSVCRT

SEQ ID NO: 28 - protein, 7 aa, Artificial Sequence
  synthetic construct; liver homing peptide
WRQNMPL

SEQ ID NO: 29 - protein, 7 aa, Artificial Sequence
  synthetic construct; liver homing peptide
SRRFVGG

SEQ ID NO: 30 - protein, 7 aa, Artificial Sequence
  synthetic construct; liver homing peptide
ALERRSL

SEQ ID NO: 31 - protein, 7 aa, Artificial Sequence
  synthetic construct; liver homing peptide
ARRGWTL

SEQ ID NO: 32 - protein, 7 aa, Artificial Sequence
  synthetic construct; prostate homing peptide
SMSIARL

SEQ ID NO: 33 - protein, 7 aa, Artificial Sequence
  synthetic construct; prostate homing peptide
VSFLEYR

SEQ ID NO: 34 - protein, 7 aa, Artificial Sequence
  synthetic construct; prostate homing peptide
RGRWLAL

SEQ ID NO: 35 - protein, 7 aa, Artificial Sequence
  synthetic construct; ovary homing peptide
EVRSRLS

SEQ ID NO: 36 - protein, 7 aa, Artificial Sequence
  synthetic construct; ovary homing peptide
VRARLMS

SEQ ID NO: 37 - protein, 7 aa, Artificial Sequence
  synthetic construct; ovary homing peptide
RVGLVAR

SEQ ID NO: 38 - protein, 7 aa, Artificial Sequence
  synthetic construct; ovary homing peptide
RVRLVNL

SEQ ID NO: 39 - protein, 5 aa, Artificial Sequence
  synthetic construct; heart homing peptide
CRPPR

SEQ ID NO: 40 - protein, 9 aa, Artificial Sequence
  synthetic construct; heart homing peptide
CGRKSKTVC

SEQ ID NO: 41 - protein, 6 aa, Artificial Sequence
  synthetic construct; heart homing peptide
CARPAR

SEQ ID NO: 42 - protein, 6 aa, Artificial Sequence
  synthetic construct; heart homing peptide
CPKRPR

SEQ ID NO: 43 - protein, 6 aa, Artificial Sequence
  synthetic construct; heart homing peptide
CKRAVR

SEQ ID NO: 44 - protein, 9 aa, Artificial Sequence
  synthetic construct; heart homing peptide
CRNSWKPNC

SEQ ID NO: 45 - protein, 9 aa, Artificial Sequence
  synthetic construct; heart homing peptide
CSGMARTKC

SEQ ID NO: 46 - protein, 4 aa, Artificial Sequence
  synthetic construct; homing peptide
XNGR

SEQ ID NO: 47 - protein, 10 aa, Artificial Sequence
  synthetic construct; homing peptide
CQSCNGRCVR

SEQ ID NO: 48 - protein, 10 aa, Artificial Sequence
  synthetic construct; homing peptide
CRTCNGRCQV

SEQ ID NO: 49 - protein, 10 aa, Artificial Sequence
  synthetic construct; homing peptide
CVQCNGRCAL

SEQ ID NO: 50 - protein, 10 aa, Artificial Sequence
  synthetic construct; homing peptide
CRCCNGRCSP

SEQ ID NO: 51 - protein, 10 aa, Artificial Sequence
  synthetic construct; homing peptide
CASNNGRVVL

SEQ ID NO: 52 - protein, 10 aa, Artificial Sequence
  synthetic construct; homing peptide
CGRCNGRCLL

SEQ ID NO: 53 - protein, 10 aa, Artificial Sequence
  synthetic construct; homing peptide
CWLCNGRCGR

SEQ ID NO: 54 - protein, 10 aa, Artificial Sequence
  synthetic construct; homing peptide
CSKCNGRCGH

SEQ ID NO: 55 - protein, 10 aa, Artificial Sequence
  synthetic construct; homing peptide
CVWCNGRCGL

SEQ ID NO: 56 - protein, 10 aa, Artificial Sequence
  synthetic construct; homing peptide
CIRCNGRCSV

SEQ ID NO: 57 - protein, 10 aa, Artificial Sequence
  synthetic construct; homing peptide
CGECNGRCVE

SEQ ID NO: 58 - protein, 10 aa, Artificial Sequence
  synthetic construct; homing peptide
CEGVNGRRLR

SEQ ID NO: 59 - protein, 10 aa, Artificial Sequence
  synthetic construct; homing peptide
CLSCNGRCPS

SEQ ID NO: 60 - protein, 10 aa, Artificial Sequence
  synthetic construct; homing peptide
CEVCNGRCAL

SEQ ID NO: 61 - protein, 7 aa, Artificial Sequence
  synthetic construct; heart homing peptide
GGGVFWQ

SEQ ID NO: 62 - protein, 13 aa, Artificial Sequence
  synthetic construct; tumor homing peptide
CGRECPRLCQSSC

SEQ ID NO: 63 - protein, 13 aa, Artificial Sequence
  synthetic construct; homing peptide
CNGRCVSGCAGRC

SEQ ID NO: 64 - protein, 8 aa, Artificial Sequence
  synthetic construct; tumor homing peptide
CLSGSLSC

SEQ ID NO: 65 - protein, 7 aa, Artificial Sequence
  synthetic construct; tumor homing peptide
CGSLVRC

SEQ ID NO: 66 - protein, 9 aa, Artificial Sequence
  synthetic construct; homing peptide
ALNGREESP

SEQ ID NO: 67 - protein, 9 aa, Artificial Sequence
  synthetic construct; homing peptide
CVLNGRMEC

SEQ ID NO: 68 - protein, 5 aa, Artificial Sequence
  synthetic construct; homing peptide
CNGRC

SEQ ID NO: 69 - protein, 10 aa, Artificial Sequence
  synthetic construct; homing peptide
CEMCNGRCMG

SEQ ID NO: 70 - protein, 10 aa, Artificial Sequence
  synthetic construct; homing peptide
CPLCNGRCAL

SEQ ID NO: 71 - protein, 10 aa, Artificial Sequence
  synthetic construct; homing peptide
CPTCNGRCVR

SEQ ID NO: 72 - protein, 10 aa, Artificial Sequence
  synthetic construct; homing peptide
CGVCNGRCGL

SEQ ID NO: 73 - protein, 10 aa, Artificial Sequence
  synthetic construct; homing peptide
CEQCNGRCGQ

SEQ ID NO: 74 - protein, 10 aa, Artificial Sequence
  synthetic construct; homing peptide
CRNCNGRCEG

SEQ ID NO: 75 - protein, 10 aa, Artificial Sequence
  synthetic construct; homing peptide
CVLCNGRCWS

SEQ ID NO: 76 - protein, 10 aa, Artificial Sequence
  synthetic construct; homing peptide
CVTCNGRCRV

SEQ ID NO: 77 - protein, 10 aa, Artificial Sequence
  synthetic construct; homing peptide
CTECNGRCQL

SEQ ID NO: 78 - protein, 10 aa, Artificial Sequence
  synthetic construct; homing peptide
CRTCNGRCLE

SEQ ID NO: 79 - protein, 10 aa, Artificial Sequence
  synthetic construct; homing peptide
CETCNGRCVG

SEQ ID NO: 80 - protein, 10 aa, Artificial Sequence
  synthetic construct; homing peptide
CAVCNGRCGF

SEQ ID NO: 81 - protein, 10 aa, Artificial Sequence
  synthetic construct; homing peptide
CRDLNGRKVM

SEQ ID NO: 82 - protein, 10 aa, Artificial Sequence
  synthetic construct; homing peptide
CSCCNGRCGD

SEQ ID NO: 83 - protein, 10 aa, Artificial Sequence
  synthetic construct; homing peptide
CWGCNGRCRM

SEQ ID NO: 84 - protein, 10 aa, Artificial Sequence
  synthetic construct; homing peptide
CPLCNGRCAR

SEQ ID NO: 85 - protein, 10 aa, Artificial Sequence
  synthetic construct; homing peptide
CKSCNGRCLA

SEQ ID NO: 86 - protein, 10 aa, Artificial Sequence
  synthetic construct; homing peptide
CVPCNGRCHE

SEQ ID NO: 87 - protein, 8 aa, Artificial Sequence
  synthetic construct; homing peptide
CVLNGRME

SEQ ID NO: 88 - protein, 10 aa, Artificial Sequence
  synthetic construct; homing peptide
CKVCNGRCCG

SEQ ID NO: 89 - protein, 9 aa, Artificial Sequence
  synthetic construct; homing peptide
XXCNGRCXX

SEQ ID NO: 90 - protein, 11 aa, Artificial Sequence
  synthetic construct; homing peptide
CXXXNGRXXXC

SEQ ID NO: 91 - protein, 11 aa, Artificial Sequence
  synthetic construct; homing peptide
CNGRCXXXXXX

SEQ ID NO: 92 - protein, 8 aa, Artificial Sequence
  synthetic construct; lung homing peptide
VGVGEWSV

SEQ ID NO: 93 - protein, 7 aa, Artificial Sequence
  synthetic construct; intestine homing peptide
YSGKWGW

SEQ ID NO: 94 - protein, 7 aa, Artificial Sequence
  synthetic construct; uterus homing peptide
GLSGGRS

SEQ ID NO: 95 - protein, 7 aa, Artificial Sequence
  synthetic construct; adrenal gland homing peptide
LMLPRAD

SEQ ID NO: 96 - protein, 7 aa, Artificial Sequence
  synthetic construct; adrenal gland homing peptide
LPRYLLS

SEQ ID NO: 97 - protein, 9 aa, Artificial Sequence
  synthetic construct; retina homing peptide
CSCFRDVCC

SEQ ID NO: 98 - protein, 9 aa, Artificial Sequence
  synthetic construct; retina homing peptide
CRDVVSVIC

SEQ ID NO: 99 - protein, 7 aa, Artificial Sequence
  synthetic construct; gut homing peptide
YSGKWGK

SEQ ID NO: 100 - protein, 8 aa, Artificial Sequence
  synthetic construct; gut homing peptide
GISALVLS

SEQ ID NO: 101 - protein, 7 aa, Artificial Sequence
  synthetic construct; gut homing peptide
SRRQPLS

SEQ ID NO: 102 - protein, 7 aa, Artificial Sequence
  synthetic construct; gut homing peptide
MSPQLAT

SEQ ID NO: 103 - protein, 7 aa, Artificial Sequence
  synthetic construct; gut homing peptide
MRRDEQR

SEQ ID NO: 104 - protein, 7 aa, Artificial Sequence
  synthetic construct; gut homing peptide
QVRRVPE

SEQ ID NO: 105 - protein, 7 aa, Artificial Sequence
  synthetic construct; gut homing peptide
VRRGSPQ

SEQ ID NO: 106 - protein, 7 aa, Artificial Sequence
  synthetic construct, gut homing peptide
GGRGSWE

SEQ ID NO: 107 - protein, 7 aa, Artificial Sequence
  synthetic construct; heart homing peptide
HGRVRPH

SEQ ID NO: 108 - protein, 9 aa, Artificial Sequence
  synthetic construct; tumor homing peptide
CSRPRRSEC

SEQ ID NO: 109 - protein, 9 aa, Artificial Sequence
  synthetic construct; tumor homing peptide
CSRPRRSVC

SEQ ID NO: 110 - protein, 9 aa, Artificial Sequence
  synthetic construct; tumor homing peptide
CSRPRRSWC

SEQ ID NO: 111 - protein, 31 aa, Artificial Sequence
  synthetic construct; tumor homing peptide
KDEPQRRSARLSAKPAPPKPEPKPKKAPAKK

SEQ ID NO: 112 - protein, 10 aa, Artificial Sequence
  synthetic construct; tumor homing peptide
PQRRSARLSA

SEQ ID NO: 113 - protein, 10 aa, Artificial Sequence
  synthetic construct; tumor homing peptide
PKRRSARLSA

SEQ ID NO: 114 - protein, 9 aa, Artificial Sequence
  synthetic construct; brain homing peptide
CNSRLHLRC

SEQ ID NO: 115 - protein, 9 aa, Artificial Sequence
  synthetic construct; brain homing peptide
CENWWGDVC

SEQ ID NO: 116 - protein, 21 aa, Artificial Sequence
  synthetic construct; brain homing peptide
WRCVLREGPAGGCAWFNRHRL

SEQ ID NO: 117 - protein, 9 aa, Artificial Sequence
  synthetic construct; brain homing peptide
CLSSRLDAC

SEQ ID NO: 118 - protein, 8 aa, Artificial Sequence
  synthetic construct; brain homing peptide
CVLRGGRC

SEQ ID NO: 119 - protein, 9 aa, Artificial Sequence
  synthetic construct; brain homing peptide
CNSRLQLRC

SEQ ID NO: 120 - protein, 7 aa, Artificial Sequence
  synthetic construct; brain homing peptide
CGVRLGC

SEQ ID NO: 121 - protein, 8 aa, Artificial Sequence
  synthetic construct; brain homing peptide
CKDWGRIC

SEQ ID NO: 122 - protein, 8 aa, Artificial Sequence
  synthetic construct; brain homing peptide
CLDWGRIC

SEQ ID NO: 123 - protein, 8 aa, Artificial Sequence
  synthetic construct; brain homing peptide
CTRITESC

SEQ ID NO: 124 - protein, 7 aa, Artificial Sequence
  synthetic construct; brain homing peptide
CETLPAC

SEQ ID NO: 125 - protein, 8 aa, Artificial Sequence
  synthetic construct; brain homing peptide
CRTGTLFC

SEQ ID NO: 126 - protein, 8 aa, Artificial Sequence
  synthetic construct; brain homing peptide
CGRSLDAC

SEQ ID NO: 127 - protein, 9 aa, Artificial Sequence
  synthetic construct; brain homing peptide
CRHWFDVVC

SEQ ID NO: 128 - protein, 8 aa, Artificial Sequence
  synthetic construct; brain homing peptide
CANAQSHC

SEQ ID NO: 129 - protein, 8 aa, Artificial Sequence
  synthetic construct; brain homing peptide
CGNPSYRC

SEQ ID NO: 130 - protein, 20 aa, Artificial Sequence
  synthetic construct; brain homing peptide
YPCGGEAVAGVSSVRTMCSE

SEQ ID NO: 131 - protein, 20 aa, Artificial Sequence
  synthetic construct; brain homing peptide
LNCDYQGTNPATSVSVPCTV

SEQ ID NO: 132 - protein, 7 aa, Artificial Sequence
  synthetic construct; kidney homing peptide
CLPVASC

SEQ ID NO: 133 - protein, 7 aa, Artificial Sequence
  synthetic construct; kidney homing peptide
CGAREMC

SEQ ID NO: 134 - protein, 8 aa, Artificial Sequence
  synthetic construct; kidney homing peptide
CKGRSSAC

SEQ ID NO: 135 - protein, 8 aa, Artificial Sequence
  synthetic construct; kidney homing peptide
CWARAQGC

SEQ ID NO: 136 - protein, 8 aa, Artificial Sequence
  synthetic construct; kidney homing peptide
CLGRSSVC

SEQ ID NO: 137 - protein, 8 aa, Artificial Sequence
  synthetic construct; kidney homing peptide
CTSPGGSC

SEQ ID NO: 138 - protein, 8 aa, Artificial Sequence
  synthetic construct; kidney homing peptide
CMGRWRLC

SEQ ID NO: 139 - protein, 8 aa, Artificial Sequence
  synthetic construct; kidney homing peptide
CVGECGGC

SEQ ID NO: 140 - protein, 7 aa, Artificial Sequence
  synthetic construct; kidney homing peptide
CVAWLNC

SEQ ID NO: 141 - protein, 7 aa, Artificial Sequence
  synthetic construct; kidney homing peptide
CRRFQDC

SEQ ID NO: 142 - protein, 7 aa, Artificial Sequence
  synthetic construct; kidney homing peptide
CLMGVHC

SEQ ID NO: 143 - protein, 8 aa, Artificial Sequence
  synthetic construct; kidney homing peptide
CKLLSGVC

SEQ ID NO: 144 - protein, 8 aa, Artificial Sequence
  synthetic construct; kidney homing peptide
CFVGHDLC

SEQ ID NO: 145 - protein, 7 aa, Artificial Sequence
  synthetic construct; kidney homing peptide
CRCLNVC

SEQ ID NO: 146 - protein, 7 aa, Artificial Sequence
  synthetic construct; kidney homing peptide
CKLMGEC

SEQ ID NO: 147 - protein, 9 aa, Artificial Sequence
  synthetic construct; LyP-1 homing peptide
CGNKRTRGC

SEQ ID NO: 148 - protein, 7 aa, Artificial Sequence
  synthetic construct; heart homing peptide
VVLVTSS

SEQ ID NO: 149 - protein, 13 aa, Artificial Sequence
  synthetic construct; skin homing peptide
CVALCREACGEGC

SEQ ID NO: 150 - protein, 13 aa, Artificial Sequence
  synthetic construct; skin homing peptide
CSSGCSKNCLEMC

SEQ ID NO: 151 - protein, 8 aa, Artificial Sequence
  synthetic construct; skin homing peptide
CIGEVEVC

SEQ ID NO: 152 - protein, 9 aa, Artificial Sequence
  synthetic construct; skin homing peptide
CKWSRLHSC

SEQ ID NO: 153 - protein, 9 aa, Artificial Sequence
  synthetic construct; skin homing peptide
CWRGDRKIC

SEQ ID NO: 154 - protein, 9 aa, Artificial Sequence
  synthetic construct; skin homing peptide
CERVVGSSC

SEQ ID NO: 155 - protein, 9 aa, Artificial Sequence
  synthetic construct; skin homing peptide
CLAKENVVC

SEQ ID NO: 156 - protein, 13 aa, Artificial Sequence
  synthetic construct; lung homing peptide
CGFECVRQCPERC

SEQ ID NO: 157 - protein, 8 aa, Artificial Sequence
  synthetic construct; lung homing peptide
CGFELETC

SEQ ID NO: 158 - protein, 8 aa, Artificial Sequence
  synthetic construct; lung homing peptide
CTLRDRNC

SEQ ID NO: 159 - protein, 8 aa, Artificial Sequence
  synthetic construct; heart homing peptide
CLHRGNSC

SEQ ID NO: 160 - protein, 12 aa, Artificial Sequence
  synthetic construct; heart homing peptide
CRSWNKADNRSC

SEQ ID NO: 161 - protein, 8 aa, Artificial Sequence
  synthetic construct; lung homing peptide
CGKRYRNC

SEQ ID NO: 162 - protein, 8 aa, Artificial Sequence
  synthetic construct; lung homing peptide
CLRPYLNC

SEQ ID NO: 163 - protein, 12 aa, Artificial Sequence
  synthetic construct; lung homing peptide
CTVNEAYKTRMC

SEQ ID NO: 164 - protein, 12 aa, Artificial Sequence
  synthetic construct; lung homing peptide
CRLRSYGTLSLC

SEQ ID NO: 165 - protein, 12 aa, Artificial Sequence
  synthetic construct; lung homing peptide
CRPWHNQAHTEC

SEQ ID NO: 166 - protein, 9 aa, Artificial Sequence
  synthetic construct; pancreas homing peptide
SWCEPGWCR

SEQ ID NO: 167 - protein, 7 aa, Artificial Sequence
  synthetic construct; pancreas homing peptide
CKAAKNK

SEQ ID NO: 168 - protein, 7 aa, Artificial Sequence
  synthetic construct; pancreas homing peptide
CKGAKAR